(19) United States
(12) Patent Application Publication
(10) Pub. No.: US 2003/0192074 A1
(43) Pub. Date: Oct. 9, 2003

(54) Resistance gene

(76) Inventors: Paula Maria Josef Schulze-Lefert, Koln (DE); Joachim Kurth, Hurth (DE); Zhou Fasong, Norfolk (GB); Candace Elliott, Norwich Norfolk (GB); Roger Philip Wise, Ames, IA (US); Dennis Allen Halterman, Ames, IA (US); Fusheng Wei, Ames, IA (US)

Correspondence Address: Curtis Ribando, USDA ARS OTT, 1815 N University Street, Peoria, IL, 61604, US

(21) Appl. No.: 10/148,351

(22) PCT Filed: Nov. 29, 2000
(86) PCT No.: PCT/GB00/04562

(51) Int. Cl. A01H 5/00; C12Q 1/68; C07H 21/04; C12P 21/02; C12N 5/04; C07K 14/415

(52) U.S. Cl. 800/279; 435/6; 435/69.1; 435/320.1; 435/419; 530/370; 800/313; 536/23.6

(57) ABSTRACT

Disclosed are isolated nucleic acid molecules which comprise an Mla nucleotide sequence derived from an Mla locus (e.g. Mla1, 6, 12) encoding an MLA polypeptide which is capable of recognising and activating a race specific defence response in a plant into which the nucleic acid is introduced and expressed, in response to challenge with a cognate Erysiphe graminis isolate. Also disclosed are novel methods for selecting such sequences based on the determination of an Mla (AT)ₙ micro-satellite identified by the present inventors. Also provided is an novel 3 component activity assay, for assessing the ability of nucleic acid encoding a putative resistance (R) gene to confer resistance against a pathogen expressing a cognate Avr gene, which comprises the steps of: (a) selecting plant material which comprises plant cells which express a recessive gene conferring resistance against the pathogen, (b) introducing into the plant material, nucleic acid encoding (i) a detectable marker, (ii) a dominant susceptibility gene which inhibits the resistance conferred by the recessive gene, and (iii) the putative R gene, (c) challenging the plant material with the pathogen, (d) observing cells in the plant material in which the marker is expressed to determine the amount of pathogen growth present, and (e) correlating the amount of pathogen growth with the ability of the R gene to confer resistance against the pathogen. Also provided are corresponding methods and materials (e.g. vectors, polypeptides, plants, kits) based on the use of Mla nucleotide sequences or identification methods.

The present invention relates to methods and materials, particularly nucleic acids, for manipulating the resistance of plants to powdery mildew. It further relates to plants which have been modified using such methods and materials.

### PRIOR ART

Genotype specific disease resistance in plants depends on the expression of complementary avirulence (Avr) genes in the pathogen and resistance (R) genes in the host. The final outcome of a matched R-Avr interaction is incompatibility i.e. containment of the pathogen at the site of penetration.

Numerous R genes have been cloned and characterized from a wide variety of plant species. Resistance genes that function in a gene-for-gene manner generally belong to one of four general classes based on motifs that are found within the encoded protein sequence. The first three classes include a cytoplasmic protein kinase, a protein with a cytoplasmic protein kinase and extracellular leucine rich repeats (LRRs) or proteins with LRRs that appear to be located extracellularly. Members of the fourth and largest class encode cytoplasmic proteins with a nucleotide-binding site (NBS) and several LRRs. Sequence diversity within the LRRs is thought to determine recognition specificity for proteins that are otherwise quite similar. The NBS-LRR class of resistance genes can be further subdivided into proteins with a coiled-coil or Toll-interleukin-1 receptor (TIR) homology domain at the amino terminus where they may have a function in directing certain protein-protein interactions.

Both classical and molecular genetic evidence has demonstrated that resistance genes commonly belong to large, clustered families of homologous genes. Large arrays of genes with similar structures allow for recombination events that can lead to the evolution of gene products with novel recognition specificities. These recombination events may be accompanied by mutations directed at solvent-exposed residues within the LRR regions to further modify specificity.

The Mla locus in barley controls race-specific resistance to the powdery mildew pathogen, Erysiphe (=Blumeria) graminis f sp hordei.

The exceptional role of Mla is highlighted by more than 30 possibly allelic resistance specificities encoded at this locus (designated Mla1 to Mla32) each recognizing a cognate fungal Avr gene (Jahoor et al., 1995; Jorgensen, 1994; Jorgensen, 1992). Therefore, Mla can be considered a creative resistance locus (R) gene) with an enormous capacity to evolve new powdery mildew resistance specificities. Many of these powdery mildew resistance genes are believed to operate via a signalling pathway involving Rar1 and Rar2 components (see PCT/GB99/02590 of Plant Bioscience Limited). Rar1 encodes a protein containing two cysteine- and histidine-rich domains (CHORD), a motif also found in some proteins of several higher and lower eukaryotes (Shirasu et al., 1999a).

Wei et al. (1999) discloses the results of a high resolution genetic mapping and a map-based cloning protocol which aimed to delimit the Mla locus genetically and physically. Work was performed on Morex, a barley cultivar containing no known functional Mla resistance specificity, and resulted in the physical delimitation of the Mla locus to an interval of approximately 240 kb on chromosome 5S (1HS).

Within this region a combination of low pass DNA sequencing and the utilisation of degenerate PCR primers matching conserved motifs of previously isolated plant R genes enabled the identification of what was believed to be 11 resistance gene homologues (RGHS) of the NBS LRR class. The 11 RGHs were grouped into three gene families based on their sequence diversity. However since the source of this material contained no known functional Mla resistance specificity, and in view of the documented high copy number and gene sequence diversity of plant R gene loci, it could not be predicted on the basis of this publication which, if any, of the RGH DNA sequences would be related to functional Mla specificities in genetically characterised barley lines.

The characterisation and cloning of individual genes responsible for one or more functional Mla specificities is of interest because it facilitates manipulation of the pathogen resistance traits arising from those genes.

### DISCLOSURE OF THE INVENTION

The present inventors have succeeded in isolating Mla1 and Mla6. This is the first such molecular isolation of a functional resistance gene encoded at an Mla locus.

Briefly, a collection of gamma-ray and chemically-induced susceptible barley mutants (recovered following mutagenesis of cultivar Algerian containing Mla1 (C.I. 16137)-designated AlgR Mla1) were used to search for mutation-induced DNA polymorphisms by probing genomic Southern blots with DNA probes encoding RGHs which it was hoped may be at, or close to, Mla.

The activity of candidate race-specific powdery mildew R genes was assessed using a novel, 2 vector, functional assay at a single-cell level. The system potentially has a wide applicability for the detection of R genes.

Additionally, functional cDNA and genomic copies of the Mla6 allele in barley were also isolated, and the same functional assay was used to show complementation of the Mla6 phenotype in barley using an Mla6 CC-NBS-LRR gene that co-segregated with the Mla6 specificity in a high-resolution mapping population. It has also been demonstrated that Mla6 functions in wheat to confer specificity to E. graminis f. sp. hordei expressing the AvrMla6 gene. This is the first demonstration of heterologous resistance specificity in a monocotyledenous species by direct transformation.

Finally, the assay has substantiated previous genetic data that suggests that Mla6 function is dependent on Ral1.

The provision of Mla1 and Mla6 functional Mla alleles may be readily used, inter alia, to identify gene regions that may be important for recognition and signaling specificity in other Mla alleles or homologs, therefore facilitating the identification of other functional alleles.

Indeed, as shown in the Examples below, a distinctive micro-satellite (AT)ₙ repeat sequence present in Mla1 and Mla6 has been used to identify functional Mla12 gene from eight candidate cosmid clones. One cosmid clone (sp14-4) was found to contain 36 (AT) repeats. Two point mutations were found inside the gene from two susceptible mutants respectively, thereby confirming its likely identity as a functional sequence.

Thus in a first aspect of the present invention there is disclosed a nucleic acid molecule encoding a functional resistance gene encoded at an Mla locus.

Nucleic acid molecules according to the present invention may be provided isolated and/or purified from their natural environment, in substantially pure or homogeneous form, or free or substantially free of other nucleic acids of the species of origin. Where used herein, the term “isolated” encompasses all of these possibilities. The nucleic acid molecules may be wholly or partially synthetic. In particular they may be recombinant in that nucleic acid sequences which are not found together in nature (do not run contiguously) have been ligated or otherwise combined artificially.

Alternatively they may have been synthesised directly e.g. using an automated synthesiser. They may consist essentially of the gene in question.

Nucleic acid according to the present invention may include cDNA, RNA, genomic DNA and modified nucleic acids or nucleic acid analogs Where a DNA sequence is specified, e.g. with reference to a figure, unless context requires otherwise the RNA equivalent, with U substituted for T where it occurs, is encompassed. Where a nucleic acid of the invention is referred to herein, the complement of that nucleic acid will also be embraced by the invention. The ‘complement’ of a given nucleic acid (sequence) is the same length as that nucleic acid (sequence), but is 100% complementary thereto.

Where genomic nucleic acid sequences of the invention are disclosed, nucleic acids comprising any one or more (e.g. 2) introns or exons from any of those sequences are also embraced.

A resistance gene in this context is one which controls race-specific resistance to the powdery mildew pathogen, Erysiphe (=Blumeria) graminis f sp hordei i.e. a gene encoding a polypeptide capable of recognising and activating a defence response in a plant in response to challenge with an Erysiphe graminis isolate or an elicitor or Avr gene product thereof.

In the past, plant breeders have introgressed single Mla resistance specificities from barley landraces (often Hordeum vulgare subspecies spontaneum) into many cultivated barley lines, Hordeum vulgare. The Mla locus may be of any of these plants.

Nucleic acids of the first aspect may be advantageously utilised in plants which are susceptible to powdery mildew.

For example, suitable monocots include any of barley, rice, rye, wheat, maize or oat, particularly barley and wheat. Suitable dicots include Arabidopsis, tobacco, tomato, Brassicas, potato and grape vine. Other preferred plants are cucurbits, carrot, vegetable brassica, melons, capsicums, lettuce, strawberry, oilseed brassica, sugar beet, soyabeans, peas, sorghum, sunflower, tomato, pepper, chrysanthemum, carnation, poplar, eucalyptus and pine. Preferably the Mla specificity is Mla1 or Mla6. This may be tested using the methods and isolates described herein.

Thus in one embodiment of this aspect of the invention, there is disclosed a nucleic acid comprising the ‘Mla1’ nucleotide sequence of FIG. 3 or a sequence being degeneratively equivalent thereto.

A nucleic acid of the present invention may encode the ‘MLA1’ amino acid sequence of FIG. 5. Another embodiment is a nucleic acid comprising the ‘Mla6 ORF’ of Annex I or a sequence being degeneratively equivalent thereto. Further embodiments include the Mla6 cDNA (Annex II) or Mla6 gDNA (Annex III). A nucleic acid of the present invention may encode the ‘MLA6’ amino acid sequence of FIG. 10. Another embodiment is a nucleic acid comprising the ‘Mla12 cDNA’ of Annex IV or a sequence being degeneratively equivalent thereto. Further embodiments include the Mla12 genomic DNA (FIG. 11). A nucleic acid of the present invention may encode the ‘MLA12’ amino acid sequence of Annex V.

MLA6 and MLA1 are 92.2% similar (91.2% identical) at the amino acid level. The MLA12 cosmid sequence, and its corresponding cDNA clone, encode a full-length NB-LRR protein, which is 90.5% identical to Mla1, 92% to Mla6.

In a further aspect of the present invention there are disclosed nucleic acids which are variants of the sequences of the first aspect.

A variant nucleic acid molecule shares homology with, or is identical to, all or part of the coding sequence discussed above. Generally, variants may encode, or be used to isolate or amplify nucleic acids which encode, polypeptides which are capable of mediating a response against a pathogen, particularly Erysiphe graminis, and/or which will specifically bind to an antibody raised against the MLA6, MLA1 or MLA12 polypeptides of FIG. 10 or Annex V respectively.

Variants of the present invention can be artificial nucleic acids (i.e. containing sequences which have not originated naturally) which can be prepared by the skilled person in the light of the present disclosure. Alternatively they may be novel, naturally occurring, nucleic acids, which may be isolatable using the sequences of the present invention.

Thus a variant may be a distinctive part or fragment (however produced) corresponding to a portion of the sequence provided. The fragments may encode particular functional parts of the polypeptide, e.g. P-loop, middle, or LRR regions, or termini. Equally the fragments may have utility in probing for, or amplifying, the sequence provided or closely related ones. Suitable lengths of fragment, and conditions, for such processes are discussed in more detail below.

Also included are nucleic acids which have been extended at the 3′ or 5′ terminus.

Sequence variants which occur naturally may include alleles or other homologues (which may include polymorphisms or mutations at one or more bases). An example of such a homologue is shown in FIG. 4 (nucleotide sequence) and FIG. 6 (amino acid sequence). This shares 82% DNA sequence identity, and 78% amino acid sequence identity, with Mla1\MLA1 described above.

Artificial variants (derivatives) may be prepared by those skilled in the art, for instance by site directed or random mutagenesis, or by direct synthesis. Preferably the variant nucleic acid is generated either directly or indirectly (e.g. via one or amplification or replication steps) from an original nucleic acid having all or part of the sequences of the first aspect. Preferably it encodes an Erysiphe graminis resistance gene.

The term ‘variant’ nucleic acid as used herein encompasses all of these possibilities. When used in the context of polypeptides or proteins it indicates the encoded expression product of the variant nucleic acid.

Some of the aspects of the present invention relating to variants will now be discussed in more detail.

Homology (i.e. similarity or identity) may be as defined using sequence comparisons are made using BestFit and CAP programs of GCG, Wisconsin Package 10.0 from the Genetics Computer Group, Madison, Wis. Parameters are preferably set, using the default settings, as follows: Gap Creation pen: 9; Gapext pen: 2. Homology may be at the nucleotide sequence and/or encoded amino acid sequence level. Preferably, the nucleic acid and/or amino acid sequence shares at least about 50%, or 60%, or 70%, or 80% homology, most preferably at least about 90%, 95%, 96%, 97%, 98% or 99% homology with Mla1 or Mla6 or Mla12.

Thus a variant polypeptide in accordance with the present invention may include within the Mla1, Mla6 or Mla12 sequence shown in FIG. 10 or Annex V, a single amino acid or 2, 3, 4, 5, 6, 7, 8, or 9 changes, about 10, 15, 20, 30, 40 or 50 changes, or greater than about 50, 60, 70, 80, 90, 100, 200, 300 or 400 changes. In addition to one or more changes within the amino acid sequence shown, a variant polypeptide may include additional amino acids at the C-terminus and/or N-terminus. Naturally, regarding nucleic acid variants, changes to the nucleic acid which make no difference to the encoded polypeptide (i.e. ‘degeneratively equivalent’) are included within the scope of the present invention.

Thus in a further aspect of the invention there is disclosed a method of producing a derivative nucleic acid comprising the step of modifying the coding sequence of an Mla1 or Mla6 nucleic acid of the present invention (see e.g. FIGS. 3, 4, 9 or 11).

Changes to a sequence, to produce a derivative, may be by one or more of addition, insertion, deletion or substitution of one or more nucleotides in the nucleic acid, leading to the addition, insertion, deletion or substitution of one or more amino acids in the encoded polypeptide. Changes may be desirable for a number of reasons, including introducing or removing the following features: restriction endonuclease sequences; codon usage; other sites which are required for post translation modification; cleavage sites in the encoded polypeptide; motifs in the encoded polypeptide (e.g. binding sites). Leader or other targeting sequences (e.g. hydrophobic anchoring regions, potential myristoylation sites) may be added or removed from the expressed protein to determine its location following expression. All of these may assist in efficiently cloning and expressing an active polypeptide in recombinant form (as described below).

Other desirable mutation may be random or site directed mutagenesis in order to alter the activity (e.g. specificity) or stability of the encoded polypeptide. Changes may be by way of conservative variation, i.e. substitution of one hydrophobic residue such as isoleucine, valine, leucine or methionine for another, or the substitution of one polar residue for another, such as arginine for lysine, glutamic for aspartic acid, or glutamine for asparagine. As is well known to those skilled in the art, altering the primary structure of a polypeptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptides conformation. Also included are variants having non-conservative substitutions. As is well known to those skilled in the art, substitutions to regions of a peptide which are not critical in determining its conformation may not greatly affect its activity because they do not greatly alter the peptide's three dimensional structure.

In regions which are critical in determining the peptides conformation or activity such changes may confer advantageous properties on the polypeptide. Indeed, changes such as those described above may confer slightly advantageous properties on the peptide e.g. altered stability or specificity. For instance, the manipulation of LRR regions of the polypeptides encoded by the nucleic acids of the present invention may allow the production of novel resistance specificities e.g. with respect to existing or novel powdery mildew isolates.

Other methods for generating novel specificities may include mixing or incorporating sequences from related resistance genes into the Mla sequences disclosed herein. An alternative strategy for modifying Mla sequences would employ PCR as described below (Ho et al., 1989, Gene 77, 51-59) or DNA shuffling (Crameri et al., 1998, Nature 391).

In a further aspect of the present invention there is provided a method of identifying and/or cloning a nucleic acid variant from a plant which method employs a distinctive Mla1 nucleotide sequence (e.g. as present in an Mla1, Mla6 or Mla12 nucleic acid of the present invention—see e.g. FIGS. 3, 4, 9 or 12— or the complement thereof, or degenerate primers based thereon).

An oligonucleotide for use in probing or amplification reactions comprise or consist of about 30 or fewer nucleotides in length (e.g. 18, 21 or 24). Generally specific primers are upwards of 14 nucleotides in length. For optimum specificity and cost effectiveness, primers of 16-24 nucleotides in length may be preferred. Those skilled in the art are well versed in the design of primers for use processes such as PCR. If required, probing can be done with entire restriction fragments of the gene disclosed herein which may be 100's or even 1000's of nucleotides in length. Preferably the probe/primer is distinctive in the sense that it is present in all or some of the Mla sequences disclosed herein, but not in resistance gene sequences of the prior art.

For instance, the functional allele data presented herein (see e.g. FIG. 9 or FIG. 11) permits the identification of functional Mla alleles as follows.

In one embodiment, nucleotide sequence information provided herein may be used in a data-base (e.g. of expressed sequence tags, or sequence tagged sites) search to find homologous sequences, such as those which may become available in due course, and expression products of which can be tested for activity as described below. In a further embodiment, a variant in accordance with the present invention is also obtainable by means of a method which includes:

(a) providing a preparation of nucleic acid, e.g. from plant cells,

(b) providing a nucleic acid molecule which is a probe as described above,

(c) contacting nucleic acid in said preparation with said nucleic acid molecule under conditions for hybridisation of said nucleic acid molecule to any said gene or homologue in said preparation, and identifying said gene or homologue if present by its hybridisation with said nucleic acid molecule.

Probing may employ the standard Southern blotting technique. For instance DNA may be extracted from cells and digested with different restriction enzymes. Restriction fragments may then be separated by electrophoresis on an agarose gel, before denaturation and transfer to a nitrocellulose filter or nylon. Labelled probe may be hybridised to the DNA fragments on the filter and binding determined. DNA for probing may be prepared from RNA preparations from cells.

Test nucleic acid may be provided from a cell as genomic DNA, cDNA or RNA, or a mixture of any of these, preferably as a library in a suitable vector. If genomic DNA is used the probe may be used to identify untranscribed regions of the gene (e.g. promoters etc.), such as is described hereinafter. Probing may optionally be done by means of so-called ‘nucleic acid chips’ (see Marshall & Hodgson (1998) Nature Biotechnology 16: 27-31, for a review).

Preliminary experiments may be performed by hybridising under low stringency conditions. For probing, preferred conditions are those which are stringent enough for there to be a simple pattern with a small number of hybridisations identified as positive which can be investigated further.

For instance, screening may initially be carried out under conditions, which comprise a temperature of about 37° C. or less, a formamide concentration of less than about 50%, and a moderate to low salt (e.g. Standard Saline Citrate (‘SSC’)=0.15 M sodium chloride; 0.15 M sodium citrate; pH 7) concentration.

Alternatively, a temperature of about 50° C. or less and a high salt (e.g. ‘SSPE’=0.180 mM sodium chloride; 9 mM disodium hydrogen phosphate; 9 mM sodium dihydrogen phosphate; 1 mM sodium EDTA; pH 7.4). Preferably the screening is carried out at about 37° C., a formamide concentration of about 20%, and a salt concentration of about 5×SSC, or a temperature of about 50° C. and a salt concentration of about 2×SSPE. These conditions will allow the identification of sequences which have a substantial degree of homology (similarity, identity) with the probe sequence, without requiring the perfect homology for the identification of a stable hybrid.

Suitable conditions include, e.g. for detection of sequences that are about 80-90% identical, hybridization overnight at 42° C. in 0.25M Na₂HPO₄, pH 7.2, 6.5% SDS, 10% dextran sulfate and a final wash at 55° C. in 0.1×SSC, 0.1% SDS. For detection of sequences that are greater than about 90% identical, suitable conditions include hybridization overnight at 65° C. in 0.25M Na₂HPO₄, pH 7.2, 6.5% SDS, 10% dextran sulfate and a final wash at 60° C. in 0.1×SSC, 0.1% SDS.

It is well known in the art to increase stringency of hybridisation gradually until only a few positive clones remain. Suitable conditions would be achieved when a large number of hybridising fragments were obtained while the background hybridisation was low.

Using these conditions nucleic acid libraries, e.g. cDNA libraries representative of expressed sequences, may be searched. Those skilled in the art are well able to employ suitable conditions of the desired stringency for selective hybridisation, taking into account factors such as oligonucleotide length and base composition, temperature and so on. One common formula for calculating the stringency conditions required to achieve hybridization between nucleic acid molecules of a specified sequence homology is (Sambrook et al., 1989):

Tₘ=81.50° C.+16.6Log [Na+]+0.41 (% G+C)−0.63 (% formamide)−600/#bp in duplex.

As an illustration of the above formula, using [Na+]=[0.368] and 50-% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. Such a sequence would be considered substantially homologous to the nucleic acid sequence of the present invention.

Binding of a probe to target nucleic acid (e.g. DNA) may be measured using any of a variety of techniques at the disposal of those skilled in the art. For instance, probes may be radioactively, fluorescently or enzymatically labelled. Other methods not employing labelling of probe include amplification using PCR (see below) or RN'ase cleavage. The identification of successful hybridisation is followed by isolation of the nucleic acid which has hybridised, which may involve one or more steps of PCR or amplification of a vector in a suitable host.

Thus one embodiment of this aspect of the present invention is nucleic acid including or consisting essentially of a sequence of nucleotides complementary to a nucleotide sequence hybridisable with any encoding sequence provided herein. Another way of looking at this would be for nucleic acid according to this aspect to be hybridisable with a nucleotide sequence complementary to any encoding sequence provided herein. Of course, DNA is generally double-stranded and blotting techniques such as Southern hybridisation are often performed following separation of the strands without a distinction being drawn between which of the strands is hybridising. Preferably the hybridisable nucleic acid or its complement encode a product able to influence a resistance characteristic of a plant, particularly an Mla-resistance response.

In a further embodiment, hybridisation of nucleic acid molecule to a variant may be determined or identified indirectly, e.g. using a nucleic acid amplification reaction, particularly the polymerase chain reaction (PCR). PCR requires the use of two primers to specifically amplify target nucleic acid, so preferably two nucleic acid molecules with sequences characteristic of Mla genes are employed. Using RACE PCR, only one such primer may be needed (see “PCR protocols; A Guide to Methods and Applications”, Eds. Innis et al, Academic Press, New York, (1990)).

PCR primers (or probes, see above) are designed based on conserved nucleotides among the Mla1 and Mla6 proven functional alleles, but not conserved among the Ma1H (=Mla1-2) sequence or any of the Morex Mla-RGH sequences. For example, forward primer: 5′TATTGTCACCGGTGCCATTC-3′, representing nt 6-26 at the N-terminus of the Mla open reading frame, can be paired with reverse primer: 5′CTCATGATGACGATTTGTGTG-3′, representing nt 2855-2875 from the C-terminus of the open reading frame (nucleotides underlined and in bold represent conserved residues among functional Mla alleles). These, and other primers based on the data, can be utilized to amplify functional alleles from lines that contain different specificities or from wild relatives. The substrate can be genomic DNA or mRNA.

Thus a method involving use of PCR in obtaining nucleic acid according to the present invention may be carried out as described above, but using a pair of nucleic acid molecule primers useful in (i.e. suitable for) PCR, at least one of which has a nucleotide sequence shown in or complementary to a sequence of an Mla1, Mla6 or Mla12 nucleic acid of the present invention (see e.g. FIGS. 3, 4, 9, 12). In each case above, if need be, clones or fragments identified in the search can be extended. For instance if it is suspected that they are incomplete, the original DNA source (e.g. a clone library, mRNA preparation etc.) can be revisited to isolate missing portions e.g. using sequences, probes or primers based on that portion which has already been obtained to identify other clones containing overlapping sequence.

The methods described above may also be used to determine the presence of one of the nucleotide sequences of the present invention within the genetic context of an individual plant, optionally a transgenic plant, which may be produced as described in more detail below. This may be useful in plant breeding programmes e.g. to directly select plants containing alleles which are responsible for desirable traits in that plant species, either in parent plants or in progeny (e.g hybrids, F1, F2 etc.). Thus use of particular novel markers defined in the Examples below, or markers which can be designed by those skilled in the art on the basis the nucleotide sequence information disclosed herein, forms one part of the present invention.

In one embodiment of this aspect, the inventors have identified as polymorphic region in intron 3 of Mla1 and Mla6 which can be used to identify functional alleles. The polymorphisms result from a simple sequence repeat (AT)ₙ. There are 14 repeats in Mla1, but only 8 (or 10) in Mla6. These findings suggest that functional Mla genes have a characteristic (AT)n repeat of varying length in intron 3.

Mla1 and Mla6 belong to a big family of NB-LRR genes. There are many Mla homologues in the barley genome and other organisms as well. Interestingly, the (AT)ₙ repeat appears to be absent in all sequence-related non-functional Mla homologues that are physically linked within the Mla complex (Wei et al., 1999) and in those searched in GENEBANK. Thus the (AT)ₙ repeat sequence may serve as a signature of functional Mla genes in the complex Mla locus. The (AT)ₙ repeat sequence may be referred to as the “micro-satellite Mla (AT)n” herein.

The finding of the Mla (AT)ₙ micro-satellite is particularly useful in view of the high degree of similarity between functional and on-functional alleles. Sequences that flank the Mla (AT)ₙ micro-satellite appear to be conserved across functional Mla genes and can serve as a basis for PCR primer design. Genomic amplification products include the Mla (AT)ₙ micro-satellite and will therefore display polymorphisms in Hordeum accessions containing known or unknown Mla resistance specificities.

Preferred primers which span the (AT)n repeat region, and can be used to tag functional Mla genes, are as follows:

| | |
|---|---|
| 1. MlaATS1 | 5′-ACTGGCATAAGCAGTTCACACTAAAC-3′ |
| 2. MlaATAS1 | 5′-CATTTATCTTCCTCTTTCCTTCCTCTCC-3′ |

Thus in one embodiment of the invention there is provided a method for isolating, identifying or locating a functional Mla allele, which includes:

(a) providing a preparation of nucleic acid from plant cells believed to encode the allele,

(b) identifying the presence of an Mla (AT)ₙ micro-satellite as described above in the nucleic acid preparation, e.g. by contacting the nucleic acid in said preparation with a probe or primer adapted to identify such a sequence,

(c) correlating the presence of an Mla (AT)ₙ micro-satellite in the preparation with the presence of a functional Mla allele.

Generally the presence of Mla (AT)ₙ micro-satellite can be most readily determined by analysis of polymorphisms in an amplified product from intron 3. Generally the sequence will include at least about 6, 8, 10, 12, 14, 16, 18, 20, 22, 24, 26, 28, 30, 32, 34, 36, 38, 40 or more AT repeats.

As with the other methods of this aspect of the invention, the Mla (AT)ₙ micro-satellite embodiment may be employed inter alia:

(i) To screen for Mla resistant transgenic plants: DNA from transgenic plants can be rapidly inspected using PCR for lines containing single or multiple Mla resistance specificities. Since each Mla specificity is likely to generate unique Mla (AT)ₙ signatures, the micro-satellite polymorphisms can serve as diagnostic tools indicating whether and how many different Mla resistance genes are present in transgenic lines.

(ii) To clone novel functional Mla genes from uncharacterized Hordeum vulgare accessions or wild relatives: the Mla (AT)ₙ micro-satellite provides a unique opportunity to screen germplasm collections for novel Mla resistance specificities that have not been used before by plant breeders. Novel Mla (AT)ₙ micro-satellite signatures are likely to indicate the presence of a novel Mla resistance specificity in a tested plant. DNA sequencing of the PCR amplicon containing the novel Mla (AT)ₙ signature should aid in developing allele-specific PCR primers that can be subsequently used to clone and sequence the corresponding full length gene by means of standard inverse PCR techniques (‘genome walker kit’, Boehringer Mannheim).

(iii) To screen for powdery mildew resistant plants or lines in conventional barley breeding programs: a major objective in plant breeding is the continuous development of novel powdery mildew resistant cultivars by introgressing new Mla resistance specificities from wild relatives (e.g. Hordeum spontaneum) into cultivated germplasm. Until now, resistant progeny in these breeding programs had to be identified by time consuming and laborious inoculation experiments involving multiple powdery mildew isolates. The availability of the Mla (AT)ₙ micro-satellite offers the opportunity to genotype plants rapidly by PCR. PCR products can be amplified with Mla microsatellite primers and can be resolved by gel electrophoresis.

As used hereinafter, unless the context demands otherwise, the term “Mla nucleic acid” is intended to cover any of the nucleic acids of the invention described above, including functional variants.

In one aspect of the present invention, the Mla nucleic acid described above is in the form of a recombinant and preferably replicable vector. “Vector” is defined to include, inter alia, any plasmid, cosmid, phage or Agrobacterium binary vector in double or single stranded linear or circular form which may or may not be self transmissible or mobilizable, and which can transform prokaryotic or eukaryotic host either by integration into the cellular genome or exist extrachromosomally (e.g. autonomous replicating plasmid with an origin of replication). Specifically included are shuttle vectors by which is meant a DNA vehicle capable, naturally or by design, of replication in two different host organisms, which may be selected from actinomycetes and related species, bacteria and eucaryotic (e.g. higher plant, mammalian, yeast or fungal cells)

A vector including nucleic acid according to the present invention need not include a promoter or other regulatory sequence, particularly if the vector is to be used to introduce the nucleic acid into cells for recombination into the genome.

Preferably the nucleic acid in the vector is under the control of, and operably linked to, an appropriate promoter or other regulatory elements for transcription in a host cell such as a microbial, e.g. bacterial, or plant cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell

By “promoter” is meant a sequence of nucleotides from which transcription may be initiated of DNA operably linked downstream (i.e. in the 3′ direction on the sense strand of double-stranded DNA). “Operably linked” means joined as part of the same nucleic acid molecule, suitably positioned and oriented for transcription to be initiated from the promoter. DNA operably linked to a promoter is “under transcriptional initiation regulation” of the promoter.

Thus this aspect of the invention provides a gene construct, preferably a replicable vector, comprising a promoter operatively linked to a nucleotide sequence provided by the present invention, such as Mla1, Mla6 or Mla12 or a variant thereof.

Generally speaking, those skilled in the art are well able to construct vectors and design protocols for recombinant gene expression. Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 2nd edition, Sambrook et al, 1989, Cold Spring Harbor Laboratory Press (or later editions of this work).

Many known techniques and protocols for manipulation of nucleic acid, for example in preparation of nucleic acid constructs, mutagenesis (see above discussion in respect of variants), sequencing, introduction of DNA into cells and gene expression, and analysis of proteins, are described in detail in Current Protocols in Molecular Biology, Second Edition, Ausubel et al. eds., John Wiley & Sons, 1992. The disclosures of Sambrook et al. and Ausubel et al. are incorporated herein by reference.

In one embodiment of this aspect of the present invention, there is provided a gene construct, preferably a replicable vector, comprising an inducible promoter operatively linked to a nucleotide sequence provided by the present invention. The term “inducible” as applied to a promoter is well understood by those skilled in the art. In essence, expression under the control of an inducible promoter is “switched on” or increased in response to an applied stimulus. The nature of the stimulus varies between promoters. Some inducible promoters cause little or undetectable levels of expression (or no expression) in the absence of the appropriate stimulus. Other inducible promoters cause detectable constitutive expression in the absence of the stimulus. Whatever the level of expression is in the absence of the stimulus, expression from any inducible promoter is increased in the presence of the correct stimulus.

Particular of interest in the present context are nucleic acid constructs which operate as plant vectors. Specific procedures and vectors previously used with wide success upon plants are described by Guerineau and Mullineaux (1993) (Plant transformation and expression vectors. In: Plant Molecular Biology Labfax (Croy RRD ed) Oxford, BIOS Scientific Publishers, pp 121-148).

Suitable promoters which operate in plants include the Cauliflower Mosaic Virus 35S (CaMV 35S). Other examples are disclosed at pg 120 of Lindsey & Jones (1989) “Plant Biotechnology in Agriculture” Pub. OU Press, Milton Keynes, UK. The promoter may be selected to include one or more sequence motifs or elements conferring developmental and/or tissue-specific regulatory control of expression. Inducible plant promoters include the ethanol induced promoter of Caddick et al (1998) Nature Biotechnology 16: 177-180.

It may be desirable to use a strong constitutive promoter.

If desired, selectable genetic markers may be included in the construct, such as those that confer selectable phenotypes such as resistance to antibiotics or herbicides (e.g. kanamycin, hygromycin, phosphinotricin, chlorsulfuron, methotrexate, gentamycin, spectinomycin, imidazolinones and glyphosate).

The present invention also provides methods comprising introduction of such a construct into a host cell, particularly a plant cell. In a further aspect of the invention, there is disclosed a host cell containing a heterologous construct according to the present invention, especially a plant or a microbial cell. The term “heterologous” is used broadly in this aspect to indicate that the gene/sequence of nucleotides in question (an Mla gene) have been introduced into said cells of the plant or an ancestor thereof, using genetic engineering, i.e. by human intervention. A heterologous gene may replace an endogenous equivalent gene, i.e. one which normally performs the same or a similar function, or the inserted sequence may be additional to the endogenous gene or other sequence.

Nucleic acid heterologous to a plant cell may be non-naturally occurring in cells of that type, variety or species. Thus the heterologous nucleic acid may comprise a coding sequence of or derived from a particular type of plant cell or species or variety of plant, placed within the context of a plant cell of a different type or species or variety of plant. A further possibility is for a nucleic acid sequence to be placed within a cell in which it or a homolog is found naturally, but wherein the nucleic acid sequence is linked and/or adjacent to nucleic acid which does not occur naturally within the cell, or cells of that type or species or variety of plant, such as operably linked to one or more regulatory sequences, such as a promoter sequence, for control of expression. The activity of Mla nucleic acid of the present invention in heterologous systems (e.g. wheat) is shown in the Examples below. The host cell (e.g. plant cell) is preferably transformed by the construct, which is to say that the construct becomes established within the cell, altering one or more of the cell's characteristics and hence phenotype e.g. with respect to powdery mildew resistance.

Nucleic acid can be transformed into plant cells using any suitable technology, such as a disarmed Ti-plasmid vector carried by Agrobacterium exploiting its natural gene transfer ability (EP-A-270355, EP-A-0116718, NAR 12(22) 8711-87215 1984), particle or microprojectile bombardment (U.S. Pat. No. 5,100,792, EP-A-444882, EP-A-434616) microinjection (WO 92/09696, WO 94/00583, EP 331083, EP 175966, Green et al. (1987) Plant Tissue and Cell Culture, Academic Press), electroporation (EP 290395, WO 8706614 Gelvin Debeyser) other forms of direct DNA uptake (DE 4005152, WO 9012096, U.S. Pat. No. 4,684,611), liposome mediated DNA uptake (e.g. Freeman et al. Plant Cell Physiol. 29: 1353 (1984)), or the vortexing method (e.g. Kindle, PNAS U.S.A. 87: 1228 (1990d) Physical methods for the transformation of plant cells are reviewed in Oard, 1991, Biotech. Adv. 9: 1-11.

Agrobacterium transformation is widely used by those skilled in the art to transform dicotyledonous species. Recently, there has also been substantial progress towards the routine production of stable, fertile transgenic plants in almost all economically relevant monocot plants (see e.g. Hiei et al. (1994) The Plant Journal 6, 271-282)). Microprojectile bombardment, electroporation and direct DNA uptake are preferred where Agrobacterium alone is inefficient or ineffective. Alternatively, a combination of different techniques may be employed to enhance the efficiency of the transformation process, eg bombardment with Agrobacterium coated microparticles (EP-A-486234) or microprojectile bombardment to induce wounding followed by co-cultivation with Agrobacterium (EP-A-486233).

The particular choice of a transformation technology will be determined by its efficiency to transform certain plant species as well as the experience and preference of the person practising the invention with a particular methodology of choice. It will be apparent to the skilled person that the particular choice of a transformation system to introduce nucleic acid into plant cells is not essential to or a limitation of the invention, nor is the choice of technique for plant regeneration. Thus a further aspect of the present invention provides a method of transforming a plant cell involving introduction of a construct as described above into a plant cell and causing or allowing recombination between the vector and the plant cell genome to introduce a nucleic acid according to the present invention into the genome.

The invention further encompasses a host cell transformed with nucleic acid or a vector according to the present invention (e.g comprising Mla1 or -6 sequence) especially a plant or a microbial cell. In the transgenic plant cell (i.e. transgenic for the nucleic acid in question) the transgene may be on an extra-genomic vector or incorporated, preferably stably, into the genome. There may be more than one heterologous nucleotide sequence per haploid genome.

Generally speaking, following transformation, a plant may be regenerated, e.g. from single cells, callus tissue or leaf discs, as is standard in the art. Almost any plant can be entirely regenerated from cells, tissues and organs of the plant. Available techniques are reviewed in Vasil et al., Cell Culture and Somatic Cell Genetics of Plants, Vol I, II and III, Laboratory Procedures and Their Applications, Academic Press, 1984, and Weissbach and Weissbach, Methods for Plant Molecular Biology, Academic Press, 1989.

The generation of fertile transgenic plants has been achieved in the cereals rice, maize, wheat, oat, and barley (reviewed in Shimamoto, K. (1994) Current Opinion in Biotechnology 5, 158-162.; Vasil, et al. (992) Bio/Technology 10, 667-674; Vain et al., 1995, Biotechnology Advances 13 (4): 653-671; Vasil, 1996, Nature Biotechnology 14 page 702).

Plants which include a plant cell according to the invention are also provided.

In addition to the regenerated plant, the present invention embraces all of the following: a clone of such a plant, selfed or hybrid progeny and descendants (e.g. F1 and F2 descendants) and any part of any of these. The invention also provides parts of such plants e.g. any part which may be used in reproduction or propagation, sexual or asexual, including cuttings, seed and so on, or which may be a commodity per se e.g. grain.

A plant according to the present invention may be one which does not breed true in one or more properties. Plant varieties may be excluded, particularly registrable plant varieties according to Plant Breeders' Rights.

The invention further provides a method of influencing or affecting the degree of resistance of a plant to a pathogen, particularly powdery mildew, more particularly to one of the isolates discussed below, the method including the step of causing or allowing expression of a heterologous nucleic acid sequence as discussed above within the cells of the plant.

The step may be preceded by the earlier step of introduction of the nucleic acid into a cell of the plant or an ancestor thereof.

The methods may also include the manipulation of other genes e.g. which may be involved in transduction of the resistance signal, or in generating a resistance response. For instance, certain Mla genes in Barley may be dependent on other genes e.g. Ral1 and/or Rar2, for resistance function (see PCT/GB99/02590 of Plant Bioscience Limited). To date, evidence indicates that mutants in barley Ral1 suppress most tested powdery mildew race-specific resistance specificities encoded at the Mla locus on chromosome 1H (Mla6, Mla9, Mla12, Mla13, Mla14, Mla22, and Mla23) as well as resistance specificities to powdery mildew at other loci (Mlat, Mlh, Mlk, Mlra, and Mlg). However, in some cases, Mla1, Mla7 and mlo, no suppression of a resistance gene function was observed (Jørgensen, 1996; Peterhansel et al. 1997).

The foregoing discussion has been generally concerned with uses of the nucleic acids of the present invention for production of functional MLA polypeptides in a plant, thereby increasing its pathogen resistance. However the information disclosed herein may also be used to reduce the activity or levels of such polypeptides in cells in which it is desired to do so. For instance the sequence information disclosed herein may be used for the down-regulation of expression of genes e.g. using anti-sense technology (see e.g. Bourque, (1995), Plant Science 105, 125-149); sense regulation [co-suppression] (see e.g. Zhang et al., (1992) The Plant Cell 4, 1575-1588). Further options for down regulation of gene expression include the use of ribozymes, e.g. hammerhead ribozymes, which can catalyse the site-specific cleavage of RNA, such as mRNA (see e.g. Jaeger (1997) “The new world of ribozymes” Curr Opin Struct Biol 7:324-335.

Nucleic acids and associated methodologies for carrying out down-regulation (e.g. complementary sequences) form one part of the present invention. The present invention also encompasses the expression product of any of the Mla (particularly functional Mla) nucleic acid sequences disclosed above, plus also methods of making the expression product by expression from encoding nucleic acid therefore under suitable conditions, which may be in suitable host cells.

A preferred polypeptide includes the amino acid sequence shown in FIG. 5, or MLA6 in FIG. 10, or MLA12 in Annex V. However a polypeptide according to the present invention may be a variant (allele, fragment, derivative, mutant or homologue etc.) of these polypeptides. The allele, variant, fragment, derivative, mutant or homologue may have substantially the Mla1, Mla12 or the Mla6 function of the amino acid sequences shown in FIG. 10 or Annex V.

Also encompassed by the present invention are polypeptides which although clearly related to a functional MLA1, MLA12 or MLA6 polypeptide (e.g. they are immunologically cross reactive with the polypeptide, or they have characteristic sequence motifs in common with the polypeptide) no longer have Mla function. Such a variant may be the polypeptide of FIG. 6, or others in FIG. 10.

Following expression, the recombinant product may, if required, be isolated from the expression system. Generally however the polypeptides of the present invention will be used in vivo (in particular in planta).

Purified MLA1, MLA12 or MLA6 or variant protein, produced recombinantly by expression from encoding nucleic acid therefor, may be used to raise antibodies employing techniques which are standard in the art. Methods of producing antibodies include immunising a mammal (e.g. mouse, rat, rabbit, horse, goat, sheep or monkey) with the protein or a fragment thereof. Antibodies may be obtained from immunised animals using any of a variety of techniques known in the art, and might be screened, preferably using binding of antibody to antigen of interest. For instance, Western blotting techniques or immunoprecipitation may be used (Armitage et al, 1992, Nature 357: 80-82). Antibodies may be polyclonal or monoclonal. As an alternative or supplement to immunising a mammal, antibodies with appropriate binding specificity may be obtained from a recombinantly produced library of expressed immunoglobulin variable domains, e.g. using lambda bacteriophage or filamentous bacteriophage which display functional immunoglobulin binding domains on their surfaces; for instance see W092/01047.

Antibodies raised to a polypeptide or peptide can be used in the identification and/or isolation of homologous polypeptides, and then the encoding genes. Thus, the present invention provides a method of identifying or isolating a polypeptide with MLA function (in accordance with embodiments disclosed herein), including screening candidate peptides or polypeptides with a polypeptide including the antigen-binding domain of an antibody (for example whole antibody or a fragment thereof) which is able to bind an MLA1, MLA12 or MLA6 peptide, polypeptide or fragment, variant or variant thereof or preferably has binding specificity for such a peptide or polypeptide, such as having an amino acid sequence identified herein. Specific binding members such as antibodies and polypeptides including antigen binding domains of antibodies that bind and are preferably specific for an MLA1, MLA12 or MLA6 peptide, or polypeptide or mutant, variant or derivative thereof represent further aspects of the present invention, as do their use and methods which employ them.

Candidate peptides or polypeptides for screening may for instance be the products of an expression library created using nucleic acid derived from an plant of interest, or may be the product of a purification process from a natural source.

In a yet further aspect of the present invention, there is disclosed a method for assessing the ability of nucleic acid encoding a putative R gene to confer resistance against a pathogen expressing a cognate Avr gene, the method comprising the steps of:

(a) selecting plant material comprising plant cells which express a recessive gene conferring resistance against the pathogen,

(b) introducing nucleic acid encoding (i) a detectable marker, (ii) a dominant susceptibility gene which inhibits the resistance conferred by the recessive gene, and (iii) the putative R gene,

(c) challenging the plant material with the pathogen,

(d) observing cells in the plant material in which the marker is expressed to determine the amount of the pathogen present, and

(e) correlating the amount of the pathogen with the ability of the R gene to confer resistance against the pathogen.

‘R’ gene and ‘Avr gene’ are used in this aspect in their art-recognised sense to represent the gene-for-gene specificity frequently displayed by plant genes which confer resistance to fungal pathogens (see For or, 1956, Phytopathology 45: 680-685 and Anderson et al, 1997, Plant Cell 9: 641-651 for a more recent review).

The use of this three-component system, in effect, reduces the background level of ‘susceptibility’ of the plant thereby reducing the likelihood that any resistance (reduced level of pathogen) conferred or otherwise by the putative R gene would be masked by the high levels of pathogen present elsewhere on the material. Thus only that material containing the marker (i.e. into which the three components are successfully introduced) is ‘susceptible’, which facilitates the observation of resistance conferred by the putative R gene.

An example of such an R/Avr interaction is that demonstrated by the race specific Mla1 gene and its cognate Avr target designated Avrlal (e.g. as encoded by the powdery mildew isolate K1). ‘Putative R gene’ in this context simply means a sequence of nucleotides which is desired to test for the requisite activity. It may be an NBS-LRR gene. There is no requirement that it be a natural, or full length, gene. It will, however, be heterologous to the plant material used in the method.

An example of a recessive gene of step (a) would be mlo gene, the effect of which is negated by the dominant susceptibility gene Mlo. The recessive gene may have broad resistance against the pathogen in question (e.g. no absolute requirement for the cognate Avr gene). This may facilitate the use of controls (see below). An example of a marker in step (b) is Green Fluorescent Protein (GFP). Another example would be GUS, or another marker described above in relation to the plant transformation aspects of the invention.

The hypothetical possibility of an assay system based on transient complementation of Mlo treated host is discussed by Shirasu et al (1999) Plant Journal 17(3), 293-299. However no actual experiments using candidate R genes were performed, and no guidance was given as to how a three-component system (rather than the two component Mlo/GFP or other marker) could be used in practice. In the present system the nucleic acid introduced in step (b) is in the form of a first vector (encoding (i) and (ii)) and a second vector (encoding (iii)) which are introduced together (e.g. by biolistic transformation) into plant material such that they are at least transiently expressed therein.

Step (c) can be by any method commonly used in the art. In principle the pathogen need not be the natural pathogen, but could be any transformed or transgenic cell or organism which expresses the appropriate Avr gene and which can invade the plant material. The observation in step (d) can be direct or otherwise. The amount in this case can mean simply presence or absence; it does not imply the requirement for accurate quantification.

Preferably for step (e) the amount is compared against a corresponding control system in which either (1) no R gene is present, or (2) the pathogen does not express a cognate Avr gene, but one which is still recognised by the recessive gene. In each case more pathogen would be expected (on the ‘marked’ material) than in the successful case when an R gene is expressed in the presence of a pathogen expressing its cognate Avr gene.

The method above can also be used, correspondingly, to identify pathogens expressing cognate Avr genes for known R genes, and also inhibitors of this interaction.

Vectors for use in step (b), particularly a first vector encoding (i) a detectable marker, (ii) a dominant susceptibility gene which inhibits the resistance conferred by the recessive gene, form a further aspect of the present invention, as does their use in all or part of the method described above. An example vector is pUGLUM in Example 5 below.

The above description has generally been concerned with the translated and coding parts of Mla genes. Also embraced within the present invention are untranscribed parts (UTRs) of the genes. Thus a further aspect of the invention is an isolated nucleic acid molecule encoding the promoter, or other UTR (3′ or 5′ ), of an Mla gene. Promoter and UTR sequences are shown within the Figures and Annexes below.

Also embraced by the present invention is a promoter which is a mutant, derivative, or other homolog of an Mla promoter. These can be generated or identified as described above; they will share homology with the Mla promoter and retain promoter activity. “Promoter activity” is used to refer to ability to initiate transcription. The level of promoter activity is quantifiable for instance by assessment of the amount of mRNA produced by transcription from the promoter or by assessment of the amount of protein product produced by translation of mRNA produced by transcription from the promoter. The amount of a specific mRNA present in an expression system may be determined for example using specific oligonucleotides which are able to hybridise with the mRNA and which are labelled or may be used in a specific amplification reaction such as the polymerase chain reaction.

To find minimal elements or motifs responsible for promoter activity, or particular regulatory control elements, restriction enzyme or nucleases may be used to digest a nucleic acid molecule, or mutagenesis may be employed, followed by an appropriate assay (for example using a reporter gene such as luciferase) to determine the sequence required. Nucleic acid comprising these elements or motifs forms one part of the present invention.

The invention will now be further described with reference to the following non-limiting Figures and Examples. Other embodiments of the invention will occur to those skilled in the art in the light of these.

### FIGURES, TABLES, SEQUENCE

FIG. 1: a three-component single-cell functional assay system based on Mlo, GFP, and Mla1, as described in Example 5.

FIG. 2: vector pUGLUM, a 9.8 kb plasmid harbouring both GFP and Mlo each driven by the ubiquitin 1 promoter, as described in Example 5.

FIGS. 3 and 4: two genes designated herein R gene A (Mla1 Gene Sequence) and B (Mla1 Gene Homologue Sequence) obtained from cosmid p6-49-2-15 and p6-49-2-7 The genes showing significant sequence-relatedness to NBS-LRR type R genes. The bold letters represent exon sequences.

FIGS. 5 and 6: conceptual protein sequences based on gene A and gene B of 958 and 949 amino acids respectively.

FIG. 7: Selection scheme for complementation of Mla6 specificity. (A) Genomic DNA of the Mla6-containing line, C.I. 16151, was used as template to amplify the LRR encoding regions of RGH1a, RGH1e, RGH2a, and RGH3a (see Methods). (B) RGH family specific probes were used individually to hybridize 400,000 pfu of a C.I. 16151 lambda-ZapII cDNA library. Twenty-nine NBS-LRR encoding cDNAs were identified with the RGH1a/RGH1e probe. (C) The Mla6-cosegregating, C.I. 16151 cDNA sequence was used to design PCR primers to screen super pools of a 3-genome equivalent C.I. 16151 cosmid library. Individual cosmid clones were purified from the identified pools, fingerprinted by restriction digestion, and confirmed via hybridization to the candidate cDNA. (D) Cosmid 9589-5a was used, to complement AvrMla6-dependent resistance specificity via the 3-component single-cell assay.

Distances are in centimorgans across the top horizontal line and YACs/BACs in the 400-kb contig are drawn to scale in kilobases below. The Franka YAC is designated by a “Fr” prefix, whereas, the Morex BACs are designated by a “Mo” prefix. A filled-in circle designates that YAC/BAC was amplified by the respective end-clone primer set or it hybridized to the amplified product. Horizontal arrowheads designate the T7 side of the BAC vector. Locations of Morex RGH sequences are designated by vertical rectangles. RGH1 sequences are designated as shaded, RGH2 as white, and RGH3 as diagonal hash marks within the rectangle.

FIG. 8: Three classes of candidate Mla6 transcripts.(A) Representation of the 5′ untranslated regions of the 3 Mla6 cDNA classes. Black arrowheads indicate the position of the 17-nt repeat in class A (Mla6). Classes B and C (Mla6-2) differ only by the presence or absence of introns 1 and 2 and are divergent from Mla6 but identical to Mla1 near the 5′ end. All cDNAs encode a small 9 amino acid peptide (uORF) located before the first putative 5′ UTR intron (designated by red arrowheads). An identical peptide is encoded within the 5′ UTR of Mla1 (Zhou et al., in press). The 3′ end of this uORF spans the first intron-splicing site. The presence of this intron, as in classes A and B, results in the addition of only one amino acid to the uORF because of a stop codon existing very early in the intron. The genomic sequence of Mla6 was obtained from cosmid 9589-5a that was shown to be functional in the 3-component transient assay. (B) Representation of the open reading frames (including introns) encoded by Mla6 and Mla6-2. The open reading frame of Mla6 contains two introns, 992-nt and 113-nt in length. The open reading frame of Mla6-2 is nearly identical to Mla6 up to 584-nt downstream of the AUG start codon. The 4 divergent bases within this region are designated with arrows. An insertion at base 250 causes a frame-shift leading to an early stop codon in Mla6-2. The remaining 79 bases have no significant similarity to Mla6. As used herein, Mla6 is used synonymously with Mla6A, unless context demands otherwise.

FIG. 9: nucleotide sequence alignment of Mla6, Mla1, Mla1 homologue (also termed Mla1-2 herein), and four Mla-RGH1 family members from the barley cultivar Morex (Wei et al., 1999). Shaded boxes indicate identical residues.

FIG. 10: amino acid sequence alignment of MLA6, MLA1, MLA1-homologue (also termed MLA1-2 herein), and four MLA-RGH1 family members from the barley cultivar Morex (Wei et al., 1999). Shaded boxes indicate similar residues. Conserved motifs within the NBS region are indicated above the sequence. The stars denote the putative solvent exposed residues of the LRR region. The carets indicate residues conserved between MLA6 and MLA1 but not with any other protein. RGH1e and RGH1f gene sequences differ by only one nucleotide, which does not cause an amino acid change. Note the presence of a premature stop codon at position 151 of these two classes. A large deletion starting at position 114 of RGH1bcd causes a frameshift mutation. The homologous frame is shown in the alignment after this deletion.

FIG. 11: Mla1, Mla6 and Mla12—alignment of genomic sequences.

Table 1: 12 cosmids isolated from the library representing genomic DNA from cultivar AlgR Mla1 (see Example 4).

Table 2: testing for the presence of Mla1 in cosmid clones—results obtained upon transfection of pUGLUM only, pUGLUM co-bombarded with cosmid p7-35-2, and cosmid p6-49-2.

Table 3: testing the function of R genes A and B separately by transient expression in detached leaves by co-bombardment of each subclone together with pUGLUM. A 15 kb EcoRI subclone containing only R gene A is designated p6-49-2-15. A 7 kb DraI subclone containing only R gene B is designated p6-49-2-7.

Table 4: various RGH-specific primer pairs utilized for obtaining probes for cDNA library screening. The Mla6 specific primers shown in the Table were used to screen pools of 10,000 cosmids each via PCR. Cosmids were purified from these 7 identified pools via colony hybridisation.

Table 5:

(a) results of bombardment of mlo-5 barley leaves with pUGLUM, pUGLUM and Mla6 cosmid 9589-5a, or pUGLUM and a Mla1 cosmid. Bombarded leaves were inoculated with either B. graminis isolate A6 or K1.

(b) Results of bombardment of mlo-5/rar1 barley leaves with pUGLUM, pUGLUM and Mla6 cosmid 9589-5a, or pUGLUM and a Mla1 cosmid. Bombarded leaves were inoculated with B. graminis isolate A6.

(c) Results of bombardment of wheat leaves with pUGUS and Mla6 cosmid 9589-5a, or pUGUS and a Mla1 cosmid. Bombarded leaves were inoculated with either B. graminis f. sp. hordei isolate A6 or B. graminis f. sp. tritici isolate JIW48.

Table 6: gene-specific primers for PCR and sequencing of Mla12 from mutants.

Sequences

Annex I—Mla6 ORF

Annex II—Mla6 cDNA

Annex III—Mla6 gDNA

Annex IV—Mla12 cDNA

Annex V—Mla12 polypeptide

### EXAMPLES

### Example 1

### characterisation of Mla1 Mutant Lines

A collection of 28 mutants derived from a Mla1 resistant barley line was kindly provided as M4 seeds by Dr. S. Somerville. The mutants were generated either by sodium azide treatment or γ-ray irradiation of barley line CI-16137 (AlgR Mla1) and identified after screens for altered phenotypes upon inoculation with Erysiphe graminis f sp hordei race CR3 containing AvrMla1. To test the provided mutant material for susceptibility against another powdery mildew isolate containing AvrMla1, inoculation experiments were performed with fungal isolate CC1 (provided by Dr. J. K. M. Brown, The John Innes Centre, U.K.) by using detached leaves of each mutant line. Four mutant lines (M516, M518, M557, and M558) showed a resistant phenotype in comparison to the susceptible (AlgS) and resistant control (AlgR Mla1). The other 24 mutant lines showed increased fungal mycelia growth compared to wild-type AlgR Mla1.

To test whether the susceptible lines contain the genetic background of the wild-type line AlgR Mla1 and do not represent seed contaminations, a set of specific markers (Y10, AE13, b6) were employed. Barley lines AlgR Mla1 and AlgS differ only by an introgressed fragment containing Mla1 (Mosemann, 1972). Markers Y10 and AE13 reside 0.62 cM and 2.6 cM distal (telomeric) to Mla, respectively. Both markers are located within the introgressed fragment of AlgR and polymorphic compared to AlgS. Marker information for Y10 and AE13 for PCR screenings of the mutant lines was kindly provided by S. Somerville. In addition, genomic Southern hybridisation of R gene homologue b6 (which maps 0.65 cM telomeric of Mla; Wei et al., 1999) can be used to detect a DNA polymorphism between AlgR Mla1 and AlgS. PCR analysis using Y10 and AE13 markers was performed on the mutants and Southern hybridisations using the b6 probe were carried out (data not shown). Two mutant lines were found (M529 and M537) that carried at least one flanking marker allele of the susceptible line AlgS and thus are not genuine mutant lines derived from the AlgR Mla1 resistant line. Therefore, their susceptible phenotype compared to AlgR Mla1 can be explained by heterozygosity at Mla and not necessarily by disruption of the Mla1 gene after mutagenesis.

Taken together, the essential result of the molecular analysis of 28 susceptible candidate Mla1 mutants identified 22 genuine mutants. These mutants were confirmed by phenotypic analysis for Mla1-specified resistance and by DNA fingerprinting with three markers tightly linked to the Mla locus.

### Example 2

### Screening Mla1 Mutants with NBS-LRR Candidate Genes

A systematic survey of the 22 remaining genuine mutants was carried out to try and detect Mla1. Radiation-induced mutagenesis has been shown to induce deletions and other chromosomal rearrangements in plant genomes and can be used to identify genes in molecular approaches (Shirley et al., 1992). Although the PCR primers designed to amplify NBS-LRR gene fragments detect only a small proportion of the gene, they might still detect deletions or rearrangements that include the amplified sequence and can therefore be scored as a presence/absence-polymorphism of PCR products. The large number of 22 mutants further supported the assumption that at least one mutant would exhibit a mutation-induced DNA polymorphism detectable by specific amplification primers.

A first screening approach was based on PCR using specific primers derived from each of four NBS-LRR genes on BAC80H14 (see Wei et al., 1999) in an attempt to amplify DNA from Mla resistant barley lines. This approach was speculative because the DNA of this BAC was derived from barley cultivar Morex which does not contain a characterised Mla specificity and therefore it could not be judged whether the RGHs shared appropriate DNA sequence similarity to Mla1 resistant and other susceptible lines, and therefore whether they could be utilised to amplify NBS-LRR homologues from AlgR Mla1 and AlgS.

PCR with NBS-LRR gene primers was first employed with different Mla backcross lines to test for specific amplification of four candidate homologues in backgrounds with different Mla specificities. A PCR product for RGH3a could be amplified from several Mla backcross lines including the Mla1 resistant line AlgR but showed no polymorphism in all tested Mla1 mutants. The PCR amplification for the other homologues revealed a surprising divergence between Morex and several Mla backcross lines. RGH1e could only be amplified from backcross lines containing Mla1 but not from accessions carrying Mla6, Mla12, Mla13 tor Mla14 (data not shown). RGH1b could only be amplified from DNA of cultivar Morex but no other backcross line.

The lack of amplification products using different Mla backcross line DNAs as template in the PCR analysis indicated a surprising sequence divergence and/or copy number variability for RGHs at Mla. This made it difficult to screen for candidate genes based on specific PCR primers of each R gene homologue. This problem could be avoided by Southern analysis on mutant filters carrying DNA from different mutant lines using the RGH gene fragments as hybridisation probes. In contrast to PCR reactions which require a high DNA sequence similarity within short stretches of the priming sites, cross-hybridization in Southern analysis would be achieved with a threshold nucleotide sequence identity as low as 70% over the complete probe length (Sambrook et al., 1992).

Southern-hybridisations were performed with RGH3a, 1bcd, 2a, and 1e. A range of four different restriction digests (HindIII, EcoRI, HaeIII, and AluI) were employed with DNA of AlgR Mla1 and mutant lines to prepare a set of mutant filters. All four RGH gene probes detected distinct hybridisation patterns, but no polymorphism was seen in any of the tested mutants. Therefore, either the mutation events are not detectable by the employed experiments or none of the tested R gene probes detected the Mla1 gene.

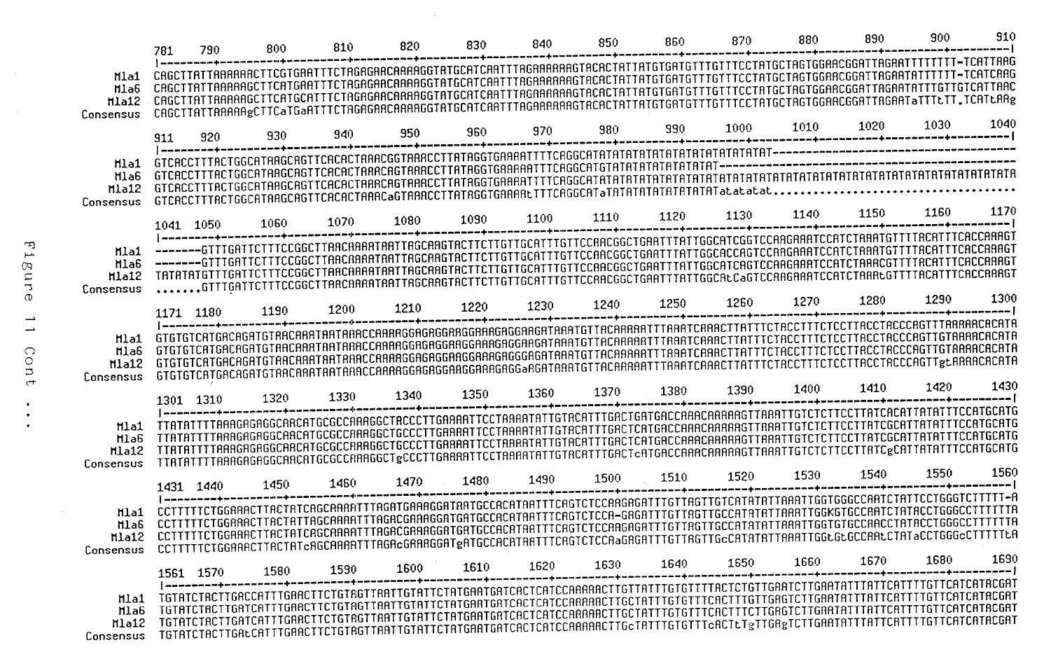

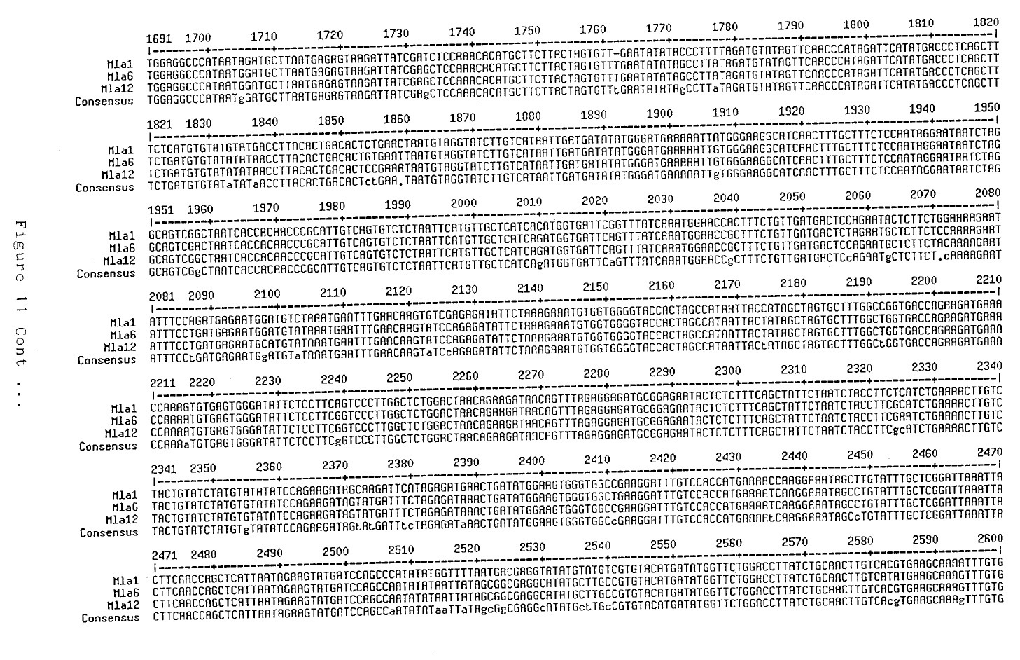

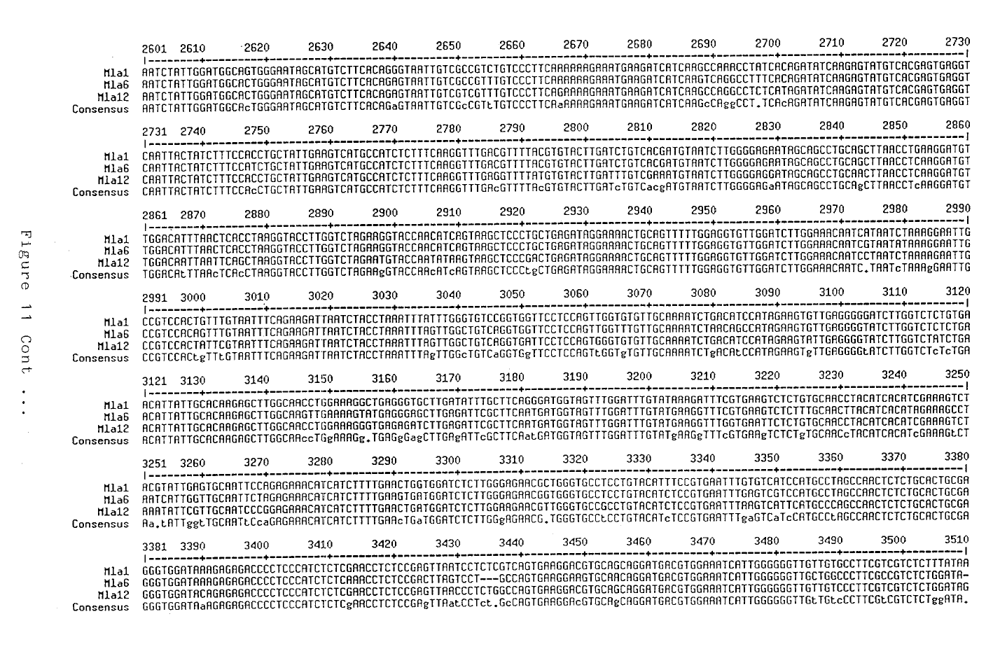

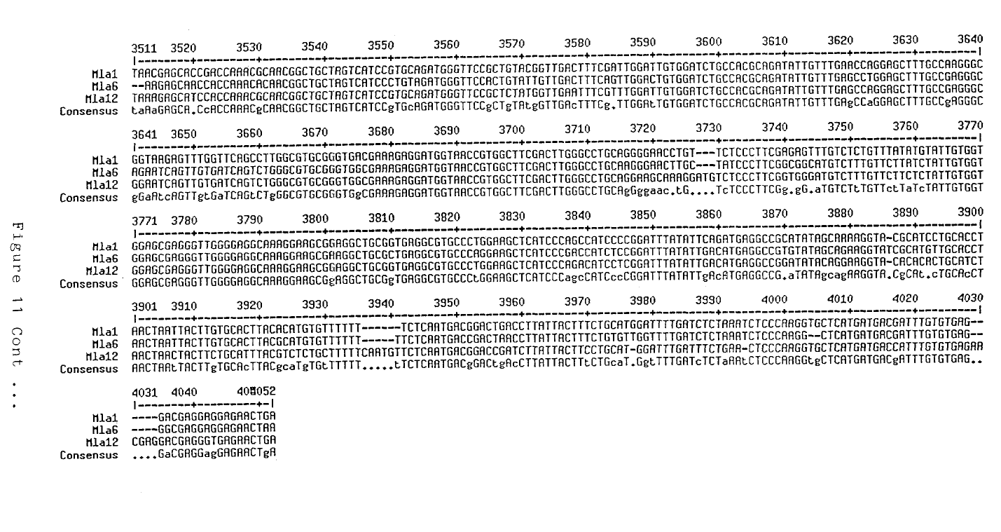

The screen for mutation-induced polymorphisms was extended to two more probes. RFLP probe MWG2083 and MWG2197 (see FIG. 7) were used on the mutant filters in Southern hybridizations. All mutant lines showed the same hybridisation pattern as the resistant parent except M508 and M510, which revealed a mutation-induced deletion. This was interpreted as first evidence that in two independent mutant lines a part of the Mla locus and sequences in direction of the telomere had been disrupted by deletion events.

Next we used primers for RGH1a to generate a 582 bp hybridization probe from cultivar Morex. These primers (39F13 and 39B95) are shown in Table 4.

RGH1a detected a major band and at least two minor bands on blots with HindIII digested DNA of AlgR Mla1. The two mutant lines M508 and M510 show complete absence of one of the two minor bands (not shown). This suggests that the deleted area in the mutant lines contains at least one R gene homologue with sufficient nucleotide sequence similarity to RGH1a detected by cross-hybridization. Since none of the tested RGH probes telomeric from and including RGH1bcd detected polymorphisms in lines 508 and 510, the data suggest that the mutation-induced deletions do not extend across RGH1bcd but disrupt only a small part of the Mla locus. However, due to the diversity of NBS-LRR genes at Mla (see above), it cannot be concluded that the Mla1 resistant line contains the same copy number and physical organisation of RGH1a or other R genes compared to BAC80H14 which is derived from a cultivar lacking a characterised Mla resistance specificity.

Provided that there is physical and genetical colinearity at Mla for the investigated barley accessions carrying no or different Mla resistance specificities, Mla1 appears to be physically delimited between the loci RGH1bcd and b6 and is further genetically delimited in the Mla high resolution map by RFLP marker MWG2197 (see Wei et al, 1999, based on Morex). This would indicate that Mla1 is physically delimited to a maximum of 170 kb as the two closest markers (MWG2197 and RGH1b) are present on overlapping YAC120 and BAC80H14, respectively (FIG. 7). Such an assumption is consistent with observations in a high resolution genetic map of Mla1: MWG2197 and Mla1 have been separated by two recombination events in a population consisting of 932 tested F2 progeny segregating for Mla1 (Schwarz et al., 1999).

### Example 3

### Construction of a Cosmid Genomic DNA Library From Cultivar AlgR Mla1 & Functional Assay

A cosmid library was constructed from barley cultivar AlgR Mla1. According to the manufacturer's instructions, the SuperCos1 vector (Stratagene) was first linearised with XbaI between the cosmid sites, and subsequently two cosmid arms were released by a BamHI digest. This generated cosmid fragments of 1.1 and 6.5 kb and an aliquot was size-fractionated by agarose gel electrophoresis to test for complete digestion. Genomic barley DNA was partially digested with MboI to result in fragment sizes of 30 to 50 kb and the termini were subsequently dephosphorylated. After ligation and packaging into cosmid particles using the Gigapack XLIII kit (Stratagene), the resulting library was titered to test for efficiency of library construction. In total seven packaging reactions were performed to obtain approximately 1,680,000 cosmid clones. Assuming an average insert size of ˜40 kb in a single recombinant cosmid this equals 67.2×10⁹ bp, representing more than 11 times the haploid barley genome size (5.3×10⁹ bp; Bennett and Smith, 1991). Plasmid DNA was isolated from 18 randomly chosen cosmid clones and analysed by agarose gel electrophoresis following NotI and EcoRI restriction digests. All 18 randomly chosen clones contained inserts with sizes ranging from between 35 and 45 kb as expected.

For a systematic screening with markers at Mla, E. coli carrying cosmids were grown at a density of ˜4,000 individual colonies per plate. Colonies were then washed from the plate with LB media and collected in Eppendorf reaction tubes. Each tube therefore represented a pool of 4,000 clones and for each of the seven packaging reactions 60 pools were collected. Two aliquots of each pool were stored as bacterial stock in glycerol at ˜70° C. and were subsequently used as template for colony hybridisation experiments. 1.5 ml of each pool was used for plasmid DNA preparation and served as template for PCR-based screenings of the library.

### Example 4

### Isolation of Cosmid Clones From the Mla Locus Harbouring Mla1

A number of cosmid clones were isolated from the cosmid library representing genomic DNA from cultivar AlgR Mla1. Cosmids were isolated by screening the library consecutively with probes previously shown to map at or close to the Morex Mla locus (see Wei et al., 1999). These probes were RGH1a, RGH1bcd, an approximately 1 kb probe derived from the proximal (centromeric) end of Morex BAC80H14 (designated B2), as well as RFLP markers MWG2083 and MWG2197. A total of 12 cosmids were isolated from the library and are listed in Table 1. Interestingly, the two cosmids isolated with probe B2 also hybridized with probe RGH1a, whereas four other cosmids hybridized only with RGH1a. This suggested the presence of multiple sequence-related copies that cross-hybridize with RGH1a in the AlgR Mla1 genotype.

DNA fingerprinting of the four cosmids identified with probe RGH1a revealed different restriction enzyme patterns for each clone. Only one of these, P6-49-2, contained a HindIII fragment, cross-hybridizing with RGH1a, that was of identical size to the one deleted in mutants M508 and M510 (see above). This was interpreted as first evidence that cosmid P6-49-2 represents a genomic segment harbouring at least one R gene homologue deleted in two of the Mla1 mutants.

### Example 5

### Development of a Transient Single-cell Expression System to Identify Genomic Cosmid Clones Encoding Mla1

In view of the limited information gathered from the Mla1 mutant survey and the unexpected diversity of RGHs in congenic lines harboring different Mla specificities, it was decided to perform a functional assay to test directly several R gene candidates from AlgR Mla1 for their function. However published technologies for generating transgenic barley plants are insufficient to test large numbers of candidate genes, distributed over an area of at least 240 kb, in a short time scale. Accordingly a novel, rapid, functional assay method was developed that would enable us to test rapidly large genomic DNA fragments, with a size typically found in recombinant cosmid clones, for the presence of genes mediating race-specific powdery mildew resistance.

The test is based on the observation that resistance mediated by Mla1 is activated rapidly after fungal attack. Mla1 resistance is usually manifested as a single-cell event, i.e. an attempted infection from a fungal germling expressing AvrMla1 is arrested in an attacked single epidermal host cell. Many attacked epidermal cells activate a suicide response, frequently termed the hypersensitive response (HR). The activated Mla1 resistance is highly effective, enabling only in exceptional cases the growth of sparse aerial hyphae at single plant-fungus interaction sites.

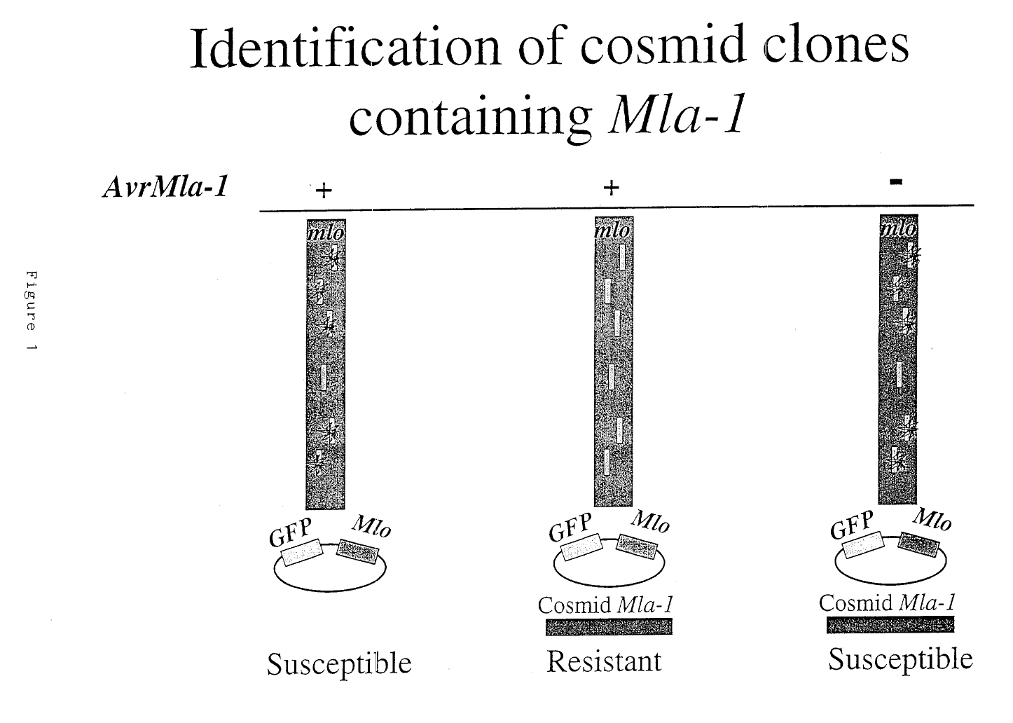

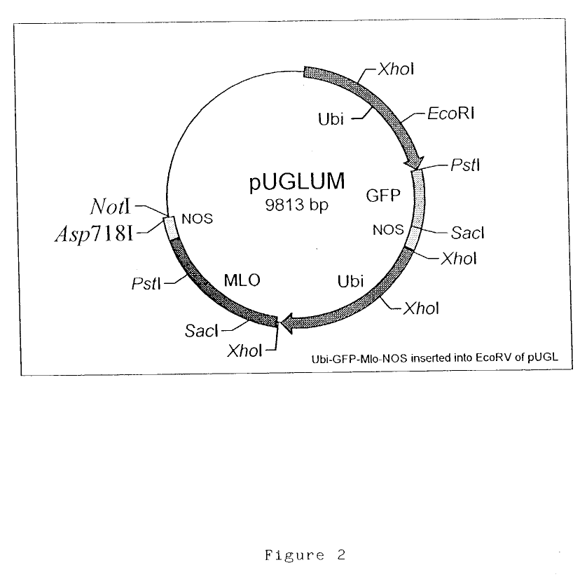

We have previously reported a biolistic transient expression system that was used to demonstrate a cell-autonomous complementation of broad-spectrum powdery mildew resistance controlled by recessive mlo alleles by transfection of the Mlo wild type gene (Shirasu et al., 1999). Mlo was transiently expressed with a marker gene (GFP) encoding a modified green fluorescent protein in single leaf epidermal cells of mlo resistant barley. Fungal inoculation of epidermal cells transfected with wild-type Mlo led to haustorium development and abundant sporulation. Complementation of mlo resistance alleles was restricted to single host epidermal cells, indicating a cell-autonomous function for the wild-type Mlo protein. We reasoned that co-expression of Mlo and cosmid clones harbouring Mla1 would compromise colony formation in Mlo transfected epidermal cells only if challenged with a fungal isolate carrying the cognate avirulence gene (AvrMla1). If, however, the transfected cells were challenged with a fungal isolate lacking AvrMla1, unrestricted growth of powdery mildew colonies would be expected (FIG. 1). Formally, this represents a three-component single-cell assay system (Mlo, GFP, and Mla1).

Towards this objective, we first modified DNA vectors for the transfection assays. This was essential because previous experiments were based on a co-bombardment of two separate plasmid vectors encoding GFP and Mlo (Shirasu et al., 1999). To obtain statistically significant numbers of single host cells expressing simultaneously GFP, Mlo, and Mla1, we constructed vector pUGLUM, a 9.8 kb plasmid harbouring both GFP and Mlo each driven by the ubiquitin 1 promoter (FIG. 2).

The pUGLUM vector was created by modifying the vector pU-hGFP-C3-N (Shirasu et al., 1999) to contain a second maize Ubiquitin promoter and the barley Mlo cDNA followed by the Nopaline synthase terminator sequence (Nos). pU-hGFP-C3-N was partially digested with EcoRI and a linker containing EcoRV Asp718 and NotI encoded by the following oligonucleotides was inserted: EcoRVKN1 (5′-AATTCGATATCGGTACCAAGCGGCCGCG) EcoRVKN2 (5′-AATTCGCCGCCGCTTGGATCCGATATCG) to create pUGL. The second Ubiquitin promoter was created by PCR amplification using the following primers: Ubi1 (5′-TAATGAGC-ATTGCATGTCTAAG and Ubi2 (5′-TGCAGAAGTAACACCAAAC-AAC) and was cloned into pGEMT (Promega) for confirmation by sequencing. The promoter was released by digestion with SacII and NotI, blunt-ended using the Klenow fragment and cloned into the EcoRV site of the modified pU-hGFP-C3-N. The Mlo cDNA (Bueschges et al. 1997) was cloned into a pBluescript KS+ vector containing the Nos terminator, and the Mlo-Nos fragment was released with Asp718 and NotI and cloned into PUGLU to create PUGLUM.

Co-bombardment experiments were then carried out with pUGLUM and candidate cosmids representing different intervals of the Mla locus. Detached leaves of cultivar BC Ingrid mlo-5 Mla-8 were used for the transfection experiments following protocols described previously by Shirasu et al., 1999. To test for the presence of Mla1 in the candidate cosmids we inoculated one half of the transfected leaves (usually eight out of a total of 16) with powdery mildew isolate k1 (AvrMla1) and challenged the other half with isolate A6 lacking AvrMla1.

Next we performed a series of transient expression experiments to test for the presence of Mla1 in several cosmid clones listed in Table 1. A representative example of results obtained upon transfection of PUGLUM only, pUGLUM co-bombarded with cosmid p7-35-2 and cosmid p6-49-2 is shown in Table 2. Leaves challenged with powdery mildew isolate A6 and transfected either with cosmid p7-35-2 or p6-49-2 resulted in a comparable number of sporulating powdery mildew colonies (42 and 29, respectively). K1 and A6 challenge supported also growth of a comparable number of powdery mildew colonies on leaves transfected with p7-35-2 (42 and 31, respectively). In contrast, p6-49-2 transfected leaves displayed a significantly lower number of colonies upon inoculation with isolate K1 compared to an A6 challenge (5 and 29 colonies, respectively). These data suggested the presence of a gene in cosmid p6-49-2 mediating growth arrest only of the fungal isolate containing AvrMla1. Interestingly, the number of detectable GFP expressing cells appeared to be lower in p6-49-2 transfected leaves following K1 inoculation in comparison to an A6 challenge (24 and 66, respectively). If p6-49-2 contains Mla1, then one could explain this observation with the frequent activation of an HR cell death in response to pathogen challenge, in consequence inactivating possibly the GFP marker protein. No other transfected cosmid tested by the above described protocol mediated a differential phenotype upon A6 and k1 spore inoculation or provided evidence for. enhanced fungal resistance to both isolates (data not shown).

### Example 6

### Sequencing of Cosmid p6-49-2

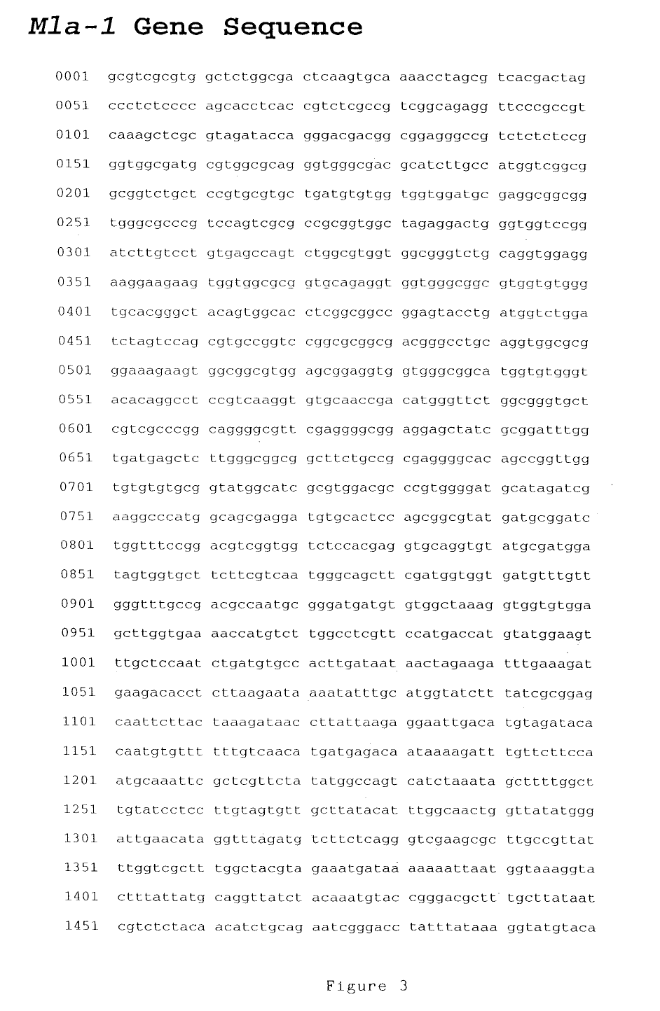

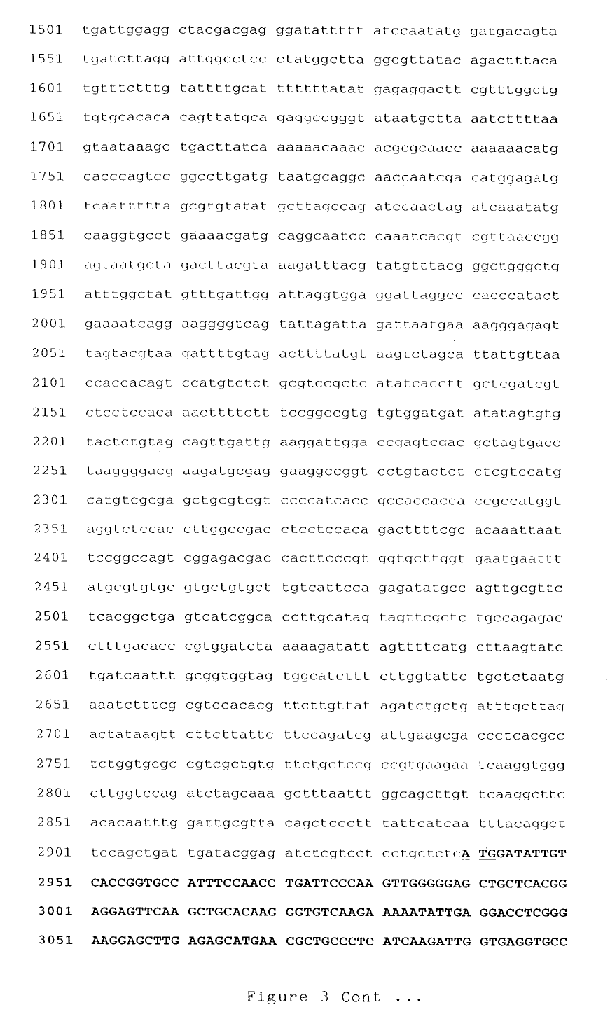

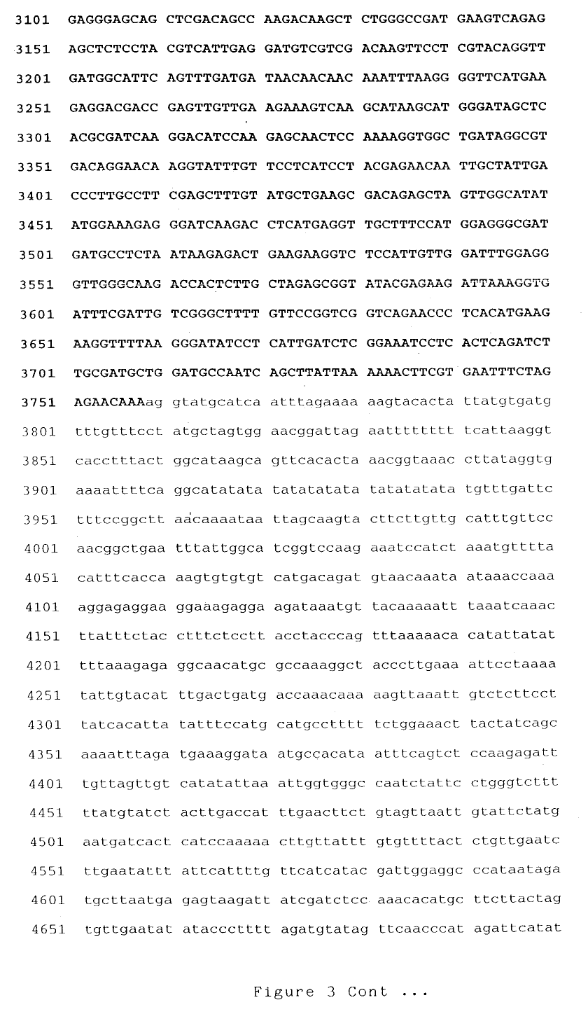

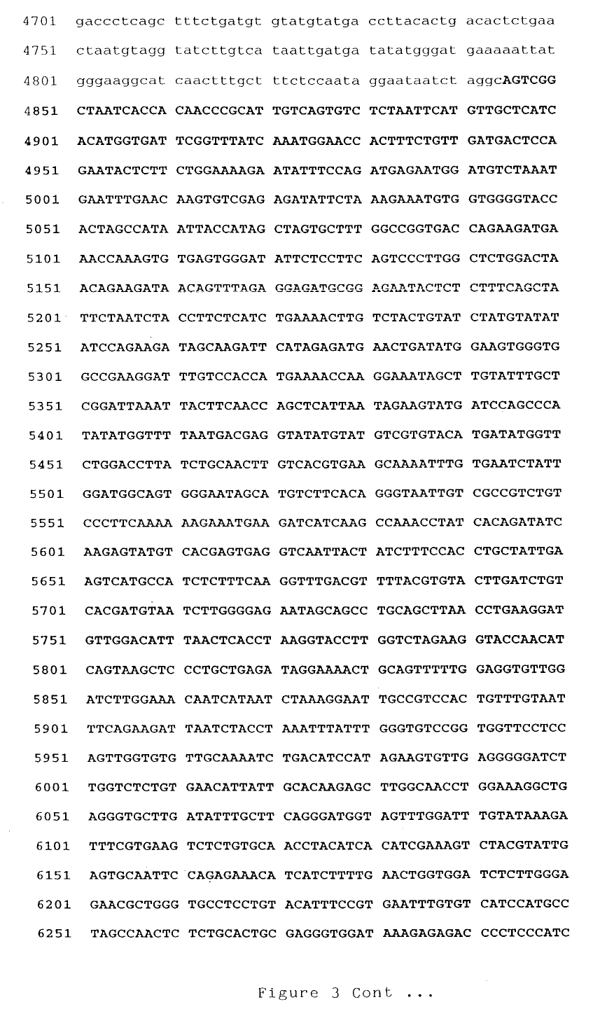

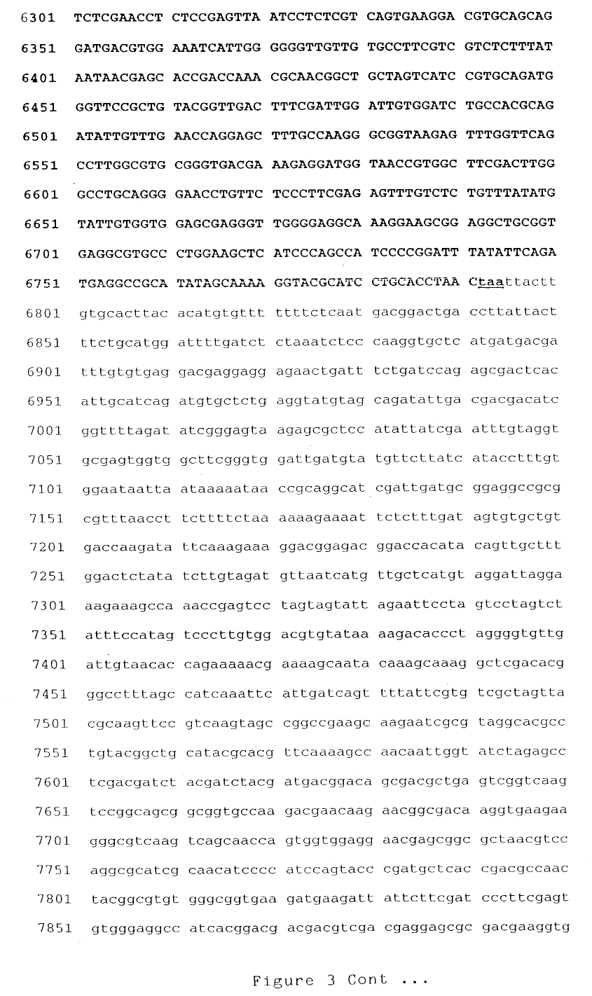

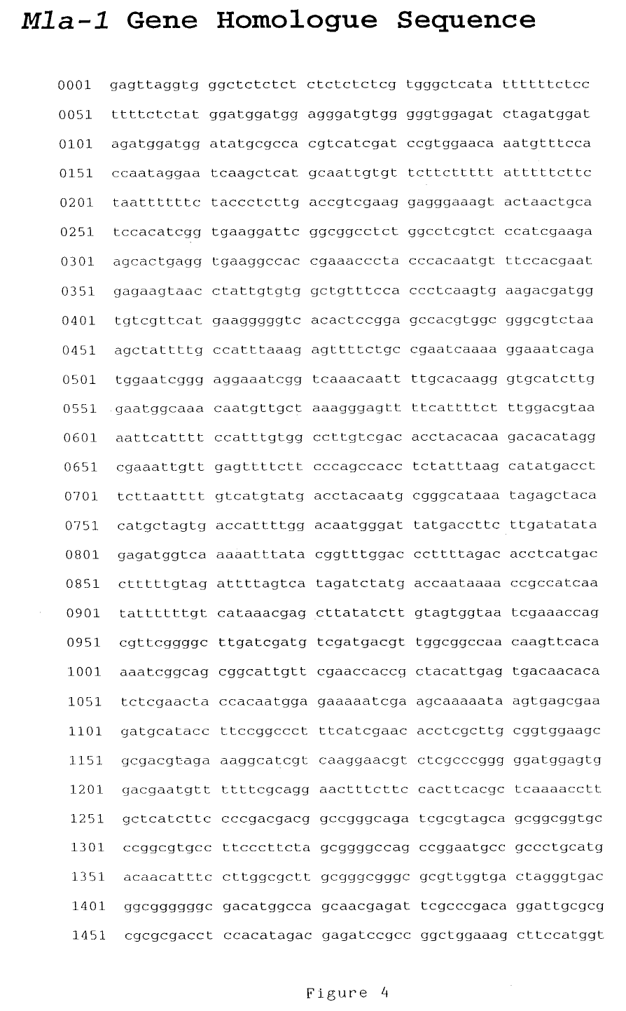

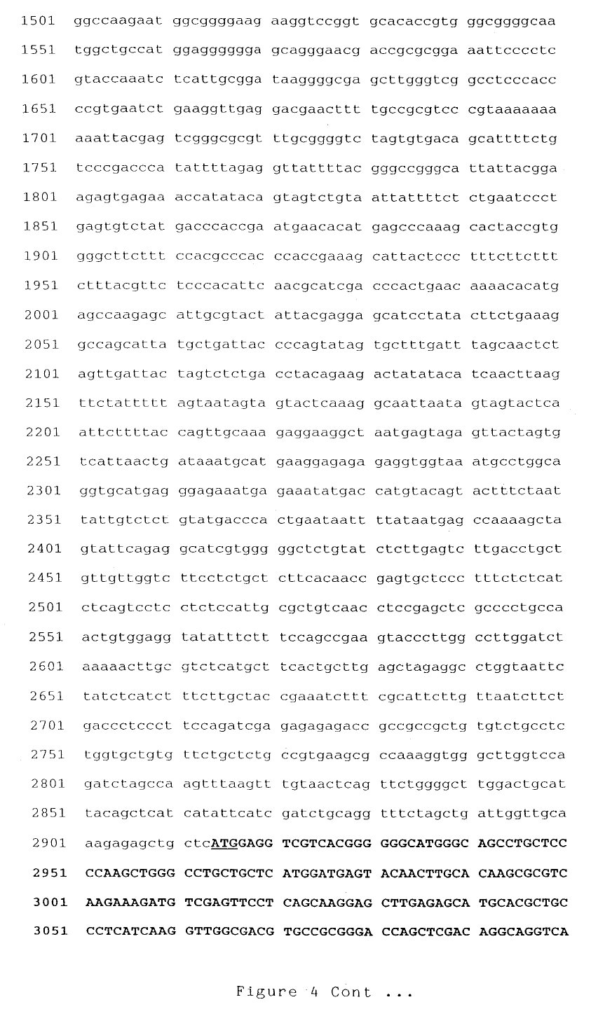

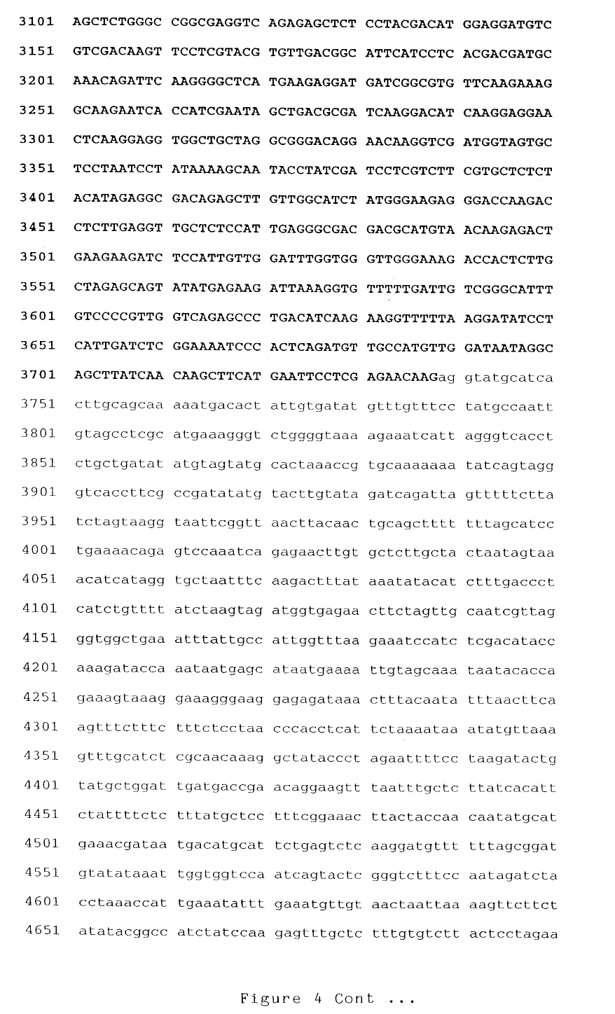

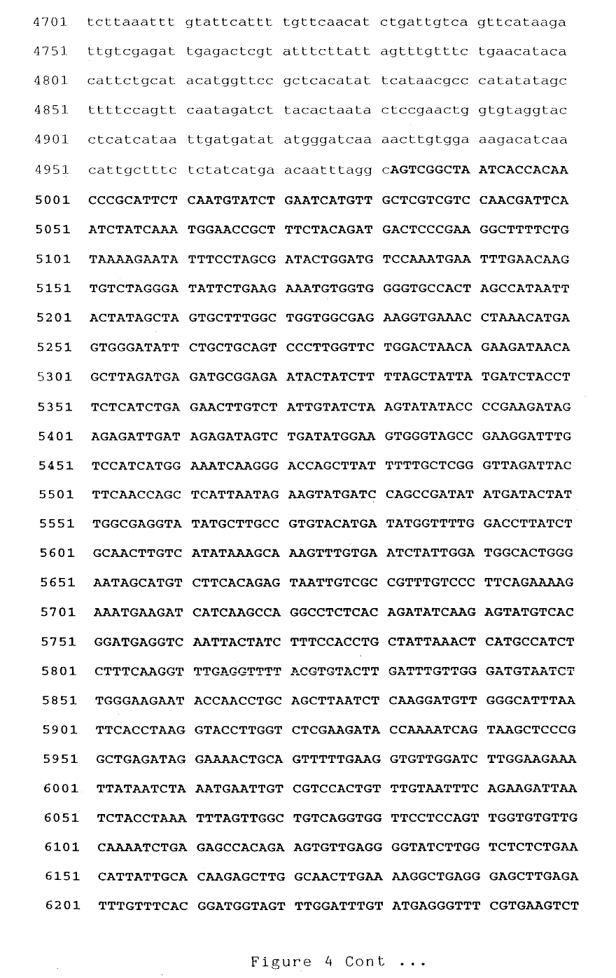

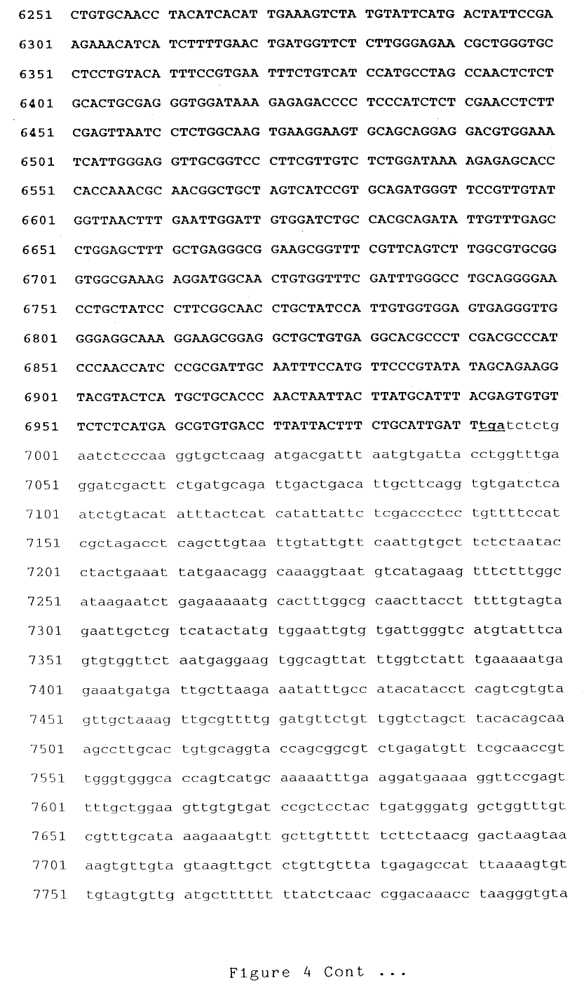

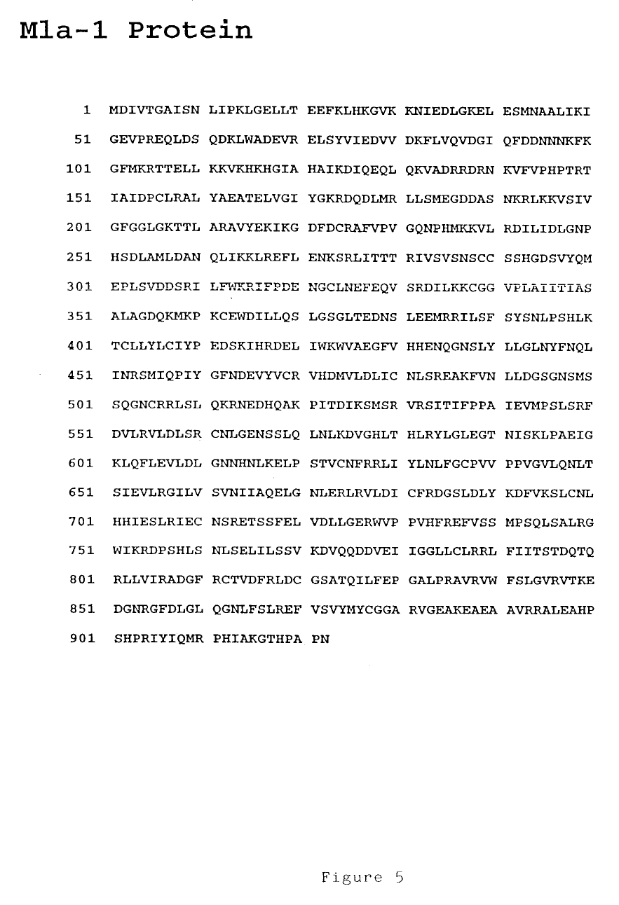

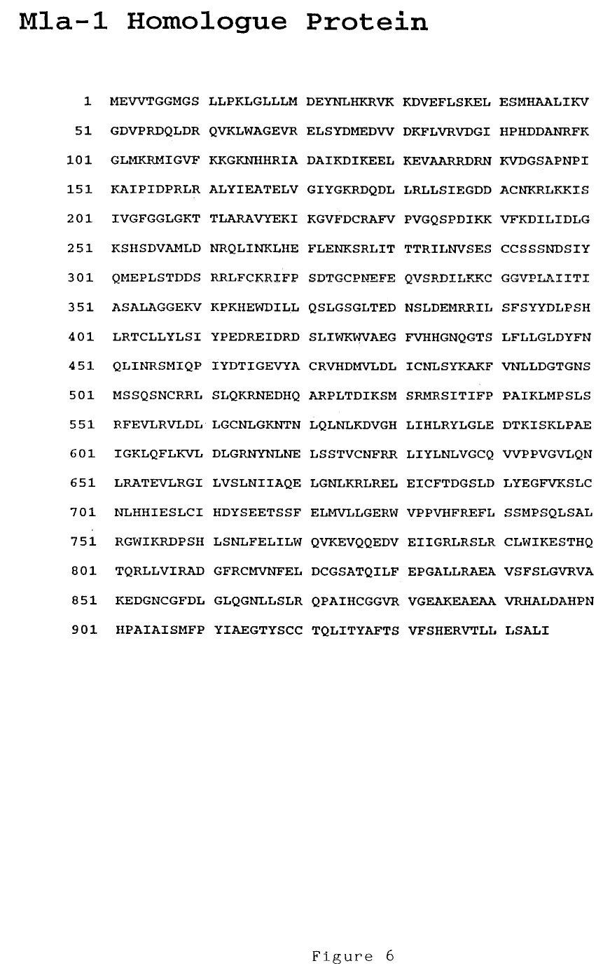

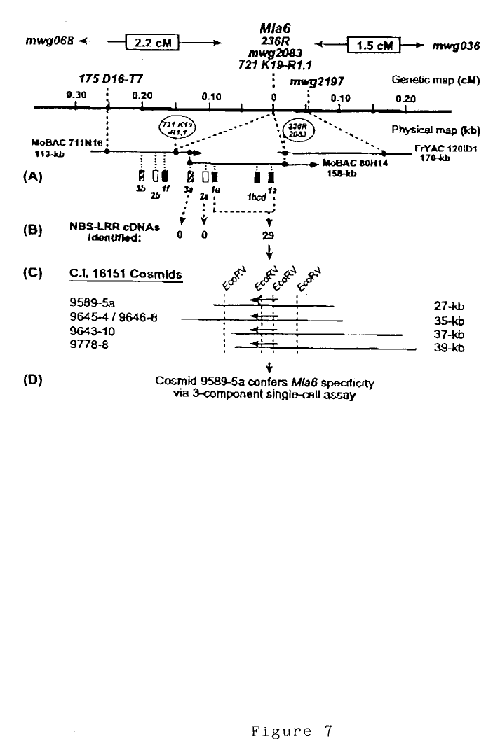

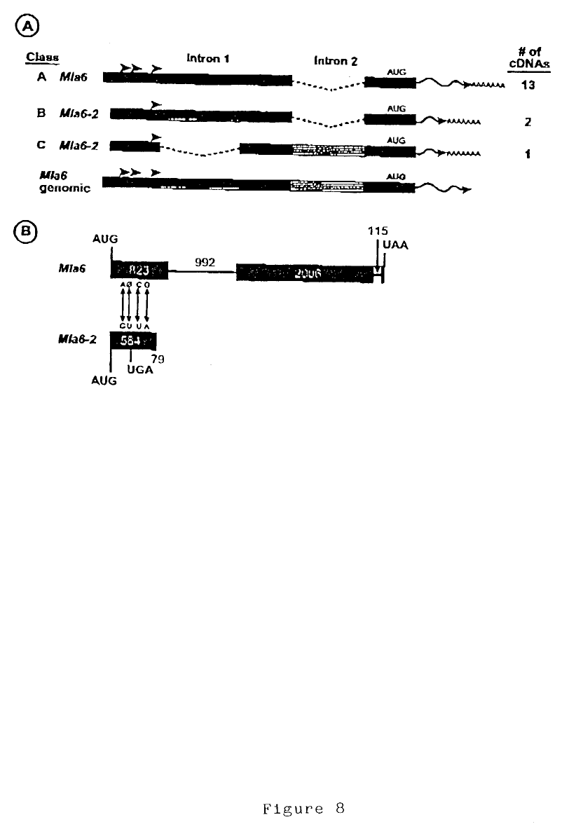

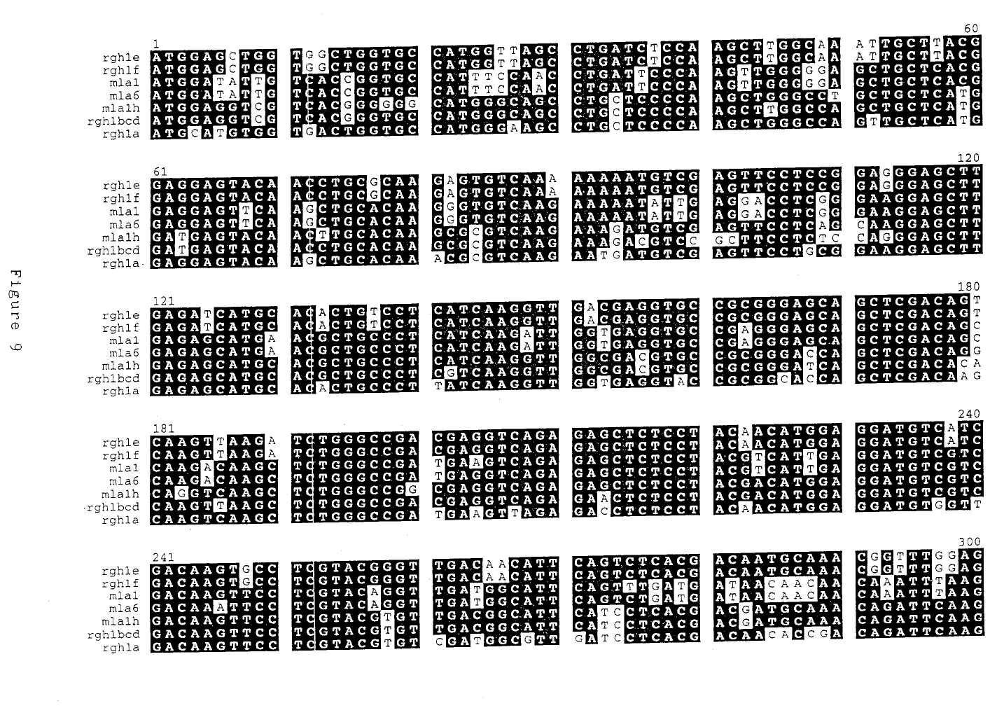

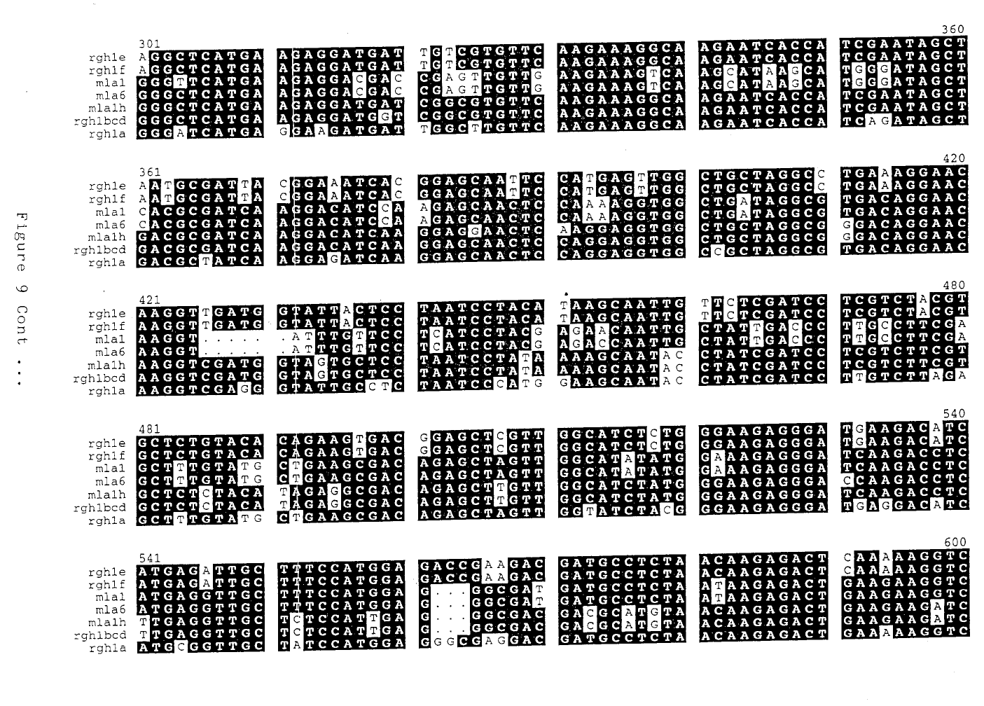

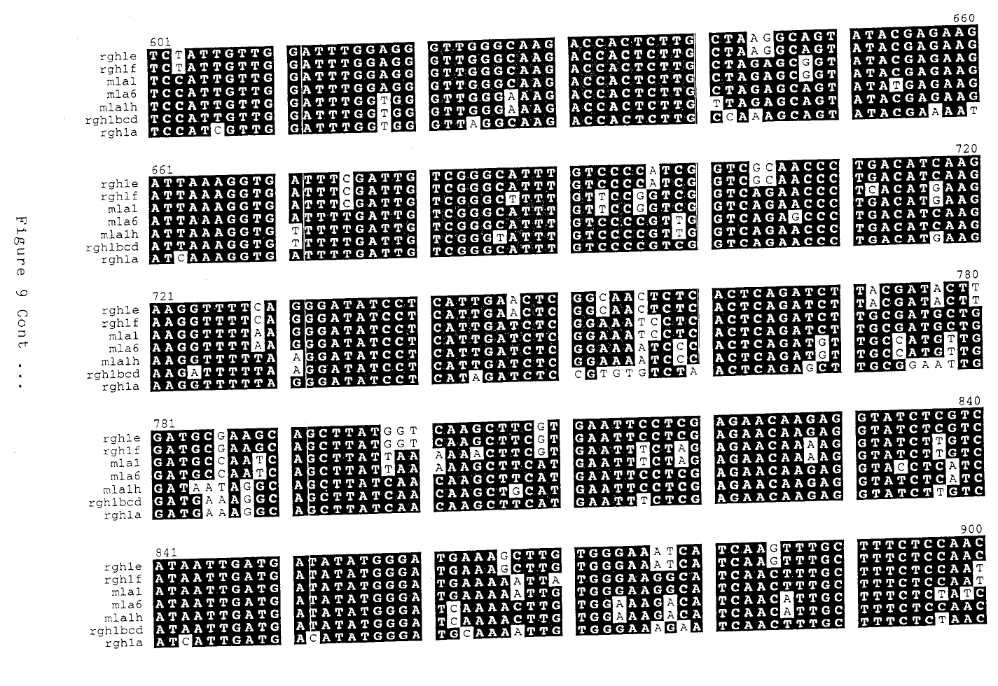

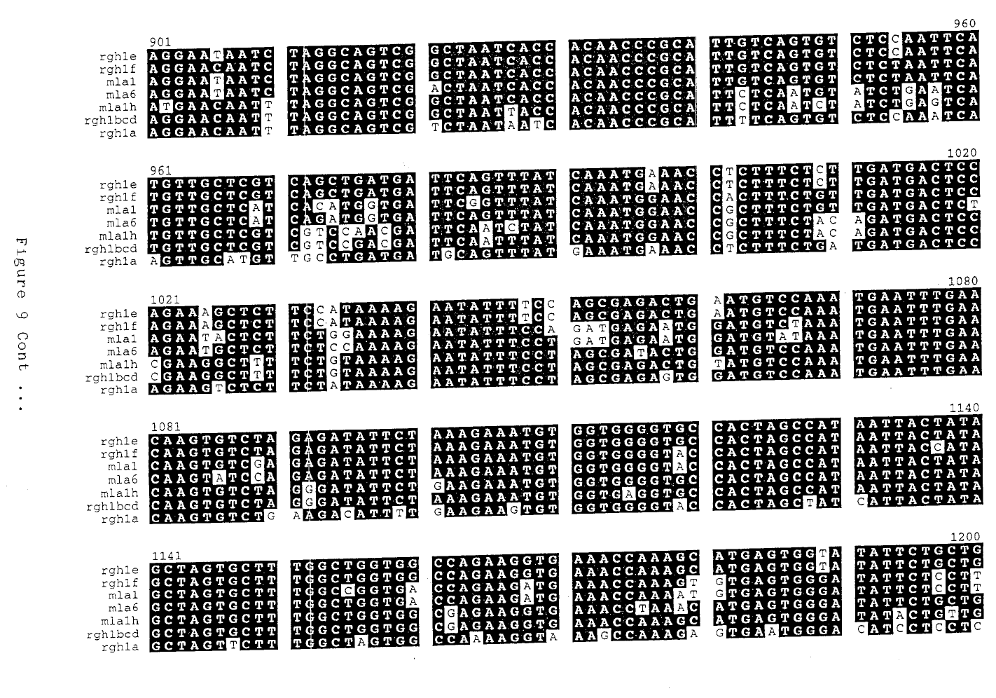

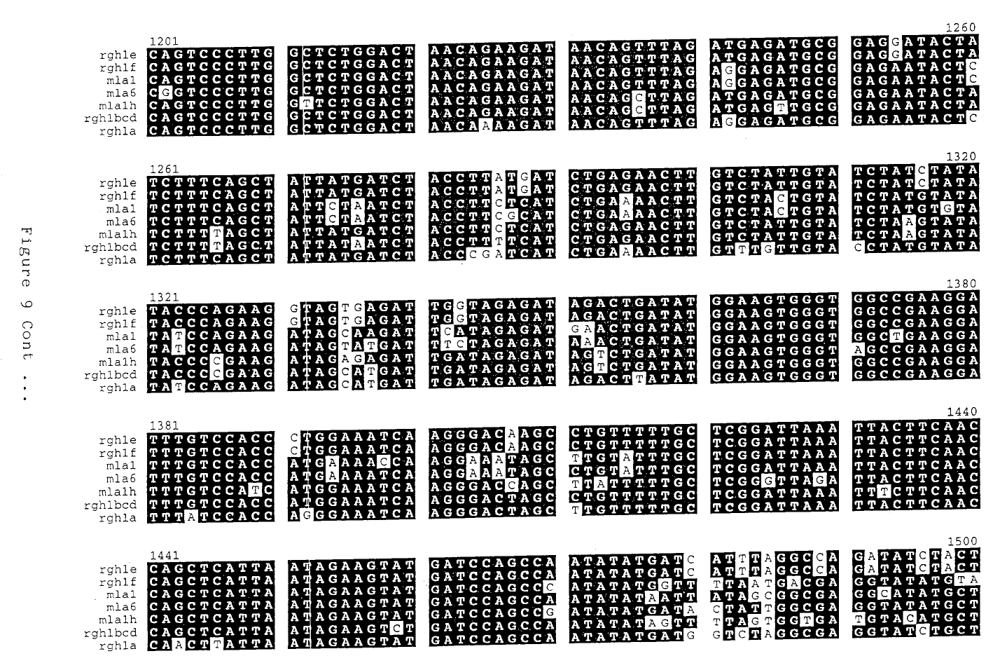

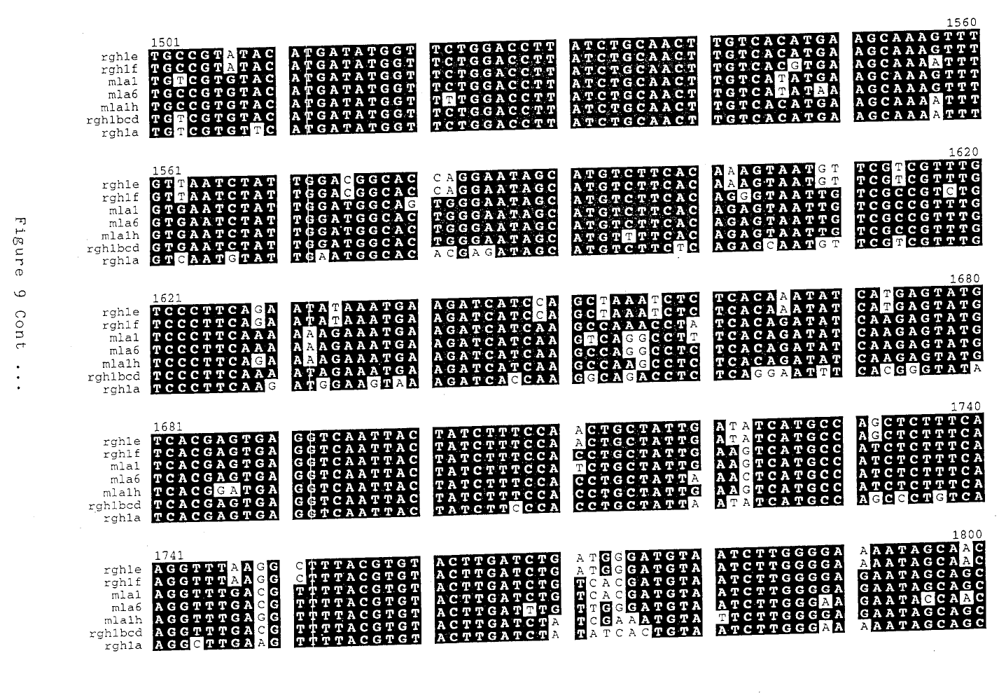

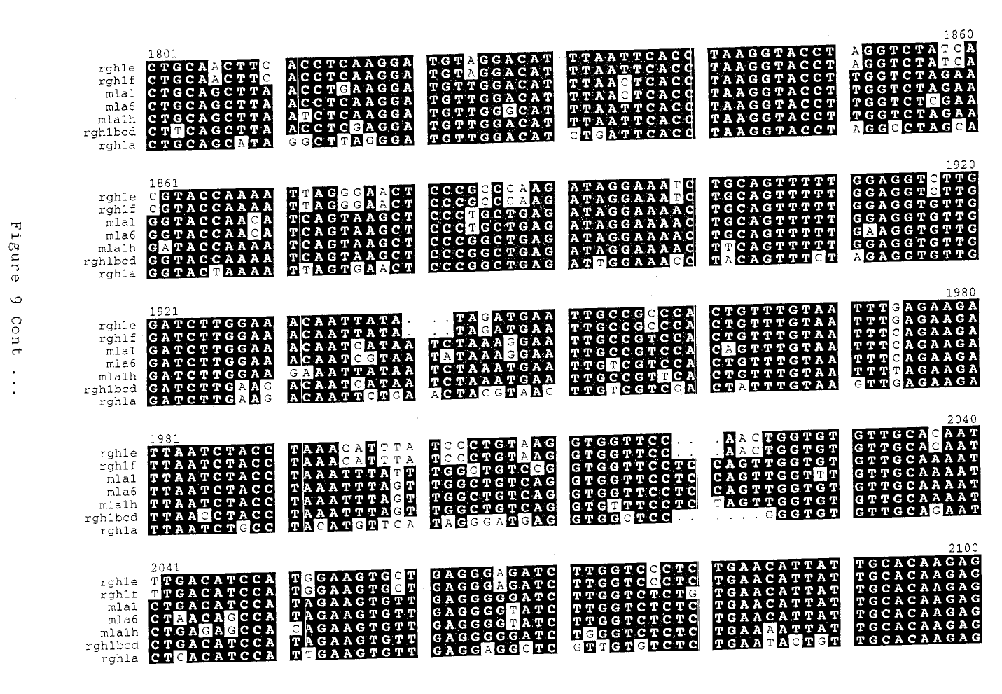

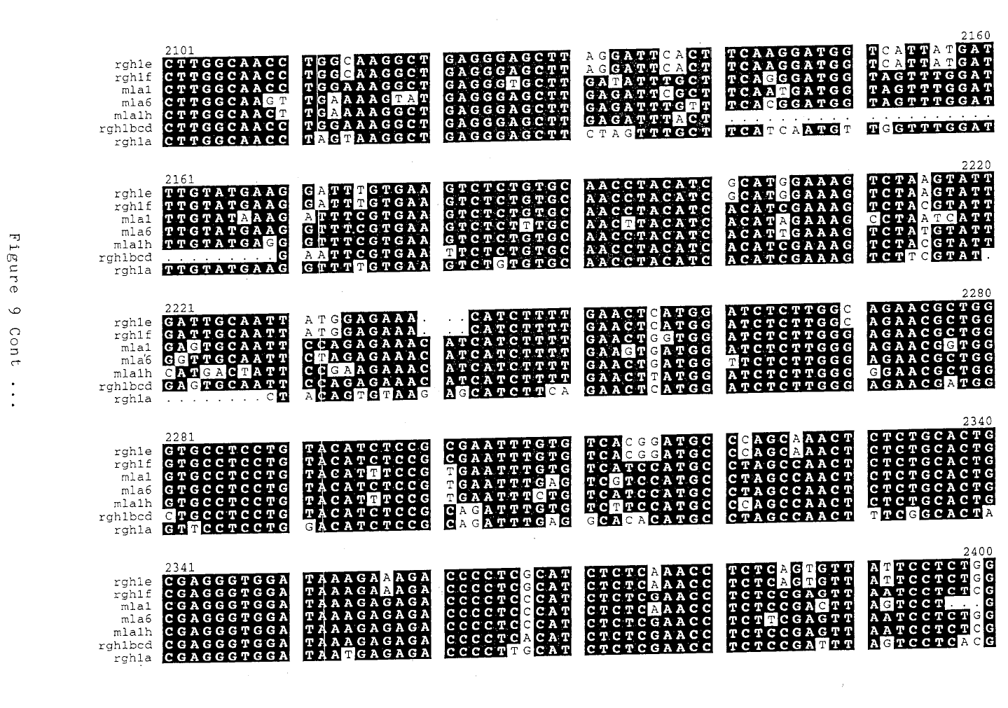

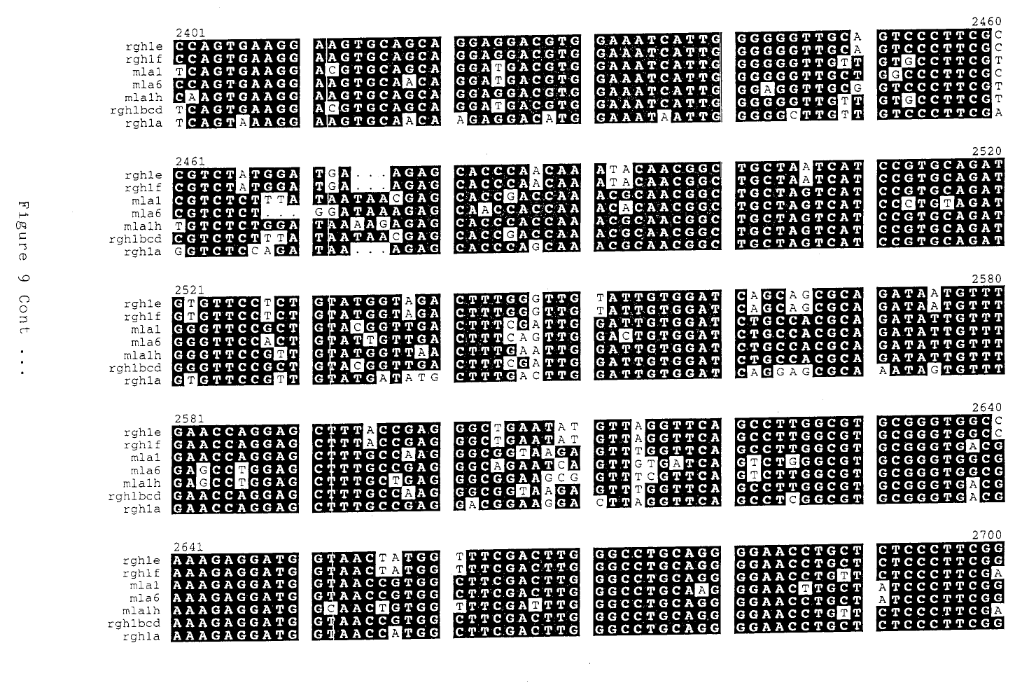

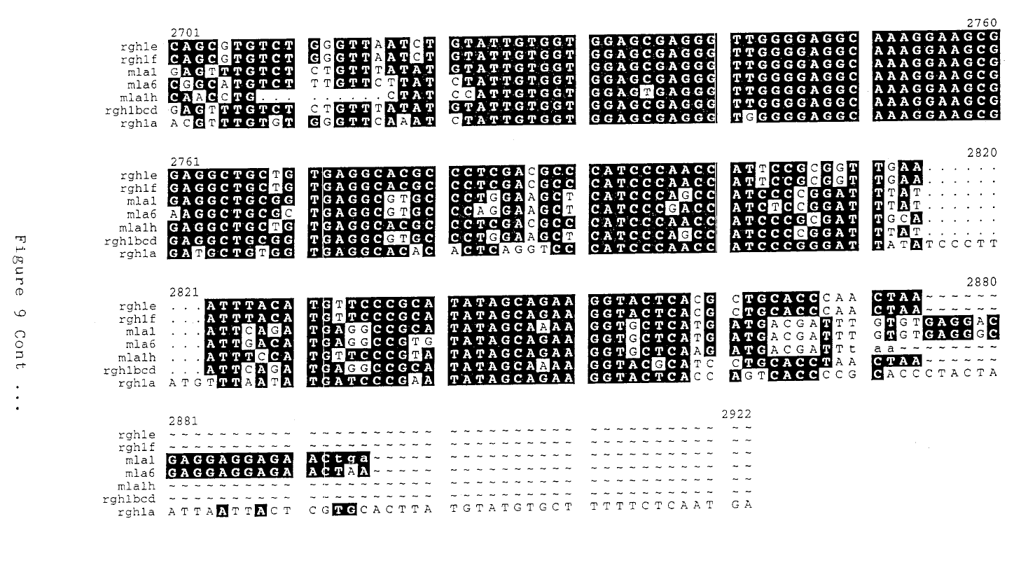

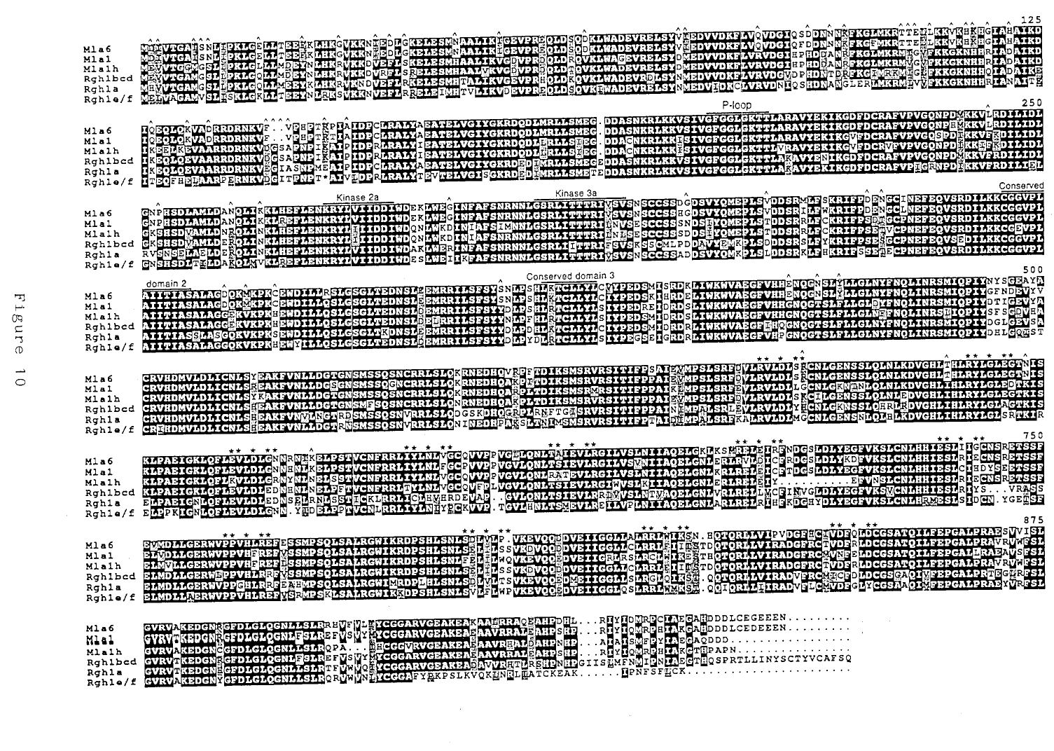

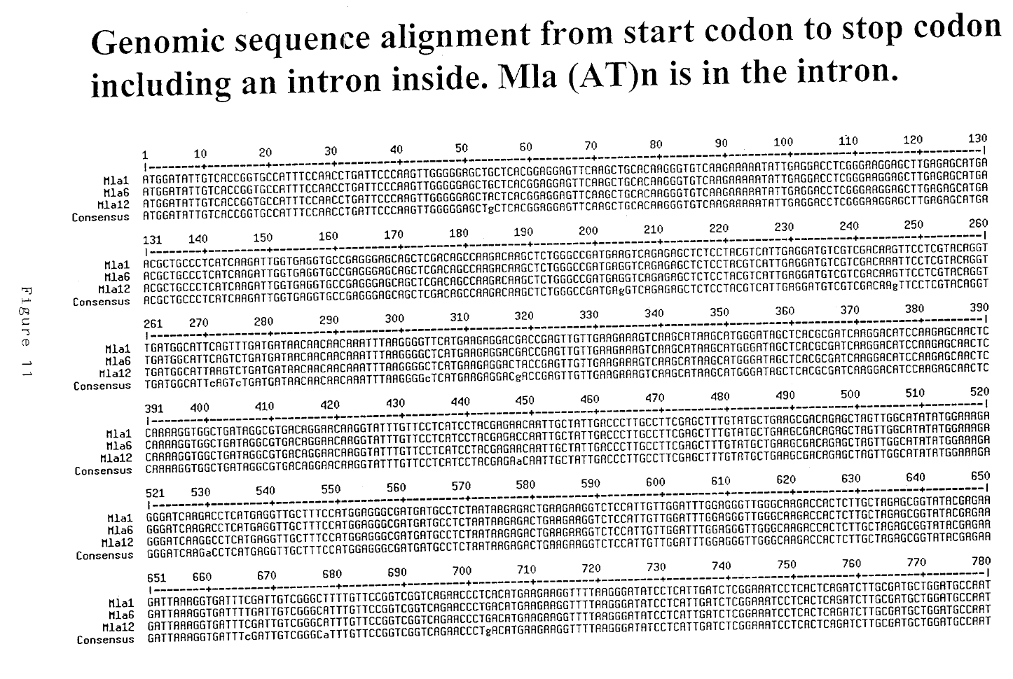

The potential presence of a gene in p6-49-2 mediating AvrMla1-dependent powdery mildew resistance motivated us to determine the DNA sequence of the cosmid clone. Towards this end, recently described standard protocols were employed (Shirasu et al., 1999 b). Three criteria were applied to search for genes in the genomic sequences: (i) homology to characterized genes or expressed sequence tags (ESTs) in the public databases; (ii) occurrence of extended high coding probabilities and (iii) application of a gene finder program (BCM gene finder). The analysis revealed only two genes in p6-49-2, both showing significant sequence-relatedness to NBS-LRR type R genes. These two genes were provisionally designated R gene A and B (FIGS. 3 and 4). Both genes revealed uninterrupted open reading frames, enabling us to deduce conceptual protein sequences of 958 and 949 amino acids, respectively (FIGS. 5 and 6). Interestingly, genes A and B are highly sequence-related to each other (82% DNA sequence identity and 78% identity at the amino acid level), suggesting that they might have arisen by a recent gene duplication event.

To test the function of R genes A and B separately by transient expression in detached leaves, a 15 kb EcoRI subclone containing only R gene A was isolated and designated p6-49-2-15. Similarly, a 7 kb DraI subclone containing only R gene B was isolated and designated p6-49-2-7. Representative results from co-bombardment experiments of the cosmid subclones together with pUGLUM are listed in Table 3. Interestingly, AvrMla1-specific powdery mildew resistance was only detected in transfected cells containing p6-49-2-15 whereas transfection of p6-49-2-7 supported similar high numbers of powdery mildew colonies upon challenge with isolates A6 or k1. These data strongly suggest that R gene A is Mla1 whereas the closely sequence-related R gene B is a non-functional RGH, i.e. it does not recognize any avirulence gene present in fungal isolates k1 and A6.

PCR primers specific for Mla1 were then used to amplify gene stretches from another randomly selected Mla1 mutant, M598, for direct DNA sequencing. A single nucleotide substitution (A to T) was identified in M598 in comparison to the Mla1 ‘wild-type’ sequence. This mutation changes the nucleotide triplet encoding Arg193 to a stop codon, thereby leading at the amino acid level to a truncated protein lacking 80% of the wild type protein sequence. Consistent with this observation, mutant M598 exhibits a fully susceptible infection phenotype.

### Example 7

### Isolation of Putative Mla6 From C.I. 16151

### Materials and Methods

Sequence data from BAC 80H14 was utilized to design of a series of PCR primers in an attempt to amplify homologous regions from genomic DNA of C.T. 16151 (Mla6). Low-copy number probes were designed from the LRR regions of the three RGH families. Erysiphe graminis f. sp. hordei isolates A6 (virMla1, AvrMla6) and k1 (AvrMla1, virMla6) were propagated on H. vulgare cv. Golden Promise and Ingrid, respectively, at 22° C. (16 h light/8 h darkness). A cDNA library was constructed with the assistance of D-W Choi, T. J. Close lab (UC Riverside) using the Uni-ZAP XR Library kit (Stratagene). The library was constructed from mRNA isolated from both uninoculated barley seedlings and seedlings inoculated with E. graminis f. sp. hordei isolate 5874 (AvrMla6) Tissue was harvested at both 20 and 24 hours post inoculation and snap-frozen in liquid nitrogen. The cDNA library was screened using probes derived from the LRR region of previously described resistance gene homologues RGH1a, RGH1e, RGH2a, and RGH3a (see Table 4).

RGH1a and RGH1e represent the Mla-RGH1 family where all members of this family have greater than 81% nucleic acid similarity. RGH2a and RGH3a are each 100% similar to other members of their respective families due to a large duplication in the Mla region of the barley genome.

DNA sequencing and oligonucleotide synthesis was performed by the Iowa State University DNA Sequencing and Synthesis facility.

Cosmid library construction was done in cooperation with Cell & Molecular Technologies, Inc. (Phillipsburg, N.J.). High-molecular weight genomic DNA from C.I. 16151 was partially digested with Sau3A, size selected for fragments ranging between 50 and 75 kb, and ligated into the BamHI site of digested cosmid SuperCos-1 (Stratagene, La Jolla, Calif.). Ligated cosmids were then electroporated into the XL-1 Blue strain of E. coli. The library was amplified in semi-solid medium and aliquoted into 347 pools containing between 7,500 and 10,000 clones each. An aliquot (0.5 μl; ˜5×10⁶ clones) of each bacterial pool was placed in a PCR reaction with Mla6 cDNA primers (see Table 4). Pools from which the appropriately sized PCR product could be amplified were diluted and plated onto solid media. Individual cosmids were identified by colony hybridization using the Mla6 cDNA as a probe. A plasmid library of partially digested 9589-5a DNA was constructed in pCGEM-7Zf(+) (Promega, Madison, Wis.) and 384 templates were sequenced.

### Screening Results

Previous research resulted in the development of a physical contig of YAC and BAC clones cosegregating with and spanning the Mla locus. Sequence analysis of Mla-spanning BACs from cv. Morex revealed the presence of three families of NBS-LRR resistance gene homologues (RGHs). These families were designated RGH1, RGH2, and RGH3 based on their sequence divergence (Wei et al., 1999). Although Morex does not contain a characterized Mla resistance specificity, we utilized the information derived from our physical mapping efforts to identify candidates for the Mla6 allele present in C. I. (Cereal Introduction) 16151, a Franger-derived, near-isogenic line (Moseman, 1972). Genomic DNA of C.I. 16151 was used as substrate for PCR amplification of the LRR regions from the three Mla-RGH families.

The 39F13 and 39B95 primers amplified sequences corresponding to the LRR of Mla-RGH1a , 38F19 and 38B27 amplified sequences corresponding to the LRR of Mla-RGH1e, 38IF50 and 38IB62 amplified sequences corresponding to the LRR of Mla-RGH2a, and 80H14R1F30 and 80H14R1B35 amplified sequences corresponding to the LRR of Mla-RGH3a (Table 4). The resulting amplified DNAs were used to screen 400,000 pfu of a Lambda-Zap cDNA library constructed from C.I. 16151 (Mla6) seedlings inoculated with an avirulent isolate of powdery mildew. No confirmed plaques hybridized to the Mla-RGH2a or Mla-RGH3a probes, however, 62 cDNAs hybridized to the mixed Mla-RGH1a/RGH1e probe(Table 4; FIG. 7). These low-copy genomic DNAs were used individually to hybridize to 400,000 pfu of an unamplified Lambda-Zap cDNA library constructed from C.I. 16151 seedlings inoculated with an AvrMla6-containing isolate of Bgh (see Methods). No plaques were identified using the Mla-RGH2a or Mla-RGH3a probes, however, 29 cDNAs with homology to the NBS-LRR class of plant disease resistance genes hybridized to a mixed Mla-RGH1a/RGH1e probe. Thirteen of the 29 cDNAs contained 5′ untranslated regions (UTRs) up to 400-nt in length. The largest of the cDNAs was used as a probe to re-screen the same library, which resulted in the isolation of 9 previously unidentified cDNAs, including 2 truncated classes with no NBS- or LRR-encoding domain. In total, this screen revealed the presence of three classes of transcripts with 5′ UTRs, containing 13, 2, and 1 members, respectively.

### Architecture of Mla-RGH1 cDNAs

As shown in FIG. 8, members of cDNA classes B and C are severely truncated and contain only 663 nucleotides (nt) after the start AUG, compared to the 2871-nt open reading frame of class A. The first 584-nt of the ORFs contain 4 nucleotide differences between class A and classes B and C. One of these mutations, an insertion at base 250 in the open reading frame of classes B and C, causes a frame shift leading to termination of the protein sequence after only 87 amino acids. Another striking difference between these classes occurs 584-nt downstream of the start AUG, where 79 nt of classes B and C have no significant similarity to class A cDNAs.

Significant differences between the 3 classes of RGH1 cDNAs were also found within the 5′ UTRs. Aside from different intron splicing events, the 5′ UTRs of classes B and C contain identical nucleotide sequences, but are different from class A cDNAs in a small region near the 5′ end (see FIG. 8). This divergent region in the first cDNA class is 68-nt in length and contains two 17-nt repeated sequences separated by 10 bases. In contrast, in classes B and C, this region is 28-nt shorter and is identical to the corresponding section of the 5′ UTR of Mla1 (Zhou et al., in press) but shares no similarity to class A cDNAs. In summary, these data suggest the presence of separate genes encoding class A and class B/C cDNAs. The presence of at least two genes is corroborated by the observation of 3 or more hybridizing restriction fragments with multiple enzymes on genomic DNA gel blots (data not shown). The fact that class B and C cDNAs were isolated implies that the gene encoding them contains a functional promoter, although premature termination within the open reading frame and the absence of any NBS or LRR encoding sequence suggests that the function of these proteins could he compromised. Therefore, we focused on determining whether the gene encoding class A alone is capable of conferring Mla6 specificity.

To confirm that the candidate Mla6 cDNA was indeed a functional copy encoding the Mla6 specificity, we isolated a genomic copy (including the upstream native promoter—see Annexes below) for use in the single-cell transient assay for powdery mildew resistance. To identify a genomic clone of the Mla6 gene, we screened a three-genome-equivalent cosmid library constructed from genomic DNA of the identical near-isogenic line (C.I. 16151) that was used to construct the cDNA library. As illustrated in FIG. 7, 347 pools containing 3.42×10⁶ cosmid clones (˜10,000 clones/pool) were screened via PCR utilizing several primer pairs derived from the Mla6-candidate cDNA. Seven pools yielded PCR products that were the same size as products amplified from C.I. 16151 genomic DNA. Individual cosmids that were purified from these pools ranged between 27- and 38.7-kb in length. DNA gel-blot analysis of EcoRI, HindIII, EcoRV, and BclI digested cosmids and subsequent hybridization with the RGH1 class A cDNA probe revealed that 5 cosmids contained identical restriction site patterns as found in the class A cDNA sequence (FIG. 7), whereas the other two cosmids contained related, but not identical, cross-hybridizing members.

Cosmid 9589-5a was sequenced (see Annexes below). Sequence analysis identified a putative open reading frame identical to the first class of Mla6 cDNAs. The 5′ UTR contained within the cosmid sequence is also identical to the class A cDNAs and shows the presence of the 2 putative introns. Only the second intron is spliced out of the UTR of the Mla6-candidate cDNA.

### Example 8

### Functional Complementation of the Mla6 Specificity in 3-component Transient Assay

### Methods

Biolistic bombardment of leaves was carried out generally as described above. Detached leaves of seven day old barley or wheat seedlings were placed onto 1% PHYTAGAR (Gibco) plates supplemented with 3% sucrose and allowed to recover for 1 hour at room temperature. Gold particles (BioRad) were coated with plasmid and/or cosmid DNA, accelerated with 7 bar (barley) or 9 bar (wheat) He gas into air of 100 mbar and delivered to the leaves. The leaves were then incubated at room temperature for 4 hours and transferred to 1% PHYTAGAR prior to fungal inoculation. The inoculated leaves were incubated at 15° C. (16 h light/8 h darkness) for 5 days (barley) or 1.5 days (wheat).

Barley cells expressing GFP were visualized 5 days after fungal inoculation using a microscope with an excitation filter of 480/40 nm, a dichromatic mirror at 505 nm and a green barrier filter of 510 nm.

Wheat leaves were vacuum-infiltrated twice with a GUS staining solution containing X-gluc and incubated at 37° C. overnight. The leaves were rinsed briefly with water and then immersed in Coomassie blue stain (50% methanol, 0.05% Coomassie brilliant blue R-250, 10% acetic acid, 40% water) for 15 minutes and rinsed again before visualization using a light microscope.

### Results

Seven-day old mlo-5 barley seedlings were bombarded with the GFP-Mlo reporter plasmid (pUGLUM) alone, pUGLUM and 9589-5a DNA, or pUGLUM and a cosmid containing Mla1 as a control. The leaves were given a short recovery period on water agar to allow GFP and Mlo expression. GFP fluorescing cells are rendered susceptible to E. graminis, due to the presence of wild-type Mlo.

Resistance specificities conferred by Mla6 (and Mla1) are the earliest and most effective at reducing fungal infection of the various Mla alleles (Wise and Ellingboe, 1983). The assay for Mla6 specificity was as follows: One set of leaves was inoculated at high density with E. graminis isolate A6, which contains AvrMla6 but not AvrMla1, and therefore is avirulent on cells with a functional Mla6 but virulent on cells that contain Mla1. As an inoculation control, a duplicate set of leaves was inoculated with E. graminis isolate k1, which does not possess AvrMla6 but contains AvrMla1. Seven days post-inoculation, GFP-Mlo expressing cells were scored. Only GFP fluorescing cells that had an attached fungal spore were counted in these experiments. Fluorescent cells that supported growth of a fungal colony were considered susceptible. The GFP cells that showed no fungal growth but had an attached spore were considered resistant. If the candidate RGH encodes Mla6 specificity, there will be significantly fewer conidiophores (sporulating structures for E. graminis) on the GFP-Mlo expressing cells inoculated with A6 than with k1.

The results of the above-described experiments are presented in Table 5.

It should be noted that the Mla6-containing line, C.I. 16151, is known to possess an additional Mla resistance specificity, designated Mla14 (Jørgensen, 1994). While Mla6 confers rapid and complete resistance to Bgh, Mla14 is expressed much later and only moderately suppresses sporulation of the fungus. Since Mla6 is epistatic to Mla14 and the two specificities cosegregate in coupling (Wei et al., 1999), Mla14 can only be detected if the infecting Bgh isolate possesses AvrMla14, but lacks AvrMlaG. The powdery mildew isolate that we have used does indeed contain AvrMla6 and, hence, the results described below focus on the complementation of Mla6 specificity.

In leaves that were bombarded with pUGLUM DNA alone, there was no difference in susceptibility after inoculation with the A6 or K1 conidia. Growth of isolates A6 and k1 was observed in 50.0% and 52.3% of GFP cells, respectively. Results of previous experiments using this system suggest that fungal growth in 45% to 60% of GFP cells should be considered complete susceptibility. When cosmid 9589-5a DNA was included in the bombardment, the percentage of GFP cells that support growth of isolate A6 was reduced to 9.4%. Cells inoculated with k1 conidia supported fungal growth 46.5% of the time, which is not significantly different from that of the control. In the reverse experiment, a cosmid containing Mla1 reduced susceptibility to the AvrMla1 containing, k1 isolate but did not affect susceptibility to A6. These data clearly indicate that the gene encoded within cosmid 9589-5a is capable of conferring resistance to E. graminis isolate A6 expressing AvrMla6.

Thus, because this functional Mla6 sequence is identical to the proposed Mla6 cDNA and it co-segregates with the Mla6 phenotype in our high-resolution mapping population, we consider this gene to be the functional copy of the Mla6 allele.

### Example 9

### The Structure of Mla6

The deduced protein sequence of the Mla6 open reading frame contains 955 amino acids with an estimated molecular mass of 107.75 kDa. An in-frame stop codon 33-nt upstream of the putative start methionine confirms that the identified ORF is the entire coding region of Mla6. A COILS (v. 2.1; Lupas et al., 1991) analysis of the MLA6-protein sequence revealed with greater than 95% probability that a coiled-coil region is located between amino acids 24 and 50, suggesting that MLA6 belongs to the coiled-coil subset of NBS-LRR resistance proteins. Two potential myristoylation sites are also located at the N-terminus of the MLA6 protein sequence. These potential myristoylation sites, located at amino acids 6-11 and 28-33, suggest that post-translational modification may lead to localization of the protein to the plasma membrane. Another cytoplasmic resistance gene, Pto, also contains a potential myristoylation motif. However, site-directed mutagenesis of the invariant glycine residue has shown that myristoylation is not required for Pto-mediated resistance.

The MLA6 protein contains the 5 conserved motifs indicative of a nucleotide binding site (see FIG. 10). The kinase-1a (P-loop), kinase-2a, kinase-3a, and conserved domain 2 motifs are all highly conserved when compared to other NBS-LRR resistance proteins (Grant et al., 1995). However, the conserved NBS domain 3 of MLA6 lacks the conserved phenylalanine found in other NBS-containing resistance proteins. The C-terminal region of the protein contains 11 imperfect leucine-rich repeats with an average size of 26 amino acids. These LRRs conform to the consensus motif LxxLxxLxxLxLxx(N/C/T)x(x)L observed in other cytoplasmic R gene products (Jones and Jones, 1997).

### Example 10

### Comparison of Functional and Non-functional Mla Alleles

To deduce the conserved amino acids necessary for function of Mla alleles, the MLA6 protein sequence was compared to MLA1, an MLA1 homologue (MLA1-2) and four MLA-RGH1 family members from the barley cultivar Morex (FIG. 10).

Although there is a high level of conservation between all these sequences, it is apparent that the two functional proteins, MLA6 and MLA1, are much more similar to each other than to any of the non-functional proteins. MLA6 and MLA1 are 92.2% similar (91.2% identical) at the amino acid level. The MLA-RGH1 protein with the highest similarity to these two proteins is MLA-RGH1bcd, which is 87.3% similar (83.6% identical) to MLA1 and 84.2% similar (79.9% identical) to MLA6. Hence, there is only a 5-8% difference between the known functional and putative non-functional proteins. There are exactly 57 amino acids that are conserved between the ˜950aa

MLA6 and MLA1 proteins that are not shared with any of the non-functional alleles. The majority (38) of these differences are located within the first 160 amino acids.

A comparison of the leucine-rich repeats of these proteins reveals a number of “islands” of non-conserved amino acids that appear to be centered mainly around the putative solvent exposed residues of the repeats. The predicted solvent exposed residues in LRR regions of many R gene products are known to be hypervariable. Amino-acid variations within these exposed residues are thought to determine recognition specificity (Jones and Jones 1997; Botella et al. 1998).

Our results indicate that residues within these regions are highly variable not only between functional and non-functional proteins but also between the two functional proteins, MLA6 and MLA1 (FIG. 10). Further analysis suggested that this variability may be under positive selection. In any given region of a gene, a greater number of non-synonymous (Ka) than synonymous (Ks) mutations indicates selective divergence of the region (Kₐ/Kₛ>1; Parniske et al., 1997; Hughes and Yeager, 1998; Meyers et al., 1998).

The ratio of non-conserved muations to conserved mutations between the solvent exposed residues of Mla6 and Mla1 is 3.75 (15/04) suggesting selection for divergence at these residues. Comparatively, the entire LRR region has a Kₐ/Kₛ ratio of 1.64 (36/22) and the region upstream of the LRR has a ratio of exactly 1.0 (26/26).

Therefore, there appear to be two regions of divergence between Mla-RGH1 family members. The first region is located at the N-terminus of the protein which contains a large number of residues conserved between MLA6 and MLA1 but divergent among the non-functional proteins. This division between functional and non-functional alleles is not present in other parts of the protein, suggesting that this region may influence overall functionality. Divergence within the second region, the leucine-rich repeats, occurs among all the alleles. Amino acids within the LRR and, more specifically, within the solvent exposed residues appear to be under selective pressure for divergence.

### Example 11

### Mla6 and RAR1

Previously, the function of Mla 6-mediated resistance was shown to be dependent on Ral1 (Jørgensen, 1996; Shirasu et al., 1999). This conclusion was made based on genetic data obtained from Mla12-susceptible barley mutants (Torp and Jorgensen, 1986; Jorgensen, 1988). Mla1, however, has been shown to function independently of Ral1. To conclusively demonstrate whether Mla6-mediated resistance is dependent on the presence of a functional Ral1 gene using the single-cell assay, we tested whether cosmid 9589-5a was capable of conferring resistance in a rar1 mutant background. The rar1 -2 mutant plant used in this experiment has been described previously (Freialdenhoven et al., 1994; Shirasu et al., 1999).

However, to utilize this mutant in the 3-component single cell assay, a double mutant (mlo-5/rar1-2), previously isolated in a screen for mutations in genes that are required for mlo-specified resistance (Freialdenhoven et al. 1996) was used. The mlo-5/rar1-2 mutant leaves were bombarded with cosmid 9589-5a (Mla6) or a cosmid containing Mla1 (p6-49-2-15) as a negative control and then infected with E. graminis isolate A6 containing AvrMla6. No fungal hyphae were observed growing on GFP cells of mlo-5 mutant leaves after co-bombardment with cosmid 9589-5a, confirming the presence of a functional Mla6 allele. After bombardment with the cosmid that contained Mla1, 44.1% of the GFP cells supported fungal growth, indicating complete susceptibility. In contrast, mlo-5/rar1-2 leaves showed no significant difference between the percentage of hyphal growth sites on GFP cells after bombardment with cosmid 9589-5a or the cosmid containing Mla1. GFP cells of the mlo-5/rar1 mutant leaves bombarded with cosmid 9589-5a supported growth of isolate A6 41% of the time. Similarly, GFP cells of mlo-5/rar1 leaves bombarded with the cosmid containing

Mla1 supported A6 growth 43.7% of the time. These results clearly indicate that Mla6-mediated resistance is dependent on the presence a functional Ral1 gene and that, although Mla6 and Mla1 are structurally quite similar, they appear to utilize separate signaling pathways.

### Example 12

### Use of Mla6 in a Heterologous System

Recent research on Pto, N and Cf-9 demonstrated that these 3 different classes of dicot resistance genes are all able function in a heterologous system (Thilmony et al., 1995; Whitham et al., 1996; Hammond-Kosack et al., 1998).

This indicates that downstream signaling components necessary for function of some R genes are conserved among closely related species. To test whether the Mla6 CC-NBS-LRR resistance gene is functional in another closely related monocot, we used a variation of the 3-component transient assay described above to test whether the Mla6-containing cosmid, 9589-5a, could confer specificity to E. graminis in wheat (see Example 8 above for general methods). In this system, a reporter plasmid with GUS under the control of a ubiquitin promoter (pUGUS)(Schweizer et al, 1999, MPMI 12: 647-654) is used in place of the GFP reporter construct (pUGLUM) so that fungal haustorium can be easily visualized. After inoculation with the appropriate conidia, the leaves are incubated on water agar for 60 hours to permit the growth of haustorium. After this time, the leaves are first stained for GUS activity and then placed in Coomassie blue to stain the attached spores. A light microscope was used to detect the presence or absence of haustorium within GUS stained cells with an attached spore.

We bombarded cosmid 9589-5a into wheat leaves from the cultivar CERCO followed by inoculation with the wheat powdery mildew isolates JIW2 and JIW48. Both of these isolates are normally virulent on CERCO wheat. Co-bombardment of cosmid 9589-5a with pUGUS did not significantly decrease the percentage of infected cells when compared to bombardment with PUGUS alone (data not shown), suggesting that either wheat does not contain the machinery necessary for proper function of Mla6 or that JIW2 and JIW48 do not contain a recognized AvrMla6 gene product.

We therefore repeated the experiment using the barley powdery mildew strain A6, which contains a functional AvrMla6, to inoculate the bombarded wheat leaves. Although A6 is not completely virulent on wheat, it has been observed that infecting spores are able to form haustorium ˜30% of the time. Conidia from E. graminis f. sp. hordei isolate k1 are not able to form haustorium at a significant level and are not suitable for use as a negative control. Hence, the virulent E. graminis f. sp. tritici isolate JIW48 was used instead.

We tested whether cosmid 9589-5a is able to prevent the formation of haustorium in wheat cells infected with E. graminis f. sp. hordei isolate A6, but not after inoculation with E. graminis f. sp. tritici isolate JIW48. Seven-day old seedlings of wheat variety CERCO were bombarded with the GUS reporter plasmid (pUGUS) and cosmid 9589-5a, or with pUGUS and the Mla1 containing cosmid, as a negative control. After bombardment, duplicate leaves were inoculated with the appropriate powdery mildew isolates. Wheat cells bombarded with cosmid 9589-5a were susceptible to JIW48 spores 30.4% of the time. This level of susceptibility was also seen after bombardment with the Mla1 cosmid, with 30.6% of the GUS staining cells containing haustorium. After inoculation with the A6 spores, wheat cells bombarded with the Mla1 cosmid were susceptible 23.9% of the time, while only 9.2% of the cells bombarded with cosmid 9589-5a contained haustorium. The significant reduction of susceptible GUS-stained cells after co-bombardment with cosmid 9589-5a indicates that Mla6 is able to function in wheat to confer specificity to E. graminis f. sp. hordei expressing AvrMla6. The results are shown in Table 5.

### Example 13

### A Micro-satellite Tag for Functional Mla Genes—Cloning of Mla12

The sequence alignment of Mla1 and Mla6 using the Multalin program (version 5.4.1, www.toulouse.inra.fr/multalin) revealed high sequence identity from start codon to stop codon (94% on nucleotide sequence level). The most polymorphic region was in intron 3. The polymorphisms result from a simple sequence repeat (AT)ₙ. There are 14 repeats in Mla1, but only 8 (or 10) in Mla6. These findings suggest that functional Mla genes have a characteristic (AT)n repeat of varying length in intron 3. Mla1 and Mla6 belong to a big family of NB-LRR genes. There are many Mla homologues in the barley genome and other organisms as well. Interestingly, the (AT)ₙ repeat appears to be absent in all sequence-related non-functional Mla homologues that are physically linked within the Mla complex (Wei et al., 1999). By nucleotide sequence searching (www.ncbi.nlm.nih.gov/blast.cgi), we did not find any other NB-LRR genes or homologues in GENEBANK containing the (AT)ₙ repeat. Thus, the (AT)ₙ repeat sequence may serve as a signature of functional Mla genes in the complex Mla locus.

### Methods and Materials

A cosmid library of about 5 barley-genome equivalents was constructed using DNA from cultivar Sultan-5 containing the powdery mildew resistance gene Mla12, following the same procedures as those for the Mla1 cosmid library construction (Zhou et al., 2000). The library was screened by hybridization using the Mla1-LRR region (an insert from a plasmid clone pB76, see Zhou et al., 2000) as a probe, and eight positive clones were obtained. Low-pass sequencing of the positive clones revealed that one of them (named sp14-4) contains a CC-NB-LRR gene with (AT)36, the same micro-satellite as in Mla1 and Mla6. The sequence alignment of Mla1, Mla6 and the candidate Mla12 (FIG. 11) revealed high homology among them, and the most polymorphic region is inside the micro-satellite.

A cDNA library was constructed using mRNA obtained from infected leaves of Sultan-5 and screened by hybridization with Mla1-LRR region as probe. 10 positive clones were obtained that share 100% sequence identity to the ORF of CC-NB-LRR gene in sp14-4. However, none of them are full length clones. An adapter primer, OK172 (5′-CAGCCTCTTGCTGAGTGGAGATG-3′), and a gene specific primer MlaNBAS1 (5′-TCTTGCCCAACCCTCCAAATCC-3′) were used to amplify the 5′ region of the cDNA. The PCR products were cloned into pGEM-T vector (Promega), and of the sequenced clones, 6 contain the 5′ region of the CC-NB-LRR gene. A full-length cDNA sequence was obtained by over-lapping the 5′ region PCR product and the longest cDNA clone obtained (Annex IV). The encoded polypeptide product is shown in Annex V.

Gene specific primers (Table 6) were designed according to the sequence of the Mla12 candidate gene and PCR products were amplified from mla12 mutants. The PCR products were purified using QIAQUICK PCR purification kit (Quiagen), and sequenced. Two-point mutations inside the LRR region of the Mla12 candidate gene were found in two mutants respectively, M22, and M66.

TABLE 1
Cosmids Screened with Probes

| RGH-1a | RAH-1b | B2 | MWG2083 | MWG2197 |
|---|---|---|---|---|
| p5-33-1 | p7-35-1 | p3-91 | p7-35-2 | p5-3-1 |
| p5-42-2 | | p4-42-1 | | p7-24-1 |
| p6-49-2 | | | | P4-25-4 |
| p7-36-2 | | | | P6-16-1 |

TABLE 2

| | | | pUGLUM | | pUGLUM | |
|---|---|---|---|---|---|---|
| | pUGLUM | | + | | + | |
| | (GFP/Mlo) | | p7-35-2 (48 kb) | | p6-49-2 (49 kb) | |
| | GFP | GFP | GFP | GFP | GFP | GFP |
| | + | + | + | + | + | + |
| | Spore | Colony | Spore | Colony | Spore | Colony |
| −AvrMla1 | 180 | 73 | 97 | 42 | 66 | 29 |
| +AvrMla1 | 239 | 104 | 85 | 31 | 24 | 5 |

TABLE 3

| | pUGLUM | | | | | | | |
|---|---|---|---|---|---|---|---|---|
| | (GFP/Mlo) | | pUGLUM | | pUGLUM | | pUGLUM | |
| | + | | + | | + | | + | |
| | p7-35-2 (46 | | p6-49- | | p6-49-2-15 (15 | | p6-49-2-7 (7 | |
| | kb) | | 2 (49kb) | | kb) | | kb) | |
| | GFP | GFP | GFP | GFP | GFP | GFP | GFP | GFP |
| | + | + | + | + | + | + | + | + |
| | Spore | Colony | Spore | Colony | Spore | Colony | Spore | Colony |
| −Avr | 85 | 44 | 77 | 35 | 77 | 32 | 148 | 67 |
| Mla1 | | | | | | | | |
| +Avr | 79 | 39 | 50 | 7 | 45 | 1 | 139 | 53 |
| Mla1 | | | | | | | | |

TABLE 4

| | | | Sequence | | |
|---|---|---|---|---|---|
| | | Fragment | designation | Region | |
| Primer | Primer sequences | size | & | of RGH | Annealing |
| designation | (5′->3′) | (bp) | origin | ORF | temperature |

| | | | | | |
|---|---|---|---|---|---|
| 39F13 | GGTTACCATCCTCTTTCGTCACC | 582 | RGH1a | LRR | 56 |
| 39B95 | GGAGGCTCGTTGTGTCTCTGAATAC | | (Morex) | | |
| 38F19 | TGGTTCCAACTGGTGTGTTGC | 426 | RGH1e | LRR | 54 |
| 38B27 | CCCCAATGATTTCCACGTCC | | (Morex) | | |
| 38IF50 | GCTCTCTCACTGTTCGTATGGACC | 198 | RGH2a | LRR | 54 |
| 38IB62 | AGCAGCTACCAGGCTGTATTGC | | (Morex) | | |
| 80H14BF | TGCTTTACCTCAAGTTGGCTGC | 212 | RGH3a | LRR | 56 |
| 30 | CGAAGGTGTGTGATTTCGATGC | | (Morex) | | |
| 80H14BB | | | | | |
| 9-1 | AAGCATGGGATAGCTCAC | 1433 | Mla6 | NBS-LRR | 58 |
| 53Rev3 | CCCAAGATTACATCGTGA | | CDNA | | |
| | | | (CI | | |
| | | | 16151) | | |
| 3UTRF | GCACGAGGTCATTCCAGAGATATG | 1616 | Mla6 | 5′UTR- | 58 |
| 53Rev4 | GAAAGAGAGTATTCTCCGC | | cDNA | NBS | |
| | | | (CI | | |
| | | | 16151) | | |

TABLE 5
(5 a)

| | mlo-5 leaves | | | |
|---|---|---|---|---|
| | A6 | | K1 | |
| | (AvrMla6/VirMla1) | | (VirMla6/AvrMla1) | |
| | spores | colonies | spores | colonies |
| pUGLUM (7 kb) | 52 | 26 | 44 | 23 |
| | | 50.0% | | 52.3% |
| pUGLUM/9589-5a | 127 | 12 | 129 | 60 |
| (27 kb) | | 9.4% | | 46.5% |
| pUGLUM/Mla1 (15 kb) | 51 | 24 | 54 | 5 |
| | | 47.1% | | 9.3% |

(5 b)

| | mlo-5 leaves | | mlo-5/rar1 leaves | |
|---|---|---|---|---|
| | A6 | | A6 | |
| | (AvrMla6/VirMla1) | | (AvrMla6/VirMla1) | |
| | spores | colonies | spores | colonies |
| pUGLUM/9589-5a | 72 | 0 | 78 | 32 |
| (27 kb) | | 0% | | 41.0% |
| pUGLUM/Mla1 (15 kb) | 102 | 45 | 87 | 38 |
| | | 44.1% | | 43.7% |

(5 c)

| | CERCO wheat leaves | | | |
|---|---|---|---|---|
| | A6 | | JIW48 | |
| | (AvrMla6/VirMla1) | | (VirMla6/VirMla1) | |
| | spores | haustorium | spores | haustorium |
| pUGUS/9589-5a | 119 | 11 | 92 | 28 |
| (27 kb) | | 9.2% | | 30.4% |
| pUGUS/Mla1 (15 kb) | 209 | 50 | 134 | 41 |
| | | 23.9% | | 30.6% |

TABLE 6

Gene-specific primers for PCR and sequencing of Mla12
from the mutants

| | | | Region |
|---|---|---|---|
| Primer | | Annealing | of |
| name | Primer sequence | temterature | Mla12 |
| Mla12- | 5′-CACCTCACCTTCTGTCTCTCTC | 55° C. | 1^{st} |
| S1a | | | exon |
| Mla12- | 5′-GCATCTTTCTTGCTATTCTGCTC | 55° C. | 1^{st} |
| S1b | | | intron |
| Mlal2- | 5′-TGCCATTTCCAACCTGATTCCC | 55° C. | 3^{rd} |
| S1c | | | exon |
| Mla12- | 5′-TCTCCCTCTTTCCTTCCTCTCC | 55° C. | 3^{rd} |
| AS1a | | | intron |
| Mla12- | 5′-CCTTTAATCTTCTCGTATACCGCTC | 55° C. | 3^{rd} |
| AS1b | | | exon |
| Mla12- | 5′-TGTTTAGTGTGAACTGCTTATGCC | 55° C. | 3^{rd} |
| AS1c | | | intron |
| Mla12- | 5′-CCTTGTTCCTGTCACGCCTATC | 55° C. | 3^{rd} |
| AS1d | | | exon |
| Mla12- | 5′-GATGCTTAATGAGAGTAAGATTATCGAG | 55° C. | 3^{rd} |
| S2a | | | intron |
| Mla12- | 5′-GAAGGGACAAACGACGACAATTACT | 55° C. | 4^{th} |
| AS2a | | | exon |
| Mla12- | 5′-GGCATCAACTTTGCTTTCTCCAATAG | 55° C. | 4^{th} |
| S2b | | | exon |
| Mla12- | 5′-CGACGACAATTACTCTGTGAAGAC | 55° C. | 4^{th} |
| As2b | | | exon |
| Mla12- | 5′-TAACAGTTTAGAGGAGATGCGG | 55° C. | 4^{th} |
| S3a | | | exon |
| Mla12- | 5′-ATGGAGAAAGGAAGGTAGGTGG | 55° C. | 4^{th} |
| AS3a | | | exon |
| Mla12- | 5′-TTAGAGGAGATGCGGAGAATAC | 55° C. | 4^{th} |
| S3b1 | | | exon |
| Mla12- | 5′-CTCCCGACTGAGATAGGAAAAC | 55° C. | 4^{th} |
| AS3b2 | | | exon |
| Mla12- | 5′-CACAATAGAGAAGAACAAAGACATC | 55° C. | 4^{th} |
| As3b | | | exon |
| Mla12- | 5′-TTGTTGTCCCTTCGTCGTCTCTGG | 55° C. | 4^{th} |
| S3c | | | exon |
| Mla12- | 5′-TGTGCGCCAAAAATCAGTTCTCAC | 55° C. | 5^{th} |
| AS3c | | | exon |

### Reference

These and the other references cited herein, inasmuch as they may be required to supplement the common general knowledge to support the present disclosure, are specifically incorporated herein by reference:

Aarts et al., (1998) Proc. Natl. Acad. Sci. USA 95, 10306-10311.

Baker et al., (1997) Science 276, 726-733.

Bennett and Smith (1991) Phil Trans R Soc London (Biol) 334: 309-345.

Bent (1996). Plant disease resistance: function meets structure.

Plant Cell 8, 1757-1771.

Botella et al., (1998) Plant Cell 10, 1847-1860.

Buschges et al., (1997) Cell 88, 695-705.

Collins et al., (1999). Plant Cell 11, 1365-1376.

DeScenzo et al., (1994) Mol. Plant-Microbe Interact. 7, 657-666.

Dixon et al.,(1998) Plant Cell 10, 1915-1925.

Ellingboe, A. H. (1972) Phytopathology 62, 401-406.

Ellis et al. (1999) Plant Cell 11, 495-506.

Fernandez et al., (1993) Biochim. Biophys. Acta. 1172, 346-348.

Feuillet (1999) Proc. Natl. Acad. Sci. U.S.A. 96, 8665-8670.

Flor, H. H. (1971) Annu. Rev. Phytopathol. 9, 275-296.

Freialdenhoven et al. (1994) Plant Cell 6, 983-994.

Grant et al., (1995) Science 269, 843-846.

Hammond-Kosack, K. E., and Jones, J. D. G. (1997) Annu. Rev. Plant Phys. Plant Mol. Biol. 48, 575-607.

Hammond-Kosack et al., (1998) Plant Cell, 10, 1251-1266.

Hu et al., (1996). Plant Cell 8, 1367-1376.

Huges, A. L., and Yeager, M. (1998). Annu. Rev. Genet. 32,415-435.

Iber, H. (1996). Biochim. Biophys. Acta. 1309, 167-173.

Jia et al. (2000) EMBO J. 19 (in press).

Jones, D. A., and Jones, J. D. G. (1997). Adv. Botan. Res. 24, 89-167.

Jørgensen, J. H. (1996). Genome 39, 492-498.

Jørgensen, J. H. (1994). Crit. Rev. Plant Sci. 13, 97-119.

Jørgensen, J. H. (1992). Plant Breed. 108, 53-59.

Jørgensen, J. H. (1988). Genome 30, 129-132.

Keen, N. T. (1990). Annu. Rev. Genet. 24, 447-463.

Keller, B., and Feuillet, C. (2000). Trends in Plant Sci. 5, 246-251.

Lupas et al., (1991) Science 252, 1162-1164.

Mahadevappa et al., (1994) Genome 37, 460-468.

McDowell et al., (2000) Plant J. 22, 523-529.

McDowell et al., (1998) Plant Cell 10, 1861-1874.

Mcintosh et al., (1998). Guidelines for nomenclature of biochemical/molecular loci in wheat and related species.

Proceedings of the 9th International Wheat Genetics Symposium. Volume 5, Catalogue of Gene Symbols for Wheat, pp. 2-11. Published by the University Extension Press, University of Saskatchewan, Canada.

Meyers et al., (1998) Plant Cell 10, 1833-1846.

Michelmore, R. W., and Meyers, B. C. (1998) Genome Res. 8, 1113-1130.

Moseman, J. G. (1972). Crop Sci. 12, 681-682

Nöel et al., (1999) Plant Cell 11, 2099-2111.

Parker et al., (1996) Plant Cell 8, 2033-2046.

Parniske et al. (1997). Cell 9, 821-832.

Peterhansel et al. (1997) Plant Cell 9, 1397-1409.

Richter et al., (1995) Genetics 141, 373-381.

Rommens et al., (1995) Plant Cell 7, 1573-1544.

Salmeron et al., (1996) Cell 86, 123-133.

Salmeron et al., (1994) Plant Cell 6, 511-520.

Schwarz et al (1999) Theor Appl Genet 98:521-530.

Scofield et al., (1996). Science 274, 2063-2065.

Shirasu et al., (1999a) Cell 99, 355-366.

Shirasu et al., (1999b) Plant J. 17, 293-299.

Shirley et al (1992) Plant Cell 4: 333-347.

Song et al., (1995) Science 270, 1804-1806.

Tang et al., (1996) Science 274, 2060-2063.

Thilmony et al. (1995). Plant Cell 7, 1529-1536.

Thomas et al. (1997) Plant Cell 9, 2209-2224.

Torp, J., and Jorgensen, J. H. (1986). Can. J. Genet. Cytol. 28, 725-731.

Van Der Bizen, E. A., and Jones, J. D. G. (1998). TIBS 23, 454-456.

Wei et al., (1999) Genetics 153, 1929-1948.

Whitham et al., (1996). Proc. Natl. Acad. Sci. 93, 8776-8781.

Whitham et al., (1994). Cell 78, 1101-1115.

Willis, A. E. (1999). Int. J. Biochem. Cell Biol. 31, 73-86.

Wise, R. P., and Ellingboe, A. H. (1983). Phytopathology 73, 1220-1222.

Sequences

Annex I - Mla6 ORF - FUNCTIONAL LENGTH: 2871

| | |
|---|---|
| 1 | ATGGATATTG TCACCGGTGC CATTTCCAAC CTGATTCCCA AGTTGGGGGA |
| 51 | GCTGCTCACG GAGGAGTTCA AGCTGCACAA GGGTGTCAAG AAAAATATTG |
| 101 | AGGACCTCGG GAAGGAGCTT GAGAGCATGA ACGCTGCCCT CATCAAGATT |
| 151 | GGTGAGGTGC CGAGGGAGCA GCTCGACAGC CAAGACAAGC TCTGGGCCGA |
| 201 | TGAGGTCAGA GAGCTCTCCT ACGTCATTGA GGATGTCGTC GACAAATTCC |
| 251 | TCGTACAGGT TGATGGCATT CAGTCTGATG ATAACAACAA CAAATTTAAG |
| 301 | GGGCTCATGA AGAGGAGGAC CGAGTTGTTG AAGAAAGTCA AGCATAAGCA |
| 351 | TGGGATAGCT CACGCGATCA AGGACATCCA AGAGCAACTC CAAAAGGTGG |
| 401 | CTGATAGGCG TGACAGGAAC AAGGTATTTG TTCCTCATCC TAGGAGACCA |
| 451 | ATTGCTATTG ACCCTTGCCT TCGAGCTTTG TATGCTGAAG CGACAGAGCT |
| 501 | AGTTGGCATA TATGGAAAGA GGGATCAAGA CCTCATGAGG TTGCTTTCCA |
| 551 | TGGAGGGCGA TGATGCCTCT AATAAGAGAC TGAAGAAGGT CTCCATTGTT |
| 601 | GGATTTGGAG GGTTGGGCAA GACCACTCTT GCTAGAGCGG TATACGAGAA |
| 651 | GATTAAAGGT GATTTTGATT GTCGGGCATT TGTTCCGGTC GGTCAGAACC |
| 701 | CTGACATGAA GAAGGTTTTA AGGGATATCC TCATTGATCT CGGAAATCCT |
| 751 | CACTCAGATC TTGCGATGCT GGATGCCAAT CAGCTTATTA AAAAGCTTCA |
| 801 | TGAATTTCTA GAGAACAAAA GGTATCTTGT CATAATTGAT GATATATGGG |
| 851 | ATGAAAAATT GTGGGAAGGC ATCAACTTTG CTTTCTCCAA TAGGAATAAT |
| 901 | CTAGGCAGTC GACTAATCAC CACAACCCGC ATTGTCAGTG TCTCTAATTC |
| 951 | ATGTTGCTCA TCAGATGGTG ATTCAGTTTA TCAAATGGAA CCGCTTTCTG |
| 1001 | TTGATGACTC TAGAATGCTC TTCTCCAAAA GAATATTTCC TGATGAGAAT |
| 1051 | GGATGTATAA ATGAATTTGA ACAAGTATCC AGAGATATTC TAAAGAAATG |
| 1101 | TGGTGGGGTA CCACTAGCCA TAATTACTAT AGCTAGTGCT TTGGCTGGTG |
| 1151 | ACCAGAAGAT GAAACCAAAA TGTGAGTGGG ATATTCTCCT TCGGTCCCTT |
| 1201 | GGCTCTGGAC TAACAGAAGA TAACAGTTTA GAGGAGATGC GGAGAATACT |
| 1251 | CTCTTTCAGC TATTCTAATC TACCTTCGCA TCTGAAAACT TGTCTACTGT |
| 1301 | ATCTATGTGT ATATCCAGAA GATAGTATGA TTTCTAGAGA TAAACTGATA |
| 1351 | TGGAAGTGGG TGGCTGAAGG ATTTGTCCAC CATGAAAATC AAGGAAATAG |
| 1401 | CCTGTATTTG CTCGGATTAA ATTACTTCAA CCAGCTCATT AATAGAAGTA |
| 1451 | TGATCCAGCC AATATATAAT TATAGCGGCG AGGCATATGC TTGCCGTGTA |
| 1501 | CATGATATGG TTCTGGACCT TATCTGCAAC TTGTCATATG AAGCAAAGTT |
| 1551 | TGTGAATCTA TTGGATGGCA CTGGGAATAG CATGTCTTCA CAGAGTAATT |
| 1601 | GTCGCCGTTT GTCCCTTCAA AAAAGAAATG AAGATCATCA AGTCAGGCCT |
| 1651 | TTCACAGATA TCAAGAGTAT GTCACGAGTG AGGTCAATTA CTATCTTTCC |
| 1701 | ATCTGCTATT GAAGTCATGC CATCTCTTTC AAGGTTTGAC GTTTTACGTG |
| 1751 | TACTTGATCT GTCACGATGT AATCTTGGGG AGAATAGCAG CCTGCAGCTT |
| 1001 | AACCTCAAGG ATGTTGGACA TTTAACTCAC CTAAGGTACC TTGGTCTAGA |
| 1851 | AGGTACCAAC ATCAGTAAGC TCCCTGCTGA GATAGGAAAA CTGCAGTTTT |
| 1901 | TGGAGGTGTT GGATCTTGGA AACAATCGTA ATATAAAGGA ATTGCCGTCC |
| 1951 | ACAGTTTGTA ATTTCAGAAG ATTAATCTAC CTAAATTTAG TTGGCTGTCA |
| 2001 | GGTGGTTCCT CCAGTTGGTT TGTTGCAAAA TCTAACAGCC ATAGAAGTGT |
| 2051 | TGAGGGGTAT CTTGGTCTCT CTGAACATTA TTGCACAAGA GCTTGGCAAG |
| 2101 | TTGAAAAGTA TGAGGGAGCT TGAGATTCGC TTCAATGATG GTAGTTTGGA |
| 2151 | TTTGTATGAA GGTTTCGTGA AGTCTCTTTG CAACTTACAT CACATAGAAA |
| 2201 | GCCTAATCAT TGGTTGCAAT TCTAGAGAAA CATCATCTTT TGAAGTGATG |
| 2251 | GATCTCTTGG GAGAACGGTG GGTGCCTCCT GTACATCTCC GTGAATTTGA |
| 2301 | GTCGTCCATG CCTAGCCAAC TCTCTGCACT GCGAGGGTGG ATAAAGAGAG |
| 2351 | ACCCCTCCCA TCTCTCAAAC CTCTCCGACT TAGTCCTGCC AGTGAAGGAA |
| 2401 | GTGCAACAGG ATGACGTGGA AATCATTGGG GGGTTGCTGG CCCTTCGCCG |
| 2451 | TCTCTGGATA AAGAGCAACC ACCAAACACA ACGGCTGCTA GTCATCCCTG |
| 2501 | TAGATGGGTT CCACTGTATT GTTGACTTTC AGTTGGACTG TGGATCTGCC |
| 2551 | ACGCAGATAT TGTTTGAGCC TGGAGCTTTG CCGAGGGCAG AATCAGTTGT |
| 2601 | GATCAGTCTG GGCGTGCGGG TGGCGAAAGA GGATGGTAAC CGTGGCTTCG |
| 2651 | ACTTGGGCCT GCAAGGGAAC TTGCTATCCC TTCGGCGGCA TGTCTTTGTT |
| 2701 | CTTATCTATT GTGGTGGAGC GAGGGTTGGG GAGGCAAAGG AAGCGAAGGC |
| 2751 | TGCGCTGAGG CGTGCCCAGG AAGCTCATCC CGACCATCTC CGGATTTATA |
| 2801 | TTGACATGAG GGCGTGTATA GCAGAAGGTG CTCATGATGA CGATTTGTGT |
| 2851 | GAGGGCGAGG AGGAGAACTA A |

Annex II - MLA6-A cDNA SEQUENCE
class1.seq (Mla6-A) Length: 3717
The following nucleotides are in the positions stated:
position 3303 (G), 3323 (G), 3328 (G), 3631 (C), 3644 (A),
and 3682 (C)

| | |
|---|---|
| 1 | GTCATTCCAG AGATATGCCA GTTGCGTTCT CACGGCTGAG TCATTGGCAC |
| 51 | CTCACCTTCT GTCTCTCTCG TTAAATTTGT ATCGATATAT AAGTGCTTTT |
| 101 | GAGTACTTGC ATATATAAGT GCTTTTGGAT CTAAAAAGTT ATTAGTTTTC |
| 151 | ATGCTTAAGT ATCTGATCAA TTTGCGGTGG TAGTGGCATC TTTCTTGCTA |
| 201 | TTCTGCTCTA ATGAAATCTT TCACGTCCAC ACGTTCTTGT TATAGATCTG |
| 251 | CTGATTTGCT TAGATTATAA GTTCTTCTTA TTCTTCCAGA TCGATTGGAG |
| 301 | CGACCCTCAC GCCTCTGGTG CGCCGTCGCT GTGTTCTGCT CCGCCGTGAA |
| 351 | GAATCAAGGC TTCCAGCTGA TTGATACGGA GATCTCGTCC TCCTGCTCTC |
| 401 | ATGGATATTG TCACCGGTGC CATTTCCAAC CTGATTCCCA AGTTGGGGGA |
| 451 | GCTGCTCACG GAGGAGTTCA AGCTGCACAA GGGTGTCAAG AAAAATATTG |
| 501 | AGGACCTCGG GAAGGAGCTT GAGAGCATGA ACGCTGCCCT CATCAAGATT |
| 551 | GGTGAGGTGC CGAGGGAGCA GCTCGACAGC CAAGACAAGC TCTGGGCCGA |
| 601 | TGAGGTCAGA GAGCTCTCCT ACGTCATTGA GGATGTCGTC GACAAATTCC |
| 651 | TCGTACAGGT TGATGGCATT CAGTCTGATG ATAACAACAA CAAATTTAAG |
| 701 | GGGCTCATGA AGAGGACGAC CGAGTTGTTG AAGAAAGTCA AGCATAAGCA |
| 751 | TGGGATAGCT CACGCGATCA AGGACATCCA AGAGCAACTC CAAAAGGTGG |
| 801 | CTGATAGGCG TGACAGGAAC AAGGTATTTG TTCCTCATCC TACGAGACCA |
| 851 | ATTGCTATTG ACCCTTGCCT TCGAGCTTTG TATGCTGAAG CGACAGAGCT |
| 901 | AGTTGGCATA TATGGAAAGA GGGATCAAGA CCTCATGAGG TTGCTTTCCA |
| 951 | TGGAGGGCGA TGATGCCTCT AATAAGAGAC TGAAGAAGGT CTCCATTGTT |
| 1001 | GCATTTGGAG GGTTGGGCAA GACCACTCTT GCTAGAGCGG TATACGAGAA |
| 1051 | GATTAAAGGT GATTTTGATT CTCGGGCATT TGTTCCGGTC GGTCAGAACC |
| 1101 | CTGACATGAA GAAGGTTTTA AGGGATATCC TCATTGATCT CGGAAATCCT |
| 1151 | CACTCAGATC TTGCGATGCT GGATGCCAAT CAGCTTATTA AAAAGCTTCA |
| 1201 | TGAATTTCTA GAGAACAAAA GGTATCTTGT CATAATTGAT GATATATGGG |
| 1251 | ATGAAAAATT GTGGGAAGGC ATCAACTTTG CTTTCTCCAA TAGGAATAAT |
| 1301 | CTAGGCAGTC GACTAATCAC CACAACCCGC ATTGTCAGTG TCTCTAATTC |
| 1351 | ATGTTGCTCA TCAGATGGTG ATTCAGTTTA TCAAATGGAA CCGCTTTCTG |
| 1401 | TTGATGACTC TAGAATGCTC TTCTCCAAAA GAATATTTCC TGATGAGAAT |
| 1451 | GGATGTATAA ATGAATTTGA ACAAGTATCC AGAGATATTC TAAAGAAATG |
| 1501 | TGGTGGGGTA CCACTAGCCA TAATTACTAT AGCTAGTGCT TTGGCTGGTG |
| 1551 | ACCAGAAGAT GAAACCAAAA TGTGAGTGGG ATATTCTCCT TCGGTCCCTT |
| 1601 | GGCTCTGGAC TAACAGAAGA TAACAGTTTA GAGGAGATGC GGAGAATACT |
| 1651 | CTCTTTCAGC TATTCTAATC TACCTTCGCA TCTGAAAACT TGTCTACTGT |
| 1701 | ATCTATGTGT ATATCCAGAA GATAGTATGA TTTCTAGAGA TAAACTGATA |
| 1751 | TGGAAGTGGG TGGCTGAAGG ATTTGTCCAC CATGAAAATC AAGGAAATAG |
| 1801 | CCTGTATTTG CTCGGATTAA ATTACTTCAA CCAGCTCATT AATAGAAGTA |
| 1851 | TGATCCAGCC AATATATAAT TATAGCGGCG AGGCATATGC TTGCCGTGTA |
| 1901 | CATGATATGG TTCTGGACCT TATCTGCAAC TTGTCATATG AAGCAAAGTT |
| 1951 | TGTGAATCTA TTGGATGGCA CTGGGAATAG CATGTCTTCA CAGAGTAATT |
| 2001 | GTCGCCGTTT GTCCCTTCAA AAAAGAAATG AAGATCATCA AGTCAGGCCT |
| 2051 | TTCACAGATA TCAAGAGTAT GTCACGAGTG AGGTCAATTA CTATCTTTCC |
| 2101 | ATCTGCTATT GAAGTCATGC CATCTCTTTC AAGGTTTGAC GTTTTACGTG |
| 2151 | TACTTGATCT GTCACGATGT AATCTTGGGG AGAATAGCAG CCTGCAGCTT |
| 2201 | AACCTCAAGG ATGTTGGACA TTTAACTCAC CTAAGGTACC TTGGTCTAGA |
| 2251 | AGGTACCAAC ATCAGTAAGC TCCCTGCTGA GATAGGAAAA CTGCAGTTTT |
| 2301 | TGGAGGTGTT GGATCTTGGA AACAATCGTA ATATAAAGGA ATTGCCGTCC |
| 2351 | ACAGTTTGTA ATTTCAGAAG ATTAATCTAC CTAAATTTAG TTGGCTGTCA |
| 2401 | GGTGGTTCCT CCAGTTGGTT TGTTGCAAAA TCTAACAGCC ATAGAAGTGT |
| 2451 | TGAGGGGTAT CTTGGTCTCT CTGAACATTA TTGCACAAGA GCTTGGCAAG |
| 2501 | TTGAAAAGTA TGAGGGAGCT TGAGATTCGC TTCAATGATG GTAGTTTGGA |
| 2551 | TTTGTATGAA GGTTTCGTGA AGTCTCTTTG CAACTTACAT CACATAGAAA |
| 2601 | GCCTAATCAT TGGTTGCAAT TCTAGAGAAA CATCATCTTT TGAAGTGATG |
| 2651 | GATCTCTTGG GAGAACGGTG GGTGCCTCCT GTACATCTCC GTGAATTTGA |
| 2701 | GTCGTCCATG CCTAGCCAAC TCTCTGCACT GCGAGGGTGG ATAAAGAGAG |
| 2751 | ACCCCTCCCA TCTCTCAAAC CTCTCCGACT TAGTCCTGCC AGTGAAGGAA |
| 2801 | GTGCAACAGG ATGACGTGGA AATCATTGGG GGGTTGCTGG CCCTTCGCCG |
| 2851 | TCTCTGGATA AAGAGCAACC ACCAAACACA ACGGCTGCTA GTCATCCCTG |
| 2901 | TAGATGGGTT CCACTGTATT GTTGACTTTC AGTTGGACTG TGGATCTGCC |
| 2951 | ACGCAGATAT TGTTTGAGCC TGGAGCTTTG CCGAGGGCAG AATCAGTTGT |
| 3001 | GATCAGTCTG GGCGTGCGGG TGGCGAAAGA GGATGGTAAC CGTGGCTTCG |
| 3051 | ACTTGGGCCT GCAAGGGAAC TTGCTATCCC TTCGGCGGCA TGTCTTTGTT |
| 3101 | CTTATCTATT GTGGTGGAGC GAGGGTTGGG GAGGCAAAGG AAGCGAAGGC |
| 3151 | TGCGCTGAGG CGTGCCCAGG AAGCTCATCC CGACCATCTC CGGATTTATA |
| 3201 | TTGACATGAG GCCGTGTATA GCACAAGGTG CTCATGATGA CGATTTGTGT |
| 3251 | GAGGGCGAGG AGGAGAACTA ATTTCTGATC CAGAGCGACT CACATTGCAT |
| 3301 | CANATGTGCT CTCGAGGTAG CANCGGCNCG GGGCGTTGGA GTTACAGCTG |
| 3351 | GTGGCATCAG AGATGCTTGT TTCACAAACA GTTCGGGCGG GCGCTGACCA |
| 3401 | TGCAAATGTT TCGAACTTTG CTGGAACTTG TGTGATGAGC TTCTTTTAAA |
| 3451 | TGGCACTCAG CTTGCAGAAA GAAACATGGT TTTGTTTTGT AATGAATAAG |
| 3501 | CAAGGGTGTT GGGGTGAATT GATCCTTACA AGGATAGCTT TGCTTTTCTT |
| 3551 | TAGTTGAGGG CCATCGTTGC TGCTCTGTTT TGCATGTTGT TGTTACATGG |
| 3601 | GAGGACATGC TAGTGTATTT TGTTTTTAAG NTGAGCCGAA CAANCCTGAG |
| 3651 | TATGTATTAT CAGTTCCGTG TTGAATGAAA TNTGAGCTCA TTAAAAAAAA |
| 3701 | AAAAAAAAAA AAAAAAA |

Annex IIIa - Mla6-A GENOMIC SEQUENCE
Mla6genomic1.seq Length: 6793
Mla6-A cDNA transcript in bold type

| | |
|---|---|
| 1 | AACTATGTTT AAAAAACTTC CAGGAATTTT TTGACTTTTT TTTAATTTCT |
| 51 | AAATTATTTT TAAATTCAGG TGCACTGGAA CATGAGACTC ATTGGGTATT |
| 101 | TCCGGTGTTG ATTTGAGGAG TAATTTACCA CCTGGCAAAT GACTGCATAG |
| 151 | ACAGAGGAGT AATGCATGAT GTGGACTGAC CAACCAACTG AGGAGATTCA |
| 201 | GAGAAATGAG AGGAGAGTAA ATGCAGTGAA TGATGGCTGG TGGACGGACC |
| 251 | ATATACAGTG TATGTAATTA TTTTGCTCTG AATCCCTGTC TCTCTGTGAC |
| 301 | CCACTGAATA AACACATCAG CCAAAAGCAG TACTGTTCGG ACTTCGGAGG |
| 351 | GATCGTGGAG TAGTAGTAAT TTCCTCTCTT GACTGTTGTT CCTCTGAGTC |
| 401 | CTGTGCTCCC CGCCTCCACT GACTGCTACC TCCATCTCGT CTCAGTCCTC |
| 451 | TCCTTCATTT CAAGCTGTGA ACCGAAAACA TGCACCCAGT CCGGCCTTGA |
| 501 | TGTAATGCAG GCAACCAATC GACATGGAGA TGTCGATTTT TAGCGTATAT |
| 551 | ATGCTTAGCC AGACCCAACT AGATCAAATA TGCAAGGTAC CTGAAAACGA |
| 601 | TGCCGGTAAC CCCAAATCGC GTCGTGAACC GGAGTAATGC TAGACTTACG |
| 651 | TAAAGATTTA CATATGTTTA CGGGCCGGGC TGATTTGGCT ATGTTTGATT |
| 701 | GGATTAGGTG GAGGATTAGG CCCACCCATC CTGAAAATCA GGAAGGGGTC |
| 751 | AGTATTATTA GTTTAATGAA AAGGGAGAAT TAGTACGTAA GATTTTGTAS |
| 801 | ACTTTTACGT AAGTCTAGCA TTATTGTTAA CCACCACAGT CCACGTCTCT |
| 851 | GCGTCCGCTC ATATCACCTT GCTCGATCGT CTCCTCCACA AACTTTTCTT |
| 901 | TCCGGCCGTG TGTGGATGAT AGTGTGTACT CTCTAGCAGT TGATTGAAGG |
| 951 | ATTGGACTGA GTCTAGTCGA CGCTAGTGAC CTAAGGGGAC GAAGATGCGA |
| 1001 | GGAAGGCCGG TCCTGTACTC TCTCGTCCAT GCATGTCGCG AGCTGCGTCG |
| 1051 | TCCCCATCAC CGCCACCACC ACCGCCATGG TAGGTCTCCA CCTTGGTCGA |
| 1101 | CCTCCTCCAC AGACTTTTCG CACCAATTAA TTCCGGCCAG TCGGCGACGA |
| 1151 | CCACTTCCCG TGGTGCTGGT GAATGAATTT ATGCGTGTGT GTCCTATGCT |
| 1201 | TGTCATTCCA GAGATATGCC AGTTGCGTTC TCACGGCTGA GTCATTGGCA |
| 1251 | CCTCACCTTC TGTCTCTCTC GTTAAATTTG TATCGATATA TAAGTGCTTT |
| 1301 | TGAGTACTTG CATATATAAG TGCTTTTGGA TCTAAAAAGT TATTAGTTTT |
| 1351 | CATGCTTAAG TATCTGATCA ATTTGCGGTG GTAGTGGCAT CTTTCTTGCT |
| 1401 | ATTCTGCTCT AATGAAATCT TTCACGTCCA CACGTTCTTG TTATAGATCT |
| 1451 | GCTGATTTGC TTAGATTATA AGTTCTTCTT ATTCTTCCAG ATCGATTGGA |
| 1501 | GCGACCCTCA CGCCTCTGGT GCGCCGTCGC TGTGTTCTGC TCCGCCGTGA |
| 1551 | AGAATCAAGG TGGGCTTGGT CCAGATCTAG CTAAGCTTTA ATTTCGCAGC |
| 1601 | TTGTTCAAGG CTTCACACAA TTTGGATTGC GTTACAGCTC CCTTTATTCA |
| 1651 | TCAATTTACA GGCTTCCAGC TGATTGATAC GGAGATCTCG TCCCTCCTGC |
| 1701 | TCTCATGGAT ATTGTCACCG GTGCCATTTC CAACCTGATT CCCAAGTTGG |
| 1751 | GGGAGCTGCT CACGGAGGAG TTCAAGCTGC ACAAGGGTGT CAAGAAAAAT |
| 1801 | ATTGAGGACC TCGGGAAGGA GCTTGAGAGC ATGAACGCTG CCCTCATCAA |
| 1851 | GATTGGTGAG GTGCCGAGGG AGCAGCTCGA CAGCCAAGAC AAGCTCTGGG |
| 1901 | CCGATGAGGT CAGAGAGCTC TCCTACGTCA TTGAGGATGT CGTCGACAAA |
| 1951 | TTCCTCGTAC AGGTTGATGG CATTCAGTCT GATGATAACA ACAACAAATT |
| 2001 | TAAGGGGCTC ATGAAGAGGA CGACCGAGTT GTTGAAGAAA GTCAAGCATA |
| 2051 | AGCATGGGAT AGCTCACGCG ATCAAGGACA TCCAAGAGCA ACTCCAAAAG |
| 2101 | GTGGCTGATA GGCGTGACAG GAACAAGGTA TTTGTTCCTC ATCCTACGAG |
| 2151 | ACCAATTGCT ATTGACCCTT GCCTTCGAGC TTTGTATGCT GAAGCGACAG |
| 2201 | AGCTAGTTGG CATATATGGA AAGAGGGATC AAGACCTCAT GAGGTTGCTT |
| 2251 | TCCATGGAGG GCGATGATGC CTCTAATAAG AGACTGAAGA AGGTCTCCAT |
| 2301 | TGTTGGATTT GGAGGGTTGG GCAAGACCAC TCTTGCTAGA GCGGTATACG |
| 2351 | AGAAGATTAA AGGTGATTTT GATTGTCGGG CATTTGTTCC GGTCGGTCAG |
| 2401 | AACCCTGACA TGAAGAAGGT TTTAAGGGAT ATCCTCATTG ATCTCGGAAA |
| 2451 | TCCTCACTCA GATCTTGCGA TGCTGGATGC CAATCAGCTT ATTAAAAAGC |
| 2501 | TTCATGAATT TCTAGAGAAC AAAAGGTATG CATCAATTTA GAAAAAAGTA |
| 2551 | CACTATTATG TGATGTTTGT TTCCTATGCT AGTGGAACGG ATTAGAATAT |
| 2601 | TTTTTTCATC AAGGTCACCT TTACTGGCAT AAGCAGTTCA CACTAAACAG |
| 2651 | TAAACCTTAT AGGTGAAAAA TTTCAGGCAT GTATATATAT ATATATATGT |
| 2701 | TTGATTCTTT CCGGCTTAAC AAAATAATTA GCAAGTACTT CTTGTTGCAT |
| 2751 | TTGTTCCAAC GGCTGAATTT ATTGCCACCA GTCCAAGAAA TCCATCTAAA |
| 2801 | TGTTTTACAT TTCACCAAAG TGTGTGTCAT GACAGATGTA ACAAATAATA |
| 2851 | AACCAAAAGG AGAGGAAGGA AAGAGGAAGA TAAATGTTAC AAAAATTTAA |
| 2901 | ATCAAACTTA TTTCTACCTT TCTCCTTACC TACCCAGTTG TAAAACACAT |
| 2951 | ATTATATTTT AAAGAGAGGC AACATGCGCC AAAGGCTGCC CTTGAAAATT |
| 3001 | CCTAAAATAT TGTACATTTG ACTCATGACC AAACAAAAAG TTAAATTGTC |
| 3051 | TCTTCCTTAT CGCATTATAT TTCCATGCAT GCCTTTTTCT GGAAACTTAC |
| 3101 | TATTAGCAAA ATTTAGACGA AAGGATGATG CCACATAATT TCAGTCTCCA |
| 3151 | GAGATTTGTT AGTTGCCATA TATTAAATTG GKGTGCCAAT CTATACCTGG |
| 3201 | GCCTTTTTTA TGTATCTACT TGATCATTTG AACTTCTGTA GTTAATTGTA |
| 3251 | TTCTATGAAT GATCACTCAT CCAAAAACTT GCTATTTGTG TTTCACTTTG |
| 3301 | TTGAGTCTTG AATATTTATT CATTTTGTTC ATCATACGAT TGGAGGCCCA |
| 3351 | TAATGGATGC TTAATGAGAG TAAGATTATC GAGCTCCAAA CACATGCTTC |
| 3401 | TTACTAGTGT TTGAATATAT AGCCTTATAG ATGTATAGTT CAACCCATAG |
| 3451 | ATTCATATGA CCCTCAGCTT TCTGATGTGT ATATATAACC TTACACTGAC |
| 3501 | ACTGTGAATT AATGTAGGTA TCTTGTCATA ATTGATGATA TATGGGATGA |
| 3551 | AAAATTGTGG GAAGGCATCA ACTTTGCTTT CTCCAATAGG AATAATCTAG |
| 3601 | GCAGTCGACT AATCACCACA ACCCGCATTG TCAGTGTCTC TAATTCATGT |
| 3651 | TGCTCATCAG ATGGTGATTC AGTTTATCAA ATGGAACCGC TTTCTGTTGA |
| 3701 | TGACTCTAGA ATGCTCTTCT CCAAAAGAAT ATTTCCTGAT GAGAATGGAT |
| 3751 | GTATAAATGA ATTTGAACAA GTATCCAGAG ATATTCTAAA GAAATGTGGT |
| 3801 | GGGGTACCAC TAGCCATAAT TACTATAGCT AGTGCTTTGG CTGGTGACCA |
| 3851 | GAAGATGAAA CCAAAATGTG AGTGGGATAT TCTCCTTCGG TCCCTTGGCT |
| 3901 | CTGGACTAAC AGAAGATAAC AGTTTAGAGG AGATGCGGAG AATACTCTCT |
| 3951 | TTCAGCTATT CTAATCTACC TTCGCATCTG AAAACTTGTC TACTGTATCT |
| 4001 | ATGTGTATAT CCAGAAGATA GTATGATTTC TAGAGATAAA CTGATATGGA |
| 4051 | AGTGGGTGGC TGAAGGATTT GTCCACCATG AAAATCAAGG AAATAGCCTG |
| 4101 | TATTTGCTCG GATTAAATTA CTTCAACCAG CTCATTAATA GAAGTATGAT |
| 4151 | CCAGCCAATA TATAATTATA GCGGCGAGGC ATATGCTTGC CGTGTACATG |
| 4201 | ATATGGTTCT GGACCTTATC TGCAACTTGT CATATGAAGC AAAGTTTGTG |
| 4251 | AATCTATTGG ATGGCACTGG GAATAGCATG TCTTCACAGA GTAATTGTCG |
| 4301 | CCGTTTGTCC CTTCAAAAAA GAAATGAAGA TCATCAAGTC AGGCCTTTCA |
| 4351 | CAGATATCAA GAGTATGTCA CGAGTGAGGT CAATTACTAT CTTTCCATCT |
| 4401 | GCTATTGAAG TCATGCCATC TCTTTCAAGG TTTGACGTTT TACGTGTACT |
| 4451 | TGATCTGTCA CGATGTAATC TTGGGGAGAA TAGCAGCCTG CAGCTTAACC |
| 4501 | TCAAGGATGT TGGACATTTA ACTCACCTAA GGTACCTTGG TCTAGAAGGT |
| 4551 | ACCAACATCA GTAAGCTCCC TGCTGAGATA GGAAAACTGC AGTTTTTGGA |
| 4601 | GGTGTTGGAT CTTGGAAACA ATCGTAATAT AAAGGAATTG CCGTCCACAG |
| 4651 | TTTGTAATTT CAGAAGATTA ATCTACCTAA ATTTAGTTGG CTGTCAGGTG |
| 4701 | GTTCCTCCAG TTGGTTTGTT GCAAAATCTA ACAGCCATAG AAGTGTTGAG |
| 4751 | GGGTATCTTG GTCTCTCTGA ACATTATTGC ACAACAGCTT GGCAAGTTGA |
| 4801 | AAAGTATGAG GGAGCTTGAG ATTCGCTTCA ATGATGGTAG TTTGGATTTG |
| 4851 | TATGAAGGTT TCGTGAAGTC TCTTTGCAAC TTACATCACA TAGAAAGCCT |
| 4901 | AATCATTGGT TGCAATTCTA GAGAAACATC ATCTTTTGAA GTGATGGATC |
| 4951 | TCTTGGGAGA ACGGTGGGTG CCTCCTGTAC ATCTCCGTGA ATTTGAGTCG |
| 5001 | TCCATGCCTA GCCAACTCTC TGCACTGCGA GGGTGGATAA AGAGAGACCC |
| 5051 | CTCCCATCTC TCAAACCTCT CCGACTTAGT CCTGCCAGTG AAGGAAGTGC |
| 5101 | AACAGGATGA CGTGGAAATC ATTGGGGGGT TGCTGGCCCT TCGCCGTCTC |
| 5151 | TGGATAAAGA GCAACCACCA AACACAACGG CTGCTAGTCA TCCCTGTAGA |
| 5201 | TGGGTTCCAC TGTATTGTTG ACTTTCAGTT GGACTGTGGA TCTGCCACGC |
| 5251 | AGATATTGTT TGAGCCTGGA GCTTTGCCGA GGGCAGAATC AGTTGTGATC |
| 5301 | AGTCTGGGCG TGCGGGTGGC GAAAGAGGAT GGTAACCGTG GCTTCGACTT |
| 5351 | GGGCCTGCAA GGGAACTTGC TATCCCTTCG GCGGCATGTC TTTGTTCTTA |
| 5401 | TCTATTGTGG TGGAGCGAGG GTTGGGGAGG CAAAGGAAGC GAAGGCTGCG |
| 5451 | CTGAGGCGTG CCCAGGAAGC TCATCCCGAC CATCTCCGGA TTTATATTGA |
| 5501 | CATGAGGCCG TGTATAGCAG AAGGTATCGC ATGTTGCACC TAACTAATTA |
| 5551 | CTTGTGCACT TACGCATGTG TTTTTTTTCT CAATGACCGA CTAACCTTAT |
| 5601 | TACTTTCTGT GTTGGTTTTG ATCTCTAAAT CTCCCAAGGC TCATGATGAC |
| 5651 | GATTTGTGTG AGGGCGAGGA GGAGAACTAA TTTCTGATCC AGAGCGACTC |
| 5701 | ACATTGCACA GATGTGCTCT CGAGGTAGCA GCGGCGCGGG GCGTTGGAGT |
| 5751 | TACAGCTGGT GGCATCAGAG ATGCTTGTTT CACAAACAGT TCGGGCGGGC |
| 5801 | GCTGACCATG CAAATGTTTC GAACTTTGCT GGAACTTGTG TGATGAGCTT |
| 5851 | CTTTTAAATG GCACTCAGCT TGCAGAAAGA AACATGGTTT TGTTTTGTAA |
| 5901 | TGAATAAGCA AGGGTGTTGG GGTGAATTGA TCCTTACAAG GATAGCTTTG |
| 5951 | CTTTTCTTTA GTTGAGGGCC ATCGTTGCTG CTCTGTTTTG CATGTTGTTG |
| 6001 | TTACATGGGA GGACATGCTA GTGTATTTTG TTTTTAAGCT GAGCCGAACA |
| 6051 | AACCTGAGTA TGTATTATCA GTTCCGTGTT GAATGAAATC TGAGCTCATT |
| 6101 | AATTCAATAA AAACTGTGGT TTACTGTTGG ACTTGTTACT TAAAAACTAC |
| 6151 | CCACTTCGTC CGGAATTATT AGCATTTAGA GACATCCATT TGAACCTCAG |
| 6201 | GTAGTTCTGG ACGGAGGTAG TACTATTTAC TAGTTCTACT AACATGTTTG |
| 6251 | TGTTTACATA CAAATGAAAA GTGTGATTCG AACTAACAAG TACGTACGAT |
| 6301 | TTCTAAGGTG TGCTTCCAAC TAACAAGCAT GTGTACCCCA ATGGCAGCAA |
| 6351 | ATTATTTTTG TATGTTTGAA AACGTTGTCG AAAAGCCAAA TAAAGCCTAA |
| 6401 | ATCCAACAGT GACAAAAAGG GCCAGATATT TGTGCCGATT TAACCGCGTC |
| 6451 | ATTCTCCGTA GTTTTTCATT TACTCCCTAT ATGTATTCTT ATGTGTTCCA |
| 6501 | GCTCTGTCAT ACACATAGTG AACTCAGTGG TGGTAAAAGT CGATCAAGGG |
| 6551 | AAGCATAAGC GTCGACTAGG GATGAAAATG GAGTGAAAAC TTTCCGCTTT |
| 6601 | TCTAGAGGGA AAATGAAAAG GGGGAGGAAA CATGAAAACA AAAAAAAAGG |
| 6651 | AATTTGCAAA ATGGAAGTGG AAATGGATTT TTTATGCTGA AACAGAAATA |
| 6701 | AAAACAGAAC GGTGTTTTCC AATGAACGTA CTCAACTAGA CCCTATACAC |
| 6751 | AATTGTTCAG TGAAACTTCA ATACTACGCC AAGTTGCTAA CAT |

Annex IIIb—Mla6-A GENOMIC SEQUENCE

Mla6genomic1.seq

Mla 6-A cDNA Transcript in Bold Type

This is an update of the above sequence, with changes being introduced immediately after the TAA stop codon of the open reading frame, and inclusion of an intron within the 3′ UTR.

| 1 | AACTATGTTT AAAAAACTTC CAGGAATTTT TTGACTTTTT TTTAATTTCT |
|---|---|
| 51 | AAATTATTTT TAAATTCAGG TGCACTGGAA CATGAGACTC ATTGGGTATT |
| 101 | TCCGGTGTTG ATTTGAGGAG TAATTTACCA CCTGGCAAAT GACTGCATAC |
| 151 | ACAGAGGAGT AATGCATGAT GTGGACTGAC CAACCAACTG AGGAGATTCA |
| 201 | GAGAAATGAG AGGAGAGTAA ATGCAGTGAA TGATGGCTGG TGGACGGACC |
| 251 | ATATACAGTG TATGTAATTA TTTTGCTCTG AATCCCTGTC TCTCTGTGAC |
| 301 | CCACTGAATA AACACATCAG CCAAAAGCAG TACTGTTCGG ACTTCGGAGG |
| 351 | GATCGTGGAG TAGTAGTAAT TTCCTCTCTT GACTGTTGTT CCTCTGAGTC |
| 401 | CTGTGCTCCC CGCCTCCACT GACTGCTACC TCCATCTCGT CTCAGTCCTC |
| 451 | TCCTTCATTT CAAGCTGTGA ACCGAAAACA TGCACCCAGT CCGGCCTTGA |
| 501 | TGTAATGCAG GCAACCAATC GACATGGAGA TGTCGATTTT TAGCGTATAT |
| 551 | ATGCTTAGCC AGACCCAACT AGATCAAATA TGCAAGGTAC CTGAAAACGA |
| 601 | TGCCGGTAAC CCCAAATCGC GTCGTGAACC GGAGTAATGC TAGACTTACG |
| 651 | TAAAGATTTA CATATGTTTA CGGGCCGGGC TGATTTGGCT ATGTTTGATT |
| 701 | GGATTAGGTG GAGGATTAGG CCCACCCATC CTGAAAATCA GGAAGGGGTC |
| 751 | AGTATTATTA GTTTAATGAA AAGGGAGAAT TAGTACGTAA GATTTTGTAS |
| 801 | ACTTTTACGT AAGTCTAGCA TTATTGTTAA CCACCACAGT CCACGTCTCT |
| 851 | GCGTCCGCTC ATATCACCTT GCTCGATCGT CTCCTCCACA AACTTTTCTT |
| 901 | TCCGGCCGTG TGTGGATGAT AGTGTGTACT CTCTAGCAGT TGATTGAAGG |
| 951 | ATTGGACTGA GTCTAGTCGA CGCTAGTGAC CTAAGGGGAC GAAGATGCGA |
| 1001 | GGAAGGCCGG TCCTGTACTC TCTCGTCCAT GCATGTCGCG AGCTGCGTCG |
| 1051 | TCCCCATCAC CGCCACCACC ACCGCCATGG TAGGTCTCCA CCTTGGTCGA |
| 1101 | CCTCCTCCAC AGACTTTTCG CACCAATTAA TTCCGGCCAG TCGGCGACGA |
| 1151 | CCACTTCCCG TGGTGCTGGT GAATGAATTT ATGCGTGTGT GTGCTATGCT |
| 1201 | TGTCATTCCA GAGATATGCC AGTTGCGTTC TCACGGCTGA GTCATTGGCA |
| 1251 | CCTCACCTTC TGTCTCTCTC GTTAAATTTG TATCGATATA TAAGTGCTTT |
| 1301 | TGAGTACTTG CATATATAAG TGCTTTTGGA TCTAAAAAGT TATTAGTTTT |
| 1351 | CATGCTTAAG TATCTGATCA ATTTGCGGTG GTAGTGGCAT CTTTCTTGCT |
| 1401 | ATTCTGCTCT AATGAAATCT TTCACGTCCA CACGTTCTTG TTATAGATCT |
| 1451 | GCTGATTTGC TTAGATTATA AGTTCTTCTT ATTCTTCCAG ATCGATTGGA |
| 1501 | GCGACCCTCA CGCCTCTGGT GCGCCGTCGC TGTGTTCTGC TCCGCCGTGA |
| 1551 | AGAATCAAGG TGGGCTTGGT CCAGATCTAG CTAAGCTTTA ATTTCGCAGC |
| 1601 | TTGTTCAAGG CTTCACACAA TTTGGATTGC GTTACAGCTC CCTTTATTCA |
| 1651 | TCAATTTACA GGCTTCCAGC TGATTGATAC GGAGATCTCG TCCCTCCTGC |
| 1701 | TCTCATGGAT ATTGTCACCG GTGCCATTTC CAACCTGATT CCCAAGTTGG |
| 1751 | GGGAGCTGCT CACGGAGGAG TTCAAGCTGC ACAAGGGTGT CAAGAAAAAT |
| 1801 | ATTGAGGACC TCGGGAAGGA GCTTGAGAGC ATGAACGCTG CCCTCATCAA |
| 1851 | GATTGGTGAG GTGCCGAGGG AGCAGCTCGA CAGCCAAGAC AAGCTCTGGG |
| 1901 | CCGATGAGGT CAGAGAGCTC TCCTACGTCA TTGAGGATGT CGTCGACAAA |
| 1951 | TTCCTCGTAC AGGTTGATGG CATTCAGTCT GATGATAACA ACAACAAATT |
| 2001 | TAAGGGGCTC ATGAAGAGGA CGACCGAGTT GTTGAAGAAA GTCAAGCATA |
| 2051 | AGCATGGGAT AGCTCACGCG ATCAAGGACA TCCAAGAGCA ACTCCAAAAG |
| 2101 | GTGGCTGATA GGCGTGACAG GAACAAGGTA TTTGTTCCTC ATCCTACGAG |
| 2151 | ACCAATTGCT ATTGACCCTT GCCTTCGAGC TTTGTATGCT GAAGCGACAG |
| 2201 | AGCTAGTTGG CATATATGGA AAGAGGGATC AAGACCTCAT GAGGTTGCTT |
| 2251 | TCCATGGAGG GCGATGATGC CTCTAATAAG AGACTGAAGA AGGTCTCCAT |
| 2301 | TGTTGGATTT GGAGGGTTGG GCAAGACCAC TCTTGCTAGA GCGGTATACG |
| 2351 | AGAAGATTAA AGGTGATTTT GATTGTCGGG CATTTGTTCC GGTCGGTCAG |
| 2401 | AACCCTGACA TGAAGAAGGT TTTAAGGGAT ATCCTCATTG ATCTCGGAAA |
| 2451 | TCCTCACTCA GATCTTGCGA TGCTGGATGC CAATCAGCTT ATTAAAAAGC |
| 2501 | TTCATGAATT TCTAGAGAAC AAAAGGTATG CATCAATTTA GAAAAAAGTA |
| 2551 | CACTATTATG TGATGTTTGT TTCCTATGCT AGTGGAACGG ATTAGAATAT |
| 2601 | TTTTTTCATC AAGGTCACCT TTACTGGCAT AAGCAGTTCA CACTAAACAG |
| 2651 | TAAACCTTAT AGGTGAAAAA TTTCAGGCAT GTATATATAT ATATATATGT |
| 2701 | TTGATTCTTT CCGGCTTAAC AAAATAATTA GCAAGTACTT CTTGTTGCAT |
| 2751 | TTGTTCCAAC GGCTGAATTT ATTGGCACCA GTCCAAGAAA TCCATCTAAA |
| 2801 | TGTTTTACAT TTCACCAAAG TGTGTGTCAT GACAGATGTA ACAAATAATA |
| 2851 | AACCAAAAGG AGAGGAAGGA AAGAGGAAGA TAAATGTTAC AAAAATTTAA |
| 2901 | ATCAAACTTA TTTCTACCTT TCTCCTTACC TACCCAGTTG TAAAACACAT |
| 2951 | ATTATATTTT AAAGAGAGGC AACATGCGCC AAAGGCTGCC CTTGAAAATT |
| 3001 | CCTAAAATAT TGTACATTTG ACTCATGACC AAACAAAAAG TTAAATTGTC |
| 3051 | TCTTCCTTAT CGCATTATAT TTCCATGCAT GCCTTTTTCT GGAAACTTAC |
| 3101 | TATTAGCAAA ATTTAGACGA AAGGATGATG CCACATAATT TCAGTCTCCA |
| 3151 | GAGATTTGTT AGTTGCCATA TATTAAATTG GTGTGCCAAT CTATACCTGG |
| 3201 | GCCTTTTTTA TGTATCTACT TGATCATTTG AACTTCTGTA GTTAATTGTA |
| 3251 | TTCTATGAAT GATCACTCAT CCAAAAACTT GCTATTTGTG TTTCACTTTG |
| 3301 | TTGAGTCTTG AATATTTATT CATTTTGTTC ATCATACGAT TGGAGGCCCA |
| 3351 | TAATGGATGC TTAATGAGAG TAAGATTATC GAGCTCCAAA CACATGCTTC |
| 3401 | TTACTAGTGT TTGAATATAT AGCCTTATAG ATGTATAGTT CAACCCATAG |
| 3451 | ATTCATATGA CCCTCAGCTT TCTGATGTGT ATATATAACC TTACACTGAC |
| 3501 | ACTGTGAATT AATGTAGGTA TCTTGTCATA ATTGATGATA TATGGGATGA |
| 3551 | AAAATTGTGG GAAGGCATCA ACTTTGCTTT CTCCAATAGG AATAATCTAG |
| 3601 | GCAGTCGACT AATCACCACA ACCCGCATTG TCAGTGTCTC TAATTCATGT |
| 3651 | TGCTCATCAG ATGGTGATTC AGTTTATCAA ATGGAACCGC TTTCTGTTGA |
| 3701 | TGACTCTAGA ATGCTCTTCT CCAAAAGAAT ATTTCCTGAT GAGAATGGAT |
| 3751 | GTATAAATGA ATTTGAACAA GTATCCAGAG ATATTCTAAA GAAATGTGGT |
| 3801 | GGGGTACCAC TAGCCATAAT TACTATAGCT AGTGCTTTGG CTGGTGACCA |
| 3851 | GAAGATGAAA CCAAAATGTG AGTGGGATAT TCTCCTTCGG TCCCTTGGCT |
| 3901 | CTGGACTAAC AGAAGATAAC AGTTTAGAGG AGATGCGGAG AATACTCTCT |
| 3951 | TTCAGCTATT CTAATCTACC TTCGCATCTG AAAACTTGTC TACTGTATCT |
| 4001 | ATGTGTATAT CCAGAAGATA GTATGATTTC TAGAGATAAA CTGATATGGA |
| 4051 | AGTGGGTGGC TGAAGGATTT GTCCACCATG AAAATCAAGG AAATAGCCTG |
| 4101 | TATTTGCTCG GATTAAATTA CTTCAACCAG CTCATTAATA GAAGTATGAT |
| 4151 | CCAGCCAATA TATAATTATA GCGGCGAGGC ATATGCTTGC CGTGTACATG |
| 4201 | ATATGGTTCT GGACCTTATC TGCAACTTGT CATATGAAGC AAAGTTTGTG |
| 4251 | AATCTATTGG ATGGCACTGG GAATAGCATG TCTTCACAGA GTAATTGTCG |
| 4301 | CCGTTTGTCC CTTCAAAAAA GAAATGAAGA TCATCAAGTC AGGCCTTTCA |
| 4351 | CAGATATCAA GAGTATGTCA CGAGTGAGGT CAATTACTAT CTTTCCATCT |
| 4401 | GCTATTGAAG TCATGCCATC TCTTTCAAGG TTTGACGTTT TACGTGTACT |
| 4451 | TGATCTGTCA CGATGTAATC TTGGGGAGAA TAGCAGCCTG CAGCTTAACC |
| 4501 | TCAAGGATGT TGGACATTTA ACTCACCTAA GGTACCTTGG TCTAGAAGGT |
| 4551 | ACCAACATCA GTAAGCTCCC TGCTGAGATA GGAAAACTGC AGTTTTTGGA |
| 4601 | GGTGTTGGAT CTTGGAAACA ATCGTAATAT AAAGGAATTG CCGTCCACAG |
| 4651 | TTTGTAATTT CAGAAGATTA ATCTACCTAA ATTTAGTTGG CTGTCAGGTG |
| 4701 | GTTCCTCCAG TTGGTTTGTT GCAAAATCTA ACAGCCATAG AAGTGTTGAG |
| 4751 | GGGTATCTTG GTCTCTCTGA ACATTATTGC ACAAGAGCTT GGCAAGTTGA |
| 4801 | AAAGTATGAG GGAGCTTGAG ATTCGCTTCA ATGATGGTAG TTTGGATTTG |
| 4851 | TATGAAGGTT TCGTGAAGTC TCTTTGCAAC TTACATCACA TAGAAAGCCT |
| 4901 | AATCATTGGT TGCAATTCTA GAGAAACATC ATCTTTTGAA GTGATGGATC |
| 4951 | TCTTGGGAGA ACGGTGGGTG CCTCCTGTAC ATCTCCGTGA ATTTGAGTCG |
| 5001 | TCCATGCCTA GCCAACTCTC TGCACTGCGA GGGTGGATAA AGAGAGACCC |
| 5051 | CTCCCATCTC TCAAACCTCT CCGACTTAGT CCTGCCAGTG AAGGAAGTGC |
| 5101 | AACAGGATGA CGTGGAAATC ATTGGGGGGT TGCTGGCCCT TCGCCGTCTC |
| 5151 | TGGATAAAGA GCAACCACCA AACACAACGG CTGCTAGTCA TCCCTGTAGA |
| 5201 | TGGGTTCCAC TGTATTGTTG ACTTTCAGTT GGACTGTGGA TCTGCCACGC |
| 5251 | AGATATTGTT TGAGCCTGGA GCTTTGCCGA GGGCAGAATC AGTTGTGATC |
| 5301 | AGTCTGGGCG TGCGGGTGGC GAAAGAGGAT GGTAACCGTG GCTTCGACTT |
| 5351 | GGGCCTGCAA GGGAACTTGC TATCCCTTCG GCGGCATGTC TTTGTTCTTA |
| 5401 | TCTATTGTGG TGGAGCGAGG GTTGGGGAGG CAAAGGAAGC GAAGGCTGCG |
| 5451 | CTGAGGCGTG CCCAGGAAGC TCATCCCGAC CATCTCCGGA TTTATATTGA |
| 5501 | CATGAGGCCG TGTATAGCAG AAGGTATCGC ATGTTGCACC TAACTAATTA |
| 5551 | CTTGTGCACT TACGCATGTG TTTTTTTTCT CAATGACCGA CTAACCTTAT |
| 5601 | TACTTTCTGT GTTGGTTTTG ATCTCTAAAT CTCCCAAGGT GCTCATGATG |
| 5651 | ACGATTTGTG TGAGGGCGAG GAGGAGAACTAATTTCTGAT CCAGAGCGAC |
| 5701 | TCACATTGCA TCAGATGTGC TCTCGAGGTA TGTAGCAGAT AAGAAACAGA |
| 5751 | TTAAGGTATT TACAAAAATT GCTTAGACAT AAGTATCTGA TCAGAAAAGT |
| 5801 | GGACTTGGCA GTGTAGTGTG AAACTTGCCT AGTCACTTTT TTGGCAAGGG |
| 5851 | GTGATGAAAG ATAAGAATTA TTTTATGCAA ATTGATAGAA GGATAGTCAG |
| 5901 | TAATGGGGAA TTGGGGATAT GACTAGATTT TCAGAAGTTA TATGTACAAG |
| 5951 | GAGTGTTGTT TTACCGAAAA GGCTTTCATC CCGGTTTATA TATAAAGCAA |
| 6001 | ACCACCAGAT CAAGAGTACA AGCATAAGAC CAAACCAGAC ACGCATACAC |
| 6051 | ATACCCAAGA TAGAACGACG TCAAATACGG GGGTTCTGCT CAGGGCACAG |
| 6101 | CTCAACAAGC CCTAAAAAAC AAAATAAGGC GGAGGGACCG CAATAGAAGC |
| 6151 | AACTAATCTG GCTCTGGAGG TGGTGGCGGA ACCAAGCGGA AGGCCATCAT |
| 6201 | CCGCAGATCT GCGATGATGG AGTCGATGGC GTCCTGATCC GGGAGGGGTG |
| 6251 | CTTGTTTTAG GTGACAAGTA CCCCAGGTTA TACAGTTTAT CTGTAGCTAG |
| 6301 | AGTGCTACAC GCTAATTTCA CTGCTCTAAA ATGTAAAGGG ATCTTGTATG |
| 6351 | GTGAAACTGC TGAGCTACGA CATCGATTGC TTGCTGATCG TGAAGGCTTT |
| 6401 | GGTCTTGAGG AATGAGCAAG ATTCATGTAG ATGACGCCGC AAAATAAATA |
| 6451 | TATACGTAAT GGCTTTCAAA GAATAATGGG GTATTTTCTG TGCATCCTAT |
| 6501 | ATTTATAGCT TTGAATGCTC AACAAGTGAA ATGACCATAA AGAAAATTTT |
| 6551 | GGCATGTAAA AGGTCCACTT ACGATCATAG TTTTTTATAG TTAGCATTCA |
| 6601 | GAAATAGTAT CGGCAGAGTT AATCTGAATC GTCGAGGAAT GGTAATTGCT |
| 6651 | TGAAAAATGT TTTCGCTGAG GATATTTGAT GTTTTATTGG TCTGTCTAAC |
| 6701 | AAAGAATAGA AATGCACGAT ATGTAGGTAG CAGCGGCGCG GGGCGTTGGA |
| 6751 | GTTACAGCTG GTGGCATCAG AGATGCTTGT TTCACAAACA GTTCGGGCGG |
| 6801 | GCGCTGACCA TGCAAATGTT TCGAACTTTG CTGGAACTTG TGTGATGAGC |
| 6851 | TTCTTTTAAA TGGCACTCAG CTTGCAGAAA GAAACATGGT TTTGTTTTGT |
| 6901 | AATGAATAAG CAAGGGTGTT GGGGTGAATT GATCCTTACA AGGATAGCTT |
| 6951 | TGCTTTTCTT TAGTTGAGGG CCATCGTTGC TGCTCTGTTT TGCATGTTGT |
| 7001 | TGTTACATGG GAGGACATGC TAGTGTATTT TGTTTTTAAG CTGAGCCGAA |
| 7051 | CAAACCTGAG TATGTATTAT CAGTTCCGTG TTGAATGAAA TCTGAGCTCA |
| 7101 | TTAATTCAAT AAAAACTGTG GTTTACTGTT GGACTTGTTA CTTAAAAACT |
| 7151 | ACC |
| Annex IV - Mla12 CDNA SEQUENCE | |
| Mla12cDNA.seq Length: 3434 | |
| 1 | CTGACACCCG TGGATCTAAA AAGTTATTAG TTTTCATGCT TAAGTATCTG |
| 51 | ATCAATTTGC GGTGATCGAT TGGAGCGATC GTCACGCCTC TGGTGCGCCG |
| 101 | TCGCTGTGTT CTGCTCCGCC GTGAAGAATC AAGGCTTCCA GCTGATTATA |
| 151 | GGGCTGATTG ATACGGATAT CTCGTCCTCC AGCTCTCATG GATATTGTCA |
| 201 | CCGGTGCCAT TTCCAACCTG ATTCCCAAGT TGGGGGAGCT ACTCACGGAG |
| 251 | GAGTTCAAGC TGCACAAGGG TGTCAAGAAA AATATTGAGG ACCTCGGGAA |
| 301 | GGAGCTTGAG AGCATGAACG CTGCCCTCAT CAAGATTGGT GAGGTGCCGA |
| 351 | GGGAGCAGCT CGACAGCCAA GACAAGCTCT GGGCCGATGA GGTCAGAGAG |
| 401 | CTCTCCTACG TCATTGAGGA TGTCGTCGAC AAGTTCCTCG TACAGGTTGA |
| 451 | TGGCATTAAG TCTGATGATA ACAACAACAA ATCTAAGGGG CTCATGAAGA |
| 501 | GGACTACCGA GTTGTTGAAG AAAGTCAAGC ATAAGCATGG GATAGCTCAC |
| 551 | GCGATCAAGG ACATCCAAGA GCAACTCCAA AAGGTGGCTG ATAGGCGTGA |
| 601 | CAGGAACAAG GTATTTGTTC CTCATCCTAC GAGAACAATT GCTATTGACC |
| 651 | CTTGCCTTCG AGCTTTGTAT GCTGAAGCGA CAGAGCTAGT TGGCATATAT |
| 701 | GGAAAGAGGG ATCAAGGCCT CATGAGGTTG CTTTCCATGG AGGGCGATGA |
| 751 | TGCCTCTAAT AAGAGACTGA AGAAGGTCTC CATTGTTGGA TTTGGAGGGT |
| 801 | TGGGCAAGAC CACTCTTGCT AGAGCGGTAT ACGAGAAGAT TAAAGGTGAT |
| 851 | TTCGATTGTC GGGCATTTGT TCCGGTCGGT CAGAACCCTG ACATGAAGAA |
| 901 | GGTTTTAAGG GATATCCTCA TTGATCTCGG AAATCCTCAC TCAGATCTTG |
| 951 | CGATGCTGGA TGCCAATCAG CTTATTAAAA AGCTTCATGA ATTTCTAGAG |
| 1001 | AACAAAAGGT ATCTTGTCAT AATTGATGAT ATATGGGATG AAAAATTGTG |
| 1051 | GGAAGGCATC AACTTTGCTT TCTCCAATAG GAATAATCTA GGCAGTCGGC |
| 1101 | TAATCACCAC AACCCGCATT GTCAGTGTCT CTAATTCATG TTGCTCATCA |
| 1151 | GATCGTGATT CAGTTTATCA AATGGAACCG CTTTCTGTTG ATGACTCCAG |
| 1201 | AATGCTCTTC TACAAAAGAA TATTTCCTGA TGAGAATGCA TGTATAAATG |
| 1251 | AATTTGAACA AGTATCCAGA GATATTCTAA AGAAATGTGG TGGGGTACCA |
| 1301 | CTAGCCATAA TTACTATAGC TAGTGCTTTG GCTGGTGACC AGAAGATGAA |
| 1351 | ACCAAAATGT GAGTGGGATA TTCTCCTTCG GTCCCTTGGC TCTGGACTAA |
| 1401 | CAGAAGATAA CAGTTTAGAG GAGATGCGGA GAATACTCTC TTTCAGCTAT |
| 1451 | TCTAATCTAC CTTCGAATCT GAAAACTTGT CTACTGTATC TATGTGTATA |
| 1501 | TCCAGAAGAT AGTATGATTT CTAGAGATAA ACTGATATGG AAGTGGGTGG |
| 1551 | CCGAAGGATT TGTCCACCAT GAAAATCAAG GAAATAGCCT GTATTTGCTC |
| 1601 | GGATTAAATT ACTTCAACCA GCTCATTAAT AGAAGTATGA TCCAGCCAAT |
| 1651 | ATATAATTAT AGCGGCGAGG CATATGCTTG CCGTGTACAT GATATGGTTC |
| 1701 | TGGACCTTAT CTGCAACTTG TCACGTGAAG CAAAGTTTGT GAATCTATTG |
| 1751 | GATGGCACTG GGAATAGCAT GTCTTCACAG AGTAATTGTC GTCGTTTGTC |
| 1601 | CCTTCAGAAA AGAAATGAAG ATCATCAAGC CAGGCCTCTC ATAGATATCA |
| 1851 | AGAGTATGTC ACGAGTGAGG TCAATTACTA TCTTTCCACC TGCTATTGAA |
| 1901 | GTCATGCCAT CTCTTTCAAG GTTTGAGGTT TTATGTGTAC TTGATTTGTC |
| 1951 | GAAATGTAAT CTTGGGGAGG ATAGCAGCCT GCAACTTAAC CTCAAGGATG |
| 2001 | TTGGACAATT AATTCAGCTA AGGTACCTTG GTCTAGAATG TACCAATATA |
| 2051 | AGTAAGCTCC CGACTGAGAT AGGAAAACTG CAGTTTTTGG AGGTGTTGGA |
| 2101 | TCTTGGAAAC AATCCTAATC TAAAAGAATT GCCGTCCACT ATTCGTAATT |
| 2151 | TCAGAAGATT AATCTACCTA AATTTAGTTG GCTGTCAGGT GATTCCTCCA |
| 2201 | GTGGGTGTGT TGCAAAATCT GACATCCATA GAAGTATTGA GGGGTATCTT |
| 2251 | GGTCTATCTG AACATTATTG CACAAGAGCT TGGCAACCTG GAAAGGGTGA |
| 2301 | GAGATCTTGA GATTCGCTTC AATGATGGTA GTTTGGATTT GTATGAAGGT |
| 2351 | TTGGTGAATT CTCTGTGCAA CCTACATCAC ATCGAAAGTC TAAATATTCG |
| 2401 | TTGCAATCCC GGAGAAACAT CATCTTTTGA ACTGATGGAT CTCTTGGAAG |
| 2451 | AACGTTGGGT GCCGCCTGTA CATCTCCGTG AATTTAAGTC ATTCATGCCC |
| 2501 | AGCCAACTCT CTGCACTGCG AGGGTGGATA CAGAGAGACC CCTCCCATCT |
| 2551 | CTCGAACCTC TCCGAGTTAA CCCTCTGGCC AGTGAAGGAC GTGCAGCAGG |
| 2601 | ATGACGTGGA AATCATTGGG GGGTTGTTGT CCCTTCGTCG TCTCTGGATA |
| 2651 | GTAAAGAGCA TCCACCAAAC GCAACGGCTG CTAGTCATCC GTGCAGATGG |
| 2701 | GTTCCGCTCT ATGGTTGAAT TTCGTTTGGA TTGTGGATCT GCCACGCAGA |
| 2751 | TATTGTTTGA GCCAGGAGCT TTGCCGAGGG CGGAATCAGT TGTGATCAGT |
| 2801 | CTGGGCGTGC GGGTGGCGAA AGAGGATGGT AACCGTGGCT TCCACTTGGG |
| 2851 | CCTGCAGGAA GCAAAGGATG TCTCCCTTCG GTGGGATGTC TTTGTTCTTC |
| 2901 | TCTATTGTGG TGGAGCGAGG GTTGGGGAGG CAAAGGAAGC GGAGGCTGCG |
| 2951 | GTGAGGCGTG CCCTGGAAGC TCATCCCAGA CATCCTCGGA TTTATATTGA |
| 3001 | CATGAGGCCG GATATACAGG AAGGTGCTCA TGATCACGAT TTGTGTGAGA |
| 3051 | ACGAGGACGA GGGTGAGAAC TGATTTTTGG CGCAGAAGGA TTCACACTGC |
| 3101 | ATCAGGACTG CTCTCGGTAG CAGGGGTGCG GGGTTACTGC TGGAGGCATC |
| 3151 | GGACATGCAT GTTTCACAAA CATTTTGGAT GGGTGCCGAC CGGGCGAAGA |
| 3201 | ATTGAAGGAT GGAAAGTTTT CGAACTTTTC TGAAAGTTGG GTGATGAGCT |
| 3251 | TCTTTTAAAT GGCAGTCGCT TGCCGAAAGA GATACTGCTT GGTTCTGTAA |
| 3301 | TGAATAAGTA ACGGTGTTGG ATCGAATTGA TCCTTACAAG TATATCTTTG |
| 3351 | CTTTTCTTCT GCCTGCAAAT CGCTCCCCAT TTCACCCAAT TGTAAATATG |
| 3401 | CTAACTCCAG CAAAGACCTT GATGAATCTT TGGG |
| Annex V - Mla12 POLYPEPTIDE SEQUENCE | |
| Mla12cDNA.pep Length: 962 | |
| 1 | MDIVTGAISN LIPKLGELLT EEFKLHKGVK KNIEDLGKEL ESMNAALIKI |
| 51 | GEVPREQLDS QDKLWADEVR ELSYVIEDVV DKFLVQVDGI KSDDNNNKSK |
| 101 | GLMKRTTELL KKVKHKHGIA HAIKDIQEQL QKVADRRDRN KVFVPHPTRT |
| 151 | IAIDPCLRAL YAEATELVGI YGKRDQGLMR LLSMEGDDAS NKRLKKVSIV |
| 201 | CFGGLGKTTL ARAVYEKIKG DFDCRAFVPV GQNPDMKKVL RDILIDLGNP |
| 251 | HSDLAMLDAN QLIKKLHEFL ENKRYLVIID DIWDEKLWEG INFAFSNRNN |
| 301 | LGSRLITTTR IVSVSNSCCS SDGDSVYQME PLSVDDSRML FYKRIFPDEN |
| 351 | ACINEFEQVS RDILKKCGGV PLAIITIASA LAGDQKMKPK CEWDILLRSL |
| 401 | GSGLTEDNSL EEMRRILSFS YSNLPSNLKT CLLYLCVYPE DSMISRDKLI |
| 451 | WKWVAEGFVH HENQGNSLYL LGLNYFNQLI NRSMIQPIYN YSGEAYACRV |
| 501 | HDMVLDLICN LSREAKFVNL LDGTGNSMSS QSNCRRLSLQ KRNEDHQARP |
| 551 | LIDIKSMSRV RSITIFPPAI EVMPSLSRFE VLCVLDLSKC NLGEDSSLQL |
| 601 | NLKDVGQLIQ LRYLGLECTN ISKLPTEIGK [symbol] QFLEVLDLG NNPNLKELPS |
| 651 | TIRNFRRLIY LNLVGCQVIP PVGVLQNLTS IEVLRGILVY LNIIAQELGN |
| 701 | LERVRDLEIR FNDGSLDLYE GLVNSLCNLH HIESLNIRCN PGETSSFELM |
| 751 | DLLEERWVPP VHLREFKSFM PSQLSALRGW IQRDPSHLSN LSELTLWPVK |
| 801 | DVQQDDVEII GGLLSLRRLW IVKSIHQTQR LLVIRADGFR SMVEFRLDCG |
| 851 | SATQILFEPG ALPRAESVVI SLGVRVAKED GNRGFHLGLQ EAKDVSLRWD |
| 901 | VFVLLYCGGA RVGEA [symbol] EAEA AVRRALEAHP RHPRIYIDMR PDIQEGAHDD |
| 951 | DLCENEDEGE N★ |
| 631 | L→R(L: Leucine R: Arginine) |
| 916 | K→M(K: Lysine M: Methionine) |

1 An isolated nucleic acid molecule which nucleic acid comprises an Mla nucleotide sequence derived from an Mla locus encoding an MLA polypeptide which is capable of recognising and activating a race specific defence response in a plant into which the nucleic acid is introduced and expressed, in response to challenge with a cognate Erysiphe graminis isolate.

2 A nucleic acid as claimed in claim 1 wherein the Mla locus is the Mla1 locus of Hordeum vulgare CI-16137 or the Mla6 locus of Hordeum vulgare CI-16151 or the Mla12 locus from Hordeum vulgare cultivar Sultan-5.

3 An isolated nucleic acid molecule which nucleic acid comprises an Mla nucleotide sequence which:
(i) encodes an MLA resistance polypeptide shown in FIG. 10 or Annex V, or
(ii) encodes a variant resistance polypeptide which is a homologous variant of an MLA resistance polypeptide shown in FIG. 10 or Annex V, and which shares at least about 70%, 80% or 90% identity therewith.

4 A nucleic acid as claimed in any one of claims 1 to 3 wherein the Mla nucleotide sequence is selected from a list consisting of:
Mla1 nucleotide sequence of FIG. 3; Mla1-2 nucleotide sequence of FIG. 4; Mla6 ORF of Annex I; Mla6 cDNA of Annex II; Mla6 gDNA of Annex III; Mla nucleotide sequence of FIG. 9; Mla12 cDNA of Annex IV; Mla6 gDNA of FIG. 11; a sequence which is degeneratively equivalent to any of these.

5 A nucleic acid as claimed in claim 3 wherein the Mla nucleotide sequence encodes a derivative of an MLA resistance polypeptide shown in FIG. 10 or Annex V by way of addition, insertion, deletion or substitution of one or more amino acids.

6 A nucleic acid as claimed in any one of claims 1 to 3 wherein the Mla nucleotide sequence consists of an allelic, paralogous or orthologous variant of an Mla nucleotide sequence of FIG. 9 or FIG. 11.

7 An isolated nucleic acid which comprises a nucleotide sequence which is the complement of the Mla nucleotide sequence of any one of the preceding claims.

8 An isolated nucleic acid for use as a probe or primer, said nucleic acid consisting of a distinctive sequence of at least about 16-24 nucleotides in length, which sequence is (i) conserved between the Mla1 and Mla6 nucleotide sequences of FIG. 9, but not conserved with the other sequences shown therein, or conserved between at least two of the Mla1, Mla6 or Mla12 sequences of FIG. 11 (ii) a sequence degeneratively equivalent to said conserved sequence, or (iii) the complement sequence of either.

9 A nucleic acid primer as claimed in claim 8, selected from:

| | |
|---|---|
| forward primer: | 5′TATTGTCACCGGTGCCATTC-3′ |
| reverse primer: | 5′CTCATGATGACGATTTGTGTG-3′. |

10 A method for isolating, identifying or locating a functional Mla allele, which method comprises the steps of:
(a) providing a preparation of nucleic acid from plant cells believed to encode the allele,
(b) identifying the presence of an Mla (AT)ₙ micro-satellite in the nucleic acid preparation,
(c) correlating the presence of an Mla (AT)ₙ micro-satellite in the preparation with the presence of a functional Mla allele.

11 A method as claimed in claim 10 wherein step (b) comprises the step of contacting nucleic acid in said preparation with a probe or primer adapted to identify the presence of an Mla (AT)ₙ micro-satellite in the nucleic acid preparation.

12 A method as claimed in claim 11 wherein the Mla (AT)ₙ micro-satellite sequence includes at least about 6, 8, 10, 12, 14, 16, 20, 24, 28, 32, 36, 40 or more AT repeats.

13 A method as claimed in claim 11 wherein the presence of the Mla (AT)ₙ micro-satellite sequence is determined in a product amplified from the nucleic acid preparation.

14 A nucleic acid primer for use in the method of claim 13, selected from:

| | |
|---|---|
| 1. MlaATS1 | 5′-ACTGGCATAAGCAGTTCACACTAAAC-3′ |
| 2. MlaATAS1 | 5′-CATTTATCTTCCTCTTTCCTTCCTCTCC-3′ |

15 A method for identifying, cloning, or determining the presence within a plant of a nucleic acid as claimed in claim 2 or claim 6, which method employs a nucleic acid as claimed in claim 4, 8, 9 or 14.

16 A method as claimed in claim 15, which method comprises the steps of:
(a) providing a preparation of nucleic acid from a plant cell;
(b) providing a nucleic acid molecule which is a nucleic acid as claimed in claim 4, claim 8, claim 9 or claim 14,
(c) contacting nucleic acid in said preparation with said nucleic acid molecule under conditions for hybridisation, and,
(d) identifying nucleic acid in said preparation which hybridises with said nucleic acid molecule.

17 A method as claimed in claim 15, which method comprises the steps of:
(a) providing a preparation of nucleic acid from a plant cell;
(b) providing a pair of nucleic acid molecule primers suitable for PCR, at least one of said primers being a primer of claim 8, claim 9, or claim 14
(c) contacting nucleic acid in said preparation with said primers under conditions for performance of PCR,
(d) performing PCR and determining the presence or absence, and optionally the sequence, of an amplified PCR product.

18 A recombinant vector which comprises the nucleic acid of any one of claims 1 to 6.

19 A vector as claimed in claim 18 wherein the nucleic acid is operably linked to a promoter for transcription in a host cell, wherein the promoter is optionally an inducible promoter.

20 A vector as claimed in claim 18 or claim 19 which is a plant vector.

21 A method which comprises the step of introducing the vector of any one of claims 18 to 20 into a host cell, and optionally causing or allowing recombination between the vector and the host cell genome such as to transform the host cell.

22 A host cell containing or transformed with a heterologous vector of any one of claims 18 to 20.

23 A method for producing a transgenic plant, which method comprises the steps of:
(a) performing a method as claimed in claim 21 wherein the host cell is a plant cell,
(b) regenerating a plant from the transformed plant cell.

24 A transgenic plant which is optionally selected from a species which is susceptible to powdery mildew, and which is obtainable by the method of claim 23, or which is a clone, or selfed or hybrid progeny or other descendant of said transgenic plant, which in each case includes a heterologous nucleic acid of any one of claims 1 to 6.

25 A part of propagule from a plant as claimed in claim 24, and which in either case includes a heterologous nucleic acid of any one of claims 1 to 6.

26 An isolated polypeptide which is encoded by the Mla nucleotide sequence of any one of claims 1 to 6.

27 A polypeptide as claimed in claim 26 which is an MLA resistance polypeptide shown in FIG. 5, FIG. 10 or Annex V.

28 A method of making the polypeptide of claim 26 or claim 27, which method comprises the step of causing or allowing expression from a nucleic acid of any one of claims 1 to 6 in a suitable host cell.

29 A polypeptide which comprises the antigen-binding site of an antibody having specific binding affinity for the polypeptide of claim 27.

30 A method for influencing or affecting the degree of resistance of a plant to a powdery mildew, which method comprises the step of causing or allowing expression of a heterologous nucleic acid as claimed in any one of claims 1 to 7 within the cells of the plant, following an earlier step of introducing the nucleic acid into a cell of the plant or an ancestor thereof.

31 A method as claimed in claim 30 for increasing a plant's powdery mildew disease resistance, wherein the nucleic acid is a nucleic acid as claimed in any one of claims 1 to 6.

32 A method as claimed in claim 31 which further comprises the step of manipulating a Rar1 and/or Rar2 gene in the plant.

33 An isolated nucleic acid molecule encoding the promoter or other UTR (3′ or 5′) of an Mla gene of claim 2, or a homologous variant thereof which has promoter activity.

34 A method for assessing the ability of nucleic acid encoding a putative resistance (R) gene to confer resistance against a pathogen expressing a cognate Avr gene, the method comprising the steps of:
(a) selecting plant material which comprises plant cells which express a recessive gene conferring resistance against the pathogen,
(b) introducing into the plant material, nucleic acid encoding (i) a detectable marker, (ii) a dominant susceptibility gene which inhibits the resistance conferred by the recessive gene, and (iii) the putative R gene,
(c) challenging the plant material with the pathogen,
(d) observing cells in the plant material in which the marker is expressed to determine the amount of pathogen growth present, and
(e) correlating the amount of pathogen growth with the ability of the R gene to confer resistance against the pathogen.

35 A method as claimed in claim 34 wherein the amount of pathogen growth in step (d) is determined by comparison with cells in the plant material in which the marker is expressed and which have been challenged with pathogen but in which no pathogen growth is established.

36 A method as claimed in claim 34 or claim 35 further comprising step (f) correlating the number of cells in the plant material expressing the marker with the ability of the R gene to confer a hyper-sensitive resistance response against the pathogen.

37 A method as claimed in any one of claims 34 to 36 wherein the amount of pathogen for step (e) or the number of cells for step (f) is further compared against a corresponding control system in which either (1) no R gene is present, or (2) a corresponding pathogen not expressing a cognate AVR gene is used.

38 A method as claimed in any one of claims 34 to 37 wherein the nucleic acid introduced in step (b) is introduced as a first vector encoding (i) the detectable marker, (ii) the dominant susceptibility gene and a second vector encoding (iii) the putative R gene.

39 A method as claimed in claim 38 wherein the first and second vectors are co-introduced into the plant material such that they are at least transiently expressed therein.

40 A method as claimed in any one of claims 34 to 39 wherein the marker in step (b) is selected from: Green Fluorescent Protein (GFP); GUS.

41 A method as claimed in any one of claims 34 to 40 wherein the recessive gene of step (a) provides a broad resistance against the pathogen.

42 A method as claimed in claim 41 wherein the recessive gene of step (a) is the mlo gene, and the dominant susceptibility of (b) is the Mlo gene.

43 A method as claimed in any one of claims 34 to 42 wherein the R gene is selected from an Mla gene of claim 1, and the pathogen expressing the cognate Avr gene is the cognate Erysiphe graminis isolate.

44 A method as claimed in claim 43 wherein the R gene is selected from Mla6 and the isolate is A6, or the R gene is Mla1 and the isolate is k1

45 A method as claimed in any one of claims 34 to 44 for use in the identification of a putative pathogen expressing a cognate AVR gene for a selected R gene, or a putative inhibitor of the interaction between a selected pathogen expressing a cognate AVR gene and a selected R gene.

46 A plant vector for use in a method as claimed in any one of claims 34 to 45, which vector comprises: (i) a detectable marker, (ii) a dominant susceptibility gene which inhibits the resistance conferred by the recessive gene.

47 A vector as claimed in claim 46 which is selected from: pUGLUM (FIG. 2) or pUGUS.

48 A composition of matter comprising a first vector as claimed in claim 46 or claim 47; and a second vector encoding (iii) the putative R gene.

49 A kit for assessing the ability of nucleic acid encoding a resistance (R) gene to confer resistance against a pathogen expressing a cognate Avr gene, the kit comprising: a vector or composition of any one of claims 46 to 48; one or more further materials for performing a method of any one of claims 34 to 45.


SEQUENCE LISTING (68 sequences)

SEQ ID NO: 1 - DNA, 20 bp, Artificial Sequence
  Primer
tattgtcaccggtgccattc

SEQ ID NO: 2 - DNA, 21 bp, Artificial Sequence
  Primer
ctcatgatgacgatttgtgtg

SEQ ID NO: 3 - DNA, 26 bp, Artificial Sequence
  Primer
actggcataagcagttcacactaaac

SEQ ID NO: 4 - DNA, 28 bp, Artificial Sequence
  Primer
catttatcttcctctttccttcctctcc

SEQ ID NO: 5 - DNA, 28 bp, Artificial Sequence
  Primer
aattcgatatcggtaccaagcggccgcg

SEQ ID NO: 6 - DNA, 28 bp, Artificial Sequence
  Primer
aattcgccgccgcttggatccgatatcg

SEQ ID NO: 7 - DNA, 22 bp, Artificial Sequence
  Primer
taatgagcattgcatgtctaag

SEQ ID NO: 8 - DNA, 22 bp, Artificial Sequence
  Primer
tgcagaagtaacaccaaacaac

SEQ ID NO: 9 - DNA, 23 bp, Artificial Sequence
  Primer
cagcctcttgctgagtggagatg

SEQ ID NO: 10 - DNA, 22 bp, Artificial Sequence
  Primer
tcttgcccaaccctccaaatcc

SEQ ID NO: 11 - DNA, 23 bp, Artificial Sequence
  Primer
ggttaccatcctctttcgtcacc

SEQ ID NO: 12 - DNA, 25 bp, Artificial Sequence
  Primer
ggaggctcgttgtgtctctgaatac

SEQ ID NO: 13 - DNA, 21 bp, Artificial Sequence
  Primer
tggttccaactggtgtgttgc

SEQ ID NO: 14 - DNA, 20 bp, Artificial Sequence
  Primer
ccccaatgatttccacgtcc

SEQ ID NO: 15 - DNA, 24 bp, Artificial Sequence
  Primer
gctctctcactgttcgtatggacc

SEQ ID NO: 16 - DNA, 22 bp, Artificial Sequence
  Primer
agcagctaccaggctgtattgc

SEQ ID NO: 17 - DNA, 22 bp, Artificial Sequence
  Primer
tgctttacctcaagttggctgc

SEQ ID NO: 18 - DNA, 22 bp, Artificial Sequence
  Primer
cgaaggtgtgtgatttcgatgc

SEQ ID NO: 19 - DNA, 18 bp, Artificial Sequence
  Primer
aagcatgggatagctcac

SEQ ID NO: 20 - DNA, 18 bp, Artificial Sequence
  Primer
cccaagattacatcgtga

SEQ ID NO: 21 - DNA, 24 bp, Artificial Sequence
  Primer
gcacgaggtcattccagagatatg

SEQ ID NO: 22 - DNA, 19 bp, Artificial Sequence
  Primer
gaaagagagtattctccgc

SEQ ID NO: 23 - DNA, 22 bp, Artificial Sequence
  Primer
cacctcaccttctgtctctctc

SEQ ID NO: 24 - DNA, 23 bp, Artificial Sequence
  Primer
gcatctttcttgctattctgctc

SEQ ID NO: 25 - DNA, 22 bp, Artificial Sequence
  Primer
tgccatttccaacctgattccc

SEQ ID NO: 26 - DNA, 22 bp, Artificial Sequence
  Primer
tctccctctttccttcctctcc

SEQ ID NO: 27 - DNA, 25 bp, Artificial Sequence
  Primer
cctttaatcttctcgtataccgctc

SEQ ID NO: 28 - DNA, 24 bp, Artificial Sequence
  Primer
tgtttagtgtgaactgcttatgcc

SEQ ID NO: 29 - DNA, 22 bp, Artificial Sequence
  Primer
ccttgttcctgtcacgcctatc

SEQ ID NO: 30 - DNA, 28 bp, Artificial Sequence
  Primer
gatgcttaatgagagtaagattatcgag

SEQ ID NO: 31 - DNA, 25 bp, Artificial Sequence
  Primer
gaagggacaaacgacgacaattact

SEQ ID NO: 32 - DNA, 26 bp, Artificial Sequence
  Primer
ggcatcaactttgctttctccaatag

SEQ ID NO: 33 - DNA, 24 bp, Artificial Sequence
  Primer
cgacgacaattactctgtgaagac

SEQ ID NO: 34 - DNA, 22 bp, Artificial Sequence
  Primer
taacagtttagaggagatgcgg

SEQ ID NO: 35 - DNA, 22 bp, Artificial Sequence
  Primer
atggagaaaggaaggtaggtgg

SEQ ID NO: 36 - DNA, 22 bp, Artificial Sequence
  Primer
ttagaggagatgcggagaatac

SEQ ID NO: 37 - DNA, 22 bp, Artificial Sequence
  Primer
ctcccgactgagataggaaaac

SEQ ID NO: 38 - DNA, 25 bp, Artificial Sequence
  Primer
cacaatagagaagaacaaagacatc

SEQ ID NO: 39 - DNA, 24 bp, Artificial Sequence
  Primer
ttgttgtcccttcgtcgtctctgg

SEQ ID NO: 40 - DNA, 24 bp, Artificial Sequence
  Primer
tgtgcgccaaaaatcagttctcac

SEQ ID NO: 41 - DNA, 2871 bp, Hordeum vulgare
atggatattgtcaccggtgccatttccaacctgattcccaagttgggggagctgctcacggaggagttcaagctgcacaagggtgtcaagaaaaatattgaggacctcgggaaggagcttgagagcatgaacgctgccctcatcaagattggtgaggtgccgagggagcagctcgacagccaagacaagctctgggccgatgaggtcagagagctctcctacgtcattgaggatgtcgtcgacaaattcctcgtacaggttgatggcattcagtctgatgataacaacaacaaatttaaggggctcatgaagaggacgaccgagttgttgaagaaagtcaagcataagcatgggatagctcacgcgatcaaggacatccaagagcaactccaaaaggtggctgataggcgtgacaggaacaaggtatttgttcctcatcctacgagaccaattgctattgacccttgccttcgagctttgtatgctgaagcgacagagctagttggcatatatggaaagagggatcaagacctcatgaggttgctttccatggagggcgatgatgcctctaataagagactgaagaaggtctccattgttggatttggagggttgggcaagaccactcttgctagagcggtatacgagaagattaaaggtgattttgattgtcgggcatttgttccggtcggtcagaaccctgacatgaagaaggttttaagggatatcctcattgatctcggaaatcctcactcagatcttgcgatgctggatgccaatcagcttattaaaaagcttcatgaatttctagagaacaaaaggtatcttgtcataattgatgatatatgggatgaaaaattgtgggaaggcatcaactttgctttctccaataggaataatctaggcagtcgactaatcaccacaacccgcattgtcagtgtctctaattcatgttgctcatcagatggtgattcagtttatcaaatggaaccgctttctgttgatgactctagaatgctcttctccaaaagaatatttcctgatgagaatggatgtataaatgaatttgaacaagtatccagagatattctaaagaaatgtggtggggtaccactagccataattactatagctagtgctttggctggtgaccagaagatgaaaccaaaatgtgagtgggatattctccttcggtcccttggctctggactaacagaagataacagtttagaggagatgcggagaatactctctttcagctattctaatctaccttcgcatctgaaaacttgtctactgtatctatgtgtatatccagaagatagtatgatttctagagataaactgatatggaagtgggtggctgaaggatttgtccaccatgaaaatcaaggaaatagcctgtatttgctcggattaaattacttcaaccagctcattaatagaagtatgatccagccaatatataattatagcggcgaggcatatgcttgccgtgtacatgatatggttctggaccttatctgcaacttgtcatatgaagcaaagtttgtgaatctattggatggcactgggaatagcatgtcttcacagagtaattgtcgccgtttgtcccttcaaaaaagaaatgaagatcatcaagtcaggcctttcacagatatcaagagtatgtcacgagtgaggtcaattactatctttccatctgctattgaagtcatgccatctctttcaaggtttgacgttttacgtgtacttgatctgtcacgatgtaatcttggggagaatagcagcctgcagcttaacctcaaggatgttggacatttaactcacctaaggtaccttggtctagaaggtaccaacatcagtaagctccctgctgagataggaaaactgcagtttttggaggtgttggatcttggaaacaatcgtaatataaaggaattgccgtccacagtttgtaatttcagaagattaatctacctaaatttagttggctgtcaggtggttcctccagttggtttgttgcaaaatctaacagccatagaagtgttgaggggtatcttggtctctctgaacattattgcacaagagcttggcaagttgaaaagtatgagggagcttgagattcgcttcaatgatggtagtttggatttgtatgaaggtttcgtgaagtctctttgcaacttacatcacatagaaagcctaatcattggttgcaattctagagaaacatcatcttttgaagtgatggatctcttgggagaacggtgggtgcctcctgtacatctccgtgaatttgagtcgtccatgcctagccaactctctgcactgcgagggtggataaagagagacccctcccatctctcaaacctctccgacttagtcctgccagtgaaggaagtgcaacaggatgacgtggaaatcattggggggttgctggcccttcgccgtctctggataaagagcaaccaccaaacacaacggctgctagtcatccctgtagatgggttccactgtattgttgactttcagttggactgtggatctgccacgcagatattgtttgagcctggagctttgccgagggcagaatcagttgtgatcagtctgggcgtgcgggtggcgaaagaggatggtaaccgtggcttcgacttgggcctgcaagggaacttgctatcccttcggcggcatgtctttgttcttatctattgtggtggagcgagggttggggaggcaaaggaagcgaaggctgcgctgaggcgtgcccaggaagctcatcccgaccatctccggatttatattgacatgaggccgtgtatagcagaaggtgctcatgatgacgatttgtgtgagggcgaggaggagaactaa

SEQ ID NO: 42 - DNA, 3717 bp, Artificial Sequence
  MLA6-A cDNA
gtcattccagagatatgccagttgcgttctcacggctgagtcattggcacctcaccttctgtctctctcgttaaatttgtatcgatatataagtgcttttgagtacttgcatatataagtgcttttggatctaaaaagttattagttttcatgcttaagtatctgatcaatttgcggtggtagtggcatctttcttgctattctgctctaatgaaatctttcacgtccacacgttcttgttatagatctgctgatttgcttagattataagttcttcttattcttccagatcgattggagcgaccctcacgcctctggtgcgccgtcgctgtgttctgctccgccgtgaagaatcaaggcttccagctgattgatacggagatctcgtcctcctgctctcatggatattgtcaccggtgccatttccaacctgattcccaagttgggggagctgctcacggaggagttcaagctgcacaagggtgtcaagaaaaatattgaggacctcgggaaggagcttgagagcatgaacgctgccctcatcaagattggtgaggtgccgagggagcagctcgacagccaagacaagctctgggccgatgaggtcagagagctctcctacgtcattgaggatgtcgtcgacaaattcctcgtacaggttgatggcattcagtctgatgataacaacaacaaatttaaggggctcatgaagaggacgaccgagttgttgaagaaagtcaagcataagcatgggatagctcacgcgatcaaggacatccaagagcaactccaaaaggtggctgataggcgtgacaggaacaaggtatttgttcctcatcctacgagaccaattgctattgacccttgccttcgagctttgtatgctgaagcgacagagctagttggcatatatggaaagagggatcaagacctcatgaggttgctttccatggagggcgatgatgcctctaataagagactgaagaaggtctccattgttggatttggagggttgggcaagaccactcttgctagagcggtatacgagaagattaaaggtgattttgattgtcgggcatttgttccggtcggtcagaaccctgacatgaagaaggttttaagggatatcctcattgatctcggaaatcctcactcagatcttgcgatgctggatgccaatcagcttattaaaaagcttcatgaatttctagagaacaaaaggtatcttgtcataattgatgatatatgggatgaaaaattgtgggaaggcatcaactttgctttctccaataggaataatctaggcagtcgactaatcaccacaacccgcattgtcagtgtctctaattcatgttgctcatcagatggtgattcagtttatcaaatggaaccgctttctgttgatgactctagaatgctcttctccaaaagaatatttcctgatgagaatggatgtataaatgaatttgaacaagtatccagagatattctaaagaaatgtggtggggtaccactagccataattactatagctagtgctttggctggtgaccagaagatgaaaccaaaatgtgagtgggatattctccttcggtcccttggctctggactaacagaagataacagtttagaggagatgcggagaatactctctttcagctattctaatctaccttcgcatctgaaaacttgtctactgtatctatgtgtatatccagaagatagtatgatttctagagataaactgatatggaagtgggtggctgaaggatttgtccaccatgaaaatcaaggaaatagcctgtatttgctcggattaaattacttcaaccagctcattaatagaagtatgatccagccaatatataattatagcggcgaggcatatgcttgccgtgtacatgatatggttctggaccttatctgcaacttgtcatatgaagcaaagtttgtgaatctattggatggcactgggaatagcatgtcttcacagagtaattgtcgccgtttgtcccttcaaaaaagaaatgaagatcatcaagtcaggcctttcacagatatcaagagtatgtcacgagtgaggtcaattactatctttccatctgctattgaagtcatgccatctctttcaaggtttgacgttttacgtgtacttgatctgtcacgatgtaatcttggggagaatagcagcctgcagcttaacctcaaggatgttggacatttaactcacctaaggtaccttggtctagaaggtaccaacatcagtaagctccctgctgagataggaaaactgcagtttttggaggtgttggatcttggaaacaatcgtaatataaaggaattgccgtccacagtttgtaatttcagaagattaatctacctaaatttagttggctgtcaggtggttcctccagttggtttgttgcaaaatctaacagccatagaagtgttgaggggtatcttggtctctctgaacattattgcacaagagcttggcaagttgaaaagtatgagggagcttgagattcgcttcaatgatggtagtttggatttgtatgaaggtttcgtgaagtctctttgcaacttacatcacatagaaagcctaatcattggttgcaattctagagaaacatcatcttttgaagtgatggatctcttgggagaacggtgggtgcctcctgtacatctccgtgaatttgagtcgtccatgcctagccaactctctgcactgcgagggtggataaagagagacccctcccatctctcaaacctctccgacttagtcctgccagtgaaggaagtgcaacaggatgacgtggaaatcattggggggttgctggcccttcgccgtctctggataaagagcaaccaccaaacacaacggctgctagtcatccctgtagatgggttccactgtattgttgactttcagttggactgtggatctgccacgcagatattgtttgagcctggagctttgccgagggcagaatcagttgtgatcagtctgggcgtgcgggtggcgaaagaggatggtaaccgtggcttcgacttgggcctgcaagggaacttgctatcccttcggcggcatgtctttgttcttatctattgtggtggagcgagggttggggaggcaaaggaagcgaaggctgcgctgaggcgtgcccaggaagctcatcccgaccatctccggatttatattgacatgaggccgtgtatagcagaaggtgctcatgatgacgatttgtgtgagggcgaggaggagaactaatttctgatccagagcgactcacattgcatcanatgtgctctcgaggtagcancggcncggggcgttggagttacagctggtggcatcagagatgcttgtttcacaaacagttcgggcgggcgctgaccatgcaaatgtttcgaactttgctggaacttgtgtgatgagcttcttttaaatggcactcagcttgcagaaagaaacatggttttgttttgtaatgaataagcaagggtgttggggtgaattgatccttacaaggatagctttgcttttctttagttgagggccatcgttgctgctctgttttgcatgttgttgttacatgggaggacatgctagtgtattttgtttttaagntgagccgaacaancctgagtatgtattatcagttccgtgttgaatgaaatntgagctcattaaaaaaaaaaaaaaaaaaaaaaaaa

SEQ ID NO: 43 - DNA, 6793 bp, Hordeum vulgare
aactatgtttaaaaaacttccaggaattttttgacttttttttaatttctaaattatttttaaattcaggtgcactggaacatgagactcattgggtatttccggtgttgatttgaggagtaatttaccacctggcaaatgactgcatagacagaggagtaatgcatgatgtggactgaccaaccaactgaggagattcagagaaatgagaggagagtaaatgcagtgaatgatggctggtggacggaccatatacagtgtatgtaattattttgctctgaatccctgtctctctgtgacccactgaataaacacatcagccaaaagcagtactgttcggacttcggagggatcgtggagtagtagtaatttcctctcttgactgttgttcctctgagtcctgtgctccccgcctccactgactgctacctccatctcgtctcagtcctctccttcatttcaagctgtgaaccgaaaacatgcacccagtccggccttgatgtaatgcaggcaaccaatcgacatggagatgtcgatttttagcgtatatatgcttagccagacccaactagatcaaatatgcaaggtacctgaaaacgatgccggtaaccccaaatcgcgtcgtgaaccggagtaatgctagacttacgtaaagatttacatatgtttacgggccgggctgatttggctatgtttgattggattaggtggaggattaggcccacccatcctgaaaatcaggaaggggtcagtattattagtttaatgaaaagggagaattagtacgtaagattttgtasacttttacgtaagtctagcattattgttaaccaccacagtccacgtctctgcgtccgctcatatcaccttgctcgatcgtctcctccacaaacttttctttccggccgtgtgtggatgatagtgtgtactctctagcagttgattgaaggattggactgagtctagtcgacgctagtgacctaaggggacgaagatgcgaggaaggccggtcctgtactctctcgtccatgcatgtcgcgagctgcgtcgtccccatcaccgccaccaccaccgccatggtaggtctccaccttggtcgacctcctccacagacttttcgcaccaattaattccggccagtcggcgacgaccacttcccgtggtgctggtgaatgaatttatgcgtgtgtgtgctatgcttgtcattccagagatatgccagttgcgttctcacggctgagtcattggcacctcaccttctgtctctctcgttaaatttgtatcgatatataagtgcttttgagtacttgcatatataagtgcttttggatctaaaaagttattagttttcatgcttaagtatctgatcaatttgcggtggtagtggcatctttcttgctattctgctctaatgaaatctttcacgtccacacgttcttgttatagatctgctgatttgcttagattataagttcttcttattcttccagatcgattggagcgaccctcacgcctctggtgcgccgtcgctgtgttctgctccgccgtgaagaatcaaggtgggcttggtccagatctagctaagctttaatttcgcagcttgttcaaggcttcacacaatttggattgcgttacagctccctttattcatcaatttacaggcttccagctgattgatacggagatctcgtccctcctgctctcatggatattgtcaccggtgccatttccaacctgattcccaagttgggggagctgctcacggaggagttcaagctgcacaagggtgtcaagaaaaatattgaggacctcgggaaggagcttgagagcatgaacgctgccctcatcaagattggtgaggtgccgagggagcagctcgacagccaagacaagctctgggccgatgaggtcagagagctctcctacgtcattgaggatgtcgtcgacaaattcctcgtacaggttgatggcattcagtctgatgataacaacaacaaatttaaggggctcatgaagaggacgaccgagttgttgaagaaagtcaagcataagcatgggatagctcacgcgatcaaggacatccaagagcaactccaaaaggtggctgataggcgtgacaggaacaaggtatttgttcctcatcctacgagaccaattgctattgacccttgccttcgagctttgtatgctgaagcgacagagctagttggcatatatggaaagagggatcaagacctcatgaggttgctttccatggagggcgatgatgcctctaataagagactgaagaaggtctccattgttggatttggagggttgggcaagaccactcttgctagagcggtatacgagaagattaaaggtgattttgattgtcgggcatttgttccggtcggtcagaaccctgacatgaagaaggttttaagggatatcctcattgatctcggaaatcctcactcagatcttgcgatgctggatgccaatcagcttattaaaaagcttcatgaatttctagagaacaaaaggtatgcatcaatttagaaaaaagtacactattatgtgatgtttgtttcctatgctagtggaacggattagaatatttttttcatcaaggtcacctttactggcataagcagttcacactaaacagtaaaccttataggtgaaaaatttcaggcatgtatatatatatatatatgtttgattctttccggcttaacaaaataattagcaagtacttcttgttgcatttgttccaacggctgaatttattggcaccagtccaagaaatccatctaaatgttttacatttcaccaaagtgtgtgtcatgacagatgtaacaaataataaaccaaaaggagaggaaggaaagaggaagataaatgttacaaaaatttaaatcaaacttatttctacctttctccttacctacccagttgtaaaacacatattatattttaaagagaggcaacatgcgccaaaggctgcccttgaaaattcctaaaatattgtacatttgactcatgaccaaacaaaaagttaaattgtctcttccttatcgcattatatttccatgcatgcctttttctggaaacttactattagcaaaatttagacgaaaggatgatgccacataatttcagtctccagagatttgttagttgccatatattaaattggkgtgccaatctatacctgggccttttttatgtatctacttgatcatttgaacttctgtagttaattgtattctatgaatgatcactcatccaaaaacttgctatttgtgtttcactttgttgagtcttgaatatttattcattttgttcatcatacgattggaggcccataatggatgcttaatgagagtaagattatcgagctccaaacacatgcttcttactagtgtttgaatatatagccttatagatgtatagttcaacccatagattcatatgaccctcagctttctgatgtgtatatataaccttacactgacactgtgaattaatgtaggtatcttgtcataattgatgatatatgggatgaaaaattgtgggaaggcatcaactttgctttctccaataggaataatctaggcagtcgactaatcaccacaacccgcattgtcagtgtctctaattcatgttgctcatcagatggtgattcagtttatcaaatggaaccgctttctgttgatgactctagaatgctcttctccaaaagaatatttcctgatgagaatggatgtataaatgaatttgaacaagtatccagagatattctaaagaaatgtggtggggtaccactagccataattactatagctagtgctttggctggtgaccagaagatgaaaccaaaatgtgagtgggatattctccttcggtcccttggctctggactaacagaagataacagtttagaggagatgcggagaatactctctttcagctattctaatctaccttcgcatctgaaaacttgtctactgtatctatgtgtatatccagaagatagtatgatttctagagataaactgatatggaagtgggtggctgaaggatttgtccaccatgaaaatcaaggaaatagcctgtatttgctcggattaaattacttcaaccagctcattaatagaagtatgatccagccaatatataattatagcggcgaggcatatgcttgccgtgtacatgatatggttctggaccttatctgcaacttgtcatatgaagcaaagtttgtgaatctattggatggcactgggaatagcatgtcttcacagagtaattgtcgccgtttgtcccttcaaaaaagaaatgaagatcatcaagtcaggcctttcacagatatcaagagtatgtcacgagtgaggtcaattactatctttccatctgctattgaagtcatgccatctctttcaaggtttgacgttttacgtgtacttgatctgtcacgatgtaatcttggggagaatagcagcctgcagcttaacctcaaggatgttggacatttaactcacctaaggtaccttggtctagaaggtaccaacatcagtaagctccctgctgagataggaaaactgcagtttttggaggtgttggatcttggaaacaatcgtaatataaaggaattgccgtccacagtttgtaatttcagaagattaatctacctaaatttagttggctgtcaggtggttcctccagttggtttgttgcaaaatctaacagccatagaagtgttgaggggtatcttggtctctctgaacattattgcacaagagcttggcaagttgaaaagtatgagggagcttgagattcgcttcaatgatggtagtttggatttgtatgaaggtttcgtgaagtctctttgcaacttacatcacatagaaagcctaatcattggttgcaattctagagaaacatcatcttttgaagtgatggatctcttgggagaacggtgggtgcctcctgtacatctccgtgaatttgagtcgtccatgcctagccaactctctgcactgcgagggtggataaagagagacccctcccatctctcaaacctctccgacttagtcctgccagtgaaggaagtgcaacaggatgacgtggaaatcattggggggttgctggcccttcgccgtctctggataaagagcaaccaccaaacacaacggctgctagtcatccctgtagatgggttccactgtattgttgactttcagttggactgtggatctgccacgcagatattgtttgagcctggagctttgccgagggcagaatcagttgtgatcagtctgggcgtgcgggtggcgaaagaggatggtaaccgtggcttcgacttgggcctgcaagggaacttgctatcccttcggcggcatgtctttgttcttatctattgtggtggagcgagggttggggaggcaaaggaagcgaaggctgcgctgaggcgtgcccaggaagctcatcccgaccatctccggatttatattgacatgaggccgtgtatagcagaaggtatcgcatgttgcacctaactaattacttgtgcacttacgcatgtgttttttttctcaatgaccgactaaccttattactttctgtgttggttttgatctctaaatctcccaaggctcatgatgacgatttgtgtgagggcgaggaggagaactaatttctgatccagagcgactcacattgcacagatgtgctctcgaggtagcagcggcgcggggcgttggagttacagctggtggcatcagagatgcttgtttcacaaacagttcgggcgggcgctgaccatgcaaatgtttcgaactttgctggaacttgtgtgatgagcttcttttaaatggcactcagcttgcagaaagaaacatggttttgttttgtaatgaataagcaagggtgttggggtgaattgatccttacaaggatagctttgcttttctttagttgagggccatcgttgctgctctgttttgcatgttgttgttacatgggaggacatgctagtgtattttgtttttaagctgagccgaacaaacctgagtatgtattatcagttccgtgttgaatgaaatctgagctcattaattcaataaaaactgtggtttactgttggacttgttacttaaaaactacccacttcgtccggaattattagcatttagagacatccatttgaacctcaggtagttctggacggaggtagtactatttactagttctactaacatgtttgtgtttacatacaaatgaaaagtgtgattcgaactaacaagtacgtacgatttctaaggtgtgcttccaactaacaagcatgtgtaccccaatggcagcaaattatttttgtatgtttgaaaacgttgtcgaaaagccaaataaagcctaaatccaacagtgacaaaaagggccagatatttgtgccgatttaaccgcgtcattctccgtagtttttcatttactccctatatgtattcttatgtgttccagctctgtcatacacatagtgaactcagtggtggtaaaagtcgatcaagggaagcataagcgtcgactagggatgaaaatggagtgaaaactttccgcttttctagagggaaaatgaaaagggggaggaaacatgaaaacaaaaaaaaaggaatttgcaaaatggaagtggaaatggattttttatgctgaaacagaaataaaaacagaacggtgttttccaatgaacgtactcaactagaccctatacacaattgttcagtgaaacttcaatactacgccaagttgctaacat

SEQ ID NO: 44 - DNA, 7153 bp, Hordeum vulgare
aactatgtttaaaaaacttccaggaattttttgacttttttttaatttctaaattatttttaaattcaggtgcactggaacatgagactcattgggtatttccggtgttgatttgaggagtaatttaccacctggcaaatgactgcatagacagaggagtaatgcatgatgtggactgaccaaccaactgaggagattcagagaaatgagaggagagtaaatgcagtgaatgatggctggtggacggaccatatacagtgtatgtaattattttgctctgaatccctgtctctctgtgacccactgaataaacacatcagccaaaagcagtactgttcggacttcggagggatcgtggagtagtagtaatttcctctcttgactgttgttcctctgagtcctgtgctccccgcctccactgactgctacctccatctcgtctcagtcctctccttcatttcaagctgtgaaccgaaaacatgcacccagtccggccttgatgtaatgcaggcaaccaatcgacatggagatgtcgatttttagcgtatatatgcttagccagacccaactagatcaaatatgcaaggtacctgaaaacgatgccggtaaccccaaatcgcgtcgtgaaccggagtaatgctagacttacgtaaagatttacatatgtttacgggccgggctgatttggctatgtttgattggattaggtggaggattaggcccacccatcctgaaaatcaggaaggggtcagtattattagtttaatgaaaagggagaattagtacgtaagattttgtasacttttacgtaagtctagcattattgttaaccaccacagtccacgtctctgcgtccgctcatatcaccttgctcgatcgtctcctccacaaacttttctttccggccgtgtgtggatgatagtgtgtactctctagcagttgattgaaggattggactgagtctagtcgacgctagtgacctaaggggacgaagatgcgaggaaggccggtcctgtactctctcgtccatgcatgtcgcgagctgcgtcgtccccatcaccgccaccaccaccgccatggtaggtctccaccttggtcgacctcctccacagacttttcgcaccaattaattccggccagtcggcgacgaccacttcccgtggtgctggtgaatgaatttatgcgtgtgtgtgctatgcttgtcattccagagatatgccagttgcgttctcacggctgagtcattggcacctcaccttctgtctctctcgttaaatttgtatcgatatataagtgcttttgagtacttgcatatataagtgcttttggatctaaaaagttattagttttcatgcttaagtatctgatcaatttgcggtggtagtggcatctttcttgctattctgctctaatgaaatctttcacgtccacacgttcttgttatagatctgctgatttgcttagattataagttcttcttattcttccagatcgattggagcgaccctcacgcctctggtgcgccgtcgctgtgttctgctccgccgtgaagaatcaaggtgggcttggtccagatctagctaagctttaatttcgcagcttgttcaaggcttcacacaatttggattgcgttacagctccctttattcatcaatttacaggcttccagctgattgatacggagatctcgtccctcctgctctcatggatattgtcaccggtgccatttccaacctgattcccaagttgggggagctgctcacggaggagttcaagctgcacaagggtgtcaagaaaaatattgaggacctcgggaaggagcttgagagcatgaacgctgccctcatcaagattggtgaggtgccgagggagcagctcgacagccaagacaagctctgggccgatgaggtcagagagctctcctacgtcattgaggatgtcgtcgacaaattcctcgtacaggttgatggcattcagtctgatgataacaacaacaaatttaaggggctcatgaagaggacgaccgagttgttgaagaaagtcaagcataagcatgggatagctcacgcgatcaaggacatccaagagcaactccaaaaggtggctgataggcgtgacaggaacaaggtatttgttcctcatcctacgagaccaattgctattgacccttgccttcgagctttgtatgctgaagcgacagagctagttggcatatatggaaagagggatcaagacctcatgaggttgctttccatggagggcgatgatgcctctaataagagactgaagaaggtctccattgttggatttggagggttgggcaagaccactcttgctagagcggtatacgagaagattaaaggtgattttgattgtcgggcatttgttccggtcggtcagaaccctgacatgaagaaggttttaagggatatcctcattgatctcggaaatcctcactcagatcttgcgatgctggatgccaatcagcttattaaaaagcttcatgaatttctagagaacaaaaggtatgcatcaatttagaaaaaagtacactattatgtgatgtttgtttcctatgctagtggaacggattagaatatttttttcatcaaggtcacctttactggcataagcagttcacactaaacagtaaaccttataggtgaaaaatttcaggcatgtatatatatatatatatgtttgattctttccggcttaacaaaataattagcaagtacttcttgttgcatttgttccaacggctgaatttattggcaccagtccaagaaatccatctaaatgttttacatttcaccaaagtgtgtgtcatgacagatgtaacaaataataaaccaaaaggagaggaaggaaagaggaagataaatgttacaaaaatttaaatcaaacttatttctacctttctccttacctacccagttgtaaaacacatattatattttaaagagaggcaacatgcgccaaaggctgcccttgaaaattcctaaaatattgtacatttgactcatgaccaaacaaaaagttaaattgtctcttccttatcgcattatatttccatgcatgcctttttctggaaacttactattagcaaaatttagacgaaaggatgatgccacataatttcagtctccagagatttgttagttgccatatattaaattggtgtgccaatctatacctgggccttttttatgtatctacttgatcatttgaacttctgtagttaattgtattctatgaatgatcactcatccaaaaacttgctatttgtgtttcactttgttgagtcttgaatatttattcattttgttcatcatacgattggaggcccataatggatgcttaatgagagtaagattatcgagctccaaacacatgcttcttactagtgtttgaatatatagccttatagatgtatagttcaacccatagattcatatgaccctcagctttctgatgtgtatatataaccttacactgacactgtgaattaatgtaggtatcttgtcataattgatgatatatgggatgaaaaattgtgggaaggcatcaactttgctttctccaataggaataatctaggcagtcgactaatcaccacaacccgcattgtcagtgtctctaattcatgttgctcatcagatggtgattcagtttatcaaatggaaccgctttctgttgatgactctagaatgctcttctccaaaagaatatttcctgatgagaatggatgtataaatgaatttgaacaagtatccagagatattctaaagaaatgtggtggggtaccactagccataattactatagctagtgctttggctggtgaccagaagatgaaaccaaaatgtgagtgggatattctccttcggtcccttggctctggactaacagaagataacagtttagaggagatgcggagaatactctctttcagctattctaatctaccttcgcatctgaaaacttgtctactgtatctatgtgtatatccagaagatagtatgatttctagagataaactgatatggaagtgggtggctgaaggatttgtccaccatgaaaatcaaggaaatagcctgtatttgctcggattaaattacttcaaccagctcattaatagaagtatgatccagccaatatataattatagcggcgaggcatatgcttgccgtgtacatgatatggttctggaccttatctgcaacttgtcatatgaagcaaagtttgtgaatctattggatggcactgggaatagcatgtcttcacagagtaattgtcgccgtttgtcccttcaaaaaagaaatgaagatcatcaagtcaggcctttcacagatatcaagagtatgtcacgagtgaggtcaattactatctttccatctgctattgaagtcatgccatctctttcaaggtttgacgttttacgtgtacttgatctgtcacgatgtaatcttggggagaatagcagcctgcagcttaacctcaaggatgttggacatttaactcacctaaggtaccttggtctagaaggtaccaacatcagtaagctccctgctgagataggaaaactgcagtttttggaggtgttggatcttggaaacaatcgtaatataaaggaattgccgtccacagtttgtaatttcagaagattaatctacctaaatttagttggctgtcaggtggttcctccagttggtttgttgcaaaatctaacagccatagaagtgttgaggggtatcttggtctctctgaacattattgcacaagagcttggcaagttgaaaagtatgagggagcttgagattcgcttcaatgatggtagtttggatttgtatgaaggtttcgtgaagtctctttgcaacttacatcacatagaaagcctaatcattggttgcaattctagagaaacatcatcttttgaagtgatggatctcttgggagaacggtgggtgcctcctgtacatctccgtgaatttgagtcgtccatgcctagccaactctctgcactgcgagggtggataaagagagacccctcccatctctcaaacctctccgacttagtcctgccagtgaaggaagtgcaacaggatgacgtggaaatcattggggggttgctggcccttcgccgtctctggataaagagcaaccaccaaacacaacggctgctagtcatccctgtagatgggttccactgtattgttgactttcagttggactgtggatctgccacgcagatattgtttgagcctggagctttgccgagggcagaatcagttgtgatcagtctgggcgtgcgggtggcgaaagaggatggtaaccgtggcttcgacttgggcctgcaagggaacttgctatcccttcggcggcatgtctttgttcttatctattgtggtggagcgagggttggggaggcaaaggaagcgaaggctgcgctgaggcgtgcccaggaagctcatcccgaccatctccggatttatattgacatgaggccgtgtatagcagaaggtatcgcatgttgcacctaactaattacttgtgcacttacgcatgtgttttttttctcaatgaccgactaaccttattactttctgtgttggttttgatctctaaatctcccaaggtgctcatgatgacgatttgtgtgagggcgaggaggagaactaatttctgatccagagcgactcacattgcatcagatgtgctctcgaggtatgtagcagataagaaacagattaaggtatttacaaaaattgcttagacataagtatctgatcagaaaagtggacttggcagtgtagtgtgaaacttgcctagtcacttttttggcaaggggtgatgaaagataagaattattttatgcaaattgatagaaggatagtcagtaatggggaattggggatatgactagattttcagaagttatatgtacaaggagtgttgttttaccgaaaaggctttcatcccggtttatatataaagcaaaccaccagatcaagagtacaagcataagaccaaaccagacacgcatacacatacccaagatagaacgacgtcaaatacgggggttctgctgagggcacagctcaacaagccctaaaaaacaaaataaggcggagggaccgcaatagaagcaactaatctggctctggaggtggtggcggaaccaagcggaaggccatcatccgcagatctgcgatgatggagtcgatggcgtcctgatccgggaggggtgcttgttttaggtgacaagtaccccaggttatacagtttatctgtagctagagtgctacacgctaatttcactgctctaaaatgtaaagggatcttgtatggtgaaactgctgagctacgacatcgattgcttgctgatcgtgaaggctttggtcttgaggaatgagcaagattcatgtagatgacgccgcaaaataaatatatacgtaatggctttcaaagaataatggggtattttctgtgcatcctatatttatagctttgaatgctcaacaagtgaaatgaccataaagaaaattttggcatgtaaaaggtccacttacgatcatagttttttatagttagcattcagaaatagtatcggcagagttaatctgaatcgtcgaggaatggtaattgcttgaaaaatgttttcgctgaggatatttgatgttttattggtctgtctaacaaagaatagaaatgcacgatatgtaggtagcagcggcgcggggcgttggagttacagctggtggcatcagagatgcttgtttcacaaacagttcgggcgggcgctgaccatgcaaatgtttcgaactttgctggaacttgtgtgatgagcttcttttaaatggcactcagcttgcagaaagaaacatggttttgttttgtaatgaataagcaagggtgttggggtgaattgatccttacaaggatagctttgcttttctttagttgagggccatcgttgctgctctgttttgcatgttgttgttacatgggaggacatgctagtgtattttgtttttaagctgagccgaacaaacctgagtatgtattatcagttccgtgttgaatgaaatctgagctcattaattcaataaaaactgtggtttactgttggacttgttacttaaaaactacc

SEQ ID NO: 45 - DNA, 3434 bp, Artificial Sequence
  Mla12 cDNA
ctgacacccgtggatctaaaaagttattagttttcatgcttaagtatctgatcaatttgcggtgatcgattggagcgatcctcacgcctctggtgcgccgtcgctgtgttctgctccgccgtgaagaatcaaggcttccagctgattatagggctgattgatacggatatctcgtcctccagctctcatggatattgtcaccggtgccatttccaacctgattcccaagttgggggagctactcacggaggagttcaagctgcacaagggtgtcaagaaaaatattgaggacctcgggaaggagcttgagagcatgaacgctgccctcatcaagattggtgaggtgccgagggagcagctcgacagccaagacaagctctgggccgatgaggtcagagagctctcctacgtcattgaggatgtcgtcgacaagttcctcgtacaggttgatggcattaagtctgatgataacaacaacaaatctaaggggctcatgaagaggactaccgagttgttgaagaaagtcaagcataagcatgggatagctcacgcgatcaaggacatccaagagcaactccaaaaggtggctgataggcgtgacaggaacaaggtatttgttcctcatcctacgagaacaattgctattgacccttgccttcgagctttgtatgctgaagcgacagagctagttggcatatatggaaagagggatcaaggcctcatgaggttgctttccatggagggcgatgatgcctctaataagagactgaagaaggtctccattgttggatttggagggttgggcaagaccactcttgctagagcggtatacgagaagattaaaggtgatttcgattgtcgggcatttgttccggtcggtcagaaccctgacatgaagaaggttttaagggatatcctcattgatctcggaaatcctcactcagatcttgcgatgctggatgccaatcagcttattaaaaagcttcatgaatttctagagaacaaaaggtatcttgtcataattgatgatatatgggatgaaaaattgtgggaaggcatcaactttgctttctccaataggaataatctaggcagtcggctaatcaccacaacccgcattgtcagtgtctctaattcatgttgctcatcagatggtgattcagtttatcaaatggaaccgctttctgttgatgactccagaatgctcttctacaaaagaatatttcctgatgagaatgcatgtataaatgaatttgaacaagtatccagagatattctaaagaaatgtggtggggtaccactagccataattactatagctagtgctttggctggtgaccagaagatgaaaccaaaatgtgagtgggatattctccttcggtcccttggctctggactaacagaagataacagtttagaggagatgcggagaatactctctttcagctattctaatctaccttcgaatctgaaaacttgtctactgtatctatgtgtatatccagaagatagtatgatttctagagataaactgatatggaagtgggtggccgaaggatttgtccaccatgaaaatcaaggaaatagcctgtatttgctcggattaaattacttcaaccagctcattaatagaagtatgatccagccaatatataattatagcggcgaggcatatgcttgccgtgtacatgatatggttctggaccttatctgcaacttgtcacgtgaagcaaagtttgtgaatctattggatggcactgggaatagcatgtcttcacagagtaattgtcgtcgtttgtcccttcagaaaagaaatgaagatcatcaagccaggcctctcatagatatcaagagtatgtcacgagtgaggtcaattactatctttccacctgctattgaagtcatgccatctctttcaaggtttgaggttttatgtgtacttgatttgtcgaaatgtaatcttggggaggatagcagcctgcaacttaacctcaaggatgttggacaattaattcagctaaggtaccttggtctagaatgtaccaatataagtaagctcccgactgagataggaaaactgcagtttttggaggtgttggatcttggaaacaatcctaatctaaaagaattgccgtccactattcgtaatttcagaagattaatctacctaaatttagttggctgtcaggtgattcctccagtgggtgtgttgcaaaatctgacatccatagaagtattgaggggtatcttggtctatctgaacattattgcacaagagcttggcaacctggaaagggtgagagatcttgagattcgcttcaatgatggtagtttggatttgtatgaaggtttggtgaattctctgtgcaacctacatcacatcgaaagtctaaatattcgttgcaatcccggagaaacatcatcttttgaactgatggatctcttggaagaacgttgggtgccgcctgtacatctccgtgaatttaagtcattcatgcccagccaactctctgcactgcgagggtggatacagagagacccctcccatctctcgaacctctccgagttaaccctctggccagtgaaggacgtgcagcaggatgacgtggaaatcattggggggttgttgtcccttcgtcgtctctggatagtaaagagcatccaccaaacgcaacggctgctagtcatccgtgcagatgggttccgctctatggttgaatttcgtttggattgtggatctgccacgcagatattgtttgagccaggagctttgccgagggcggaatcagttgtgatcagtctgggcgtgcgggtggcgaaagaggatggtaaccgtggcttccacttgggcctgcaggaagcaaaggatgtctcccttcggtgggatgtctttgttcttctctattgtggtggagcgagggttggggaggcaaaggaagcggaggctgcggtgaggcgtgccctggaagctcatcccagacatcctcggatttatattgacatgaggccggatatacaggaaggtgctcatgatgacgatttgtgtgagaacgaggacgagggtgagaactgatttttggcgcagaaggattcacactgcatcaggactgctctcggtagcaggggtgcggggttactgctggaggcatcggacatgcatgtttcacaaacattttggatgggtgccgaccgggcgaagaattgaaggatggaaagttttcgaacttttctgaaagttgggtgatgagcttcttttaaatggcagtcgcttgccgaaagagatactgcttggttctgtaatgaataagtaacggtgttggatcgaattgatccttacaagtatatctttgcttttcttctgcctgcaaatcgctccccatttcacccaattgtaaatatgctaactccagcaaagaccttgatgaatctttggg

SEQ ID NO: 46 - protein, 961 aa, Hordeum vulgare
MDIVTGAISNLIPKLGELLTEEFKLHKGVKKNIEDLGKELESMNAALIKIGEVPREQLDSQDKLWADEVRELSYVIEDVVDKFLVQVDGIKSDDNNNKSKGLMKRTTELLKKVKHKHGIAHAIKDIQEQLQKVADRRDRNKVFVPHPTRTIAIDPCLRALYAEATELVGIYGKRDQGLMRLLSMEGDDASNKRLKKVSIVGFGGLGKTTLARAVYEKIKGDFDCRAFVPVGQNPDMKKVLRDILIDLGNPHSDLAMLDANQLIKKLHEFLENKRYLVIIDDIWDEKLWEGINFAFSNRNNLGSRLITTTRIVSVSNSCCSSDGDSVYQMEPLSVDDSRMLFYKRIFPDENACINEFEQVSRDILKKCGGVPLAIITIASALAGDQKMKPKCEWDILLRSLGSGLTEDNSLEEMRRILSFSYSNLPSNLKTCLLYLCVYPEDSMISRDKLIWKWVAEGFVHHENQGNSLYLLGLNYFNQLINRSMIQPIYNYSGEAYACRVHDMVLDLICNLSREAKFVNLLDGTGNSMSSQSNCRRLSLQKRNEDHQARPLIDIKSMSRVRSITIFPPAIEVMPSLSRFEVLCVLDLSKCNLGEDSSLQLNLKDVGQLIQLRYLGLECTNISKLPTEIGKLQFLEVLDLGNNPNLKELPSTIRNFRRLIYLNLVGCQVIPPVGVLQNLTSIEVLRGILVYLNIIAQELGNLERVRDLEIRFNDGSLDLYEGLVNSLCNLHHIESLNIRCNPGETSSFELMDLLEERWVPPVHLREFKSFMPSQLSALRGWIQRDPSHLSNLSELTLWPVKDVQQDDVEIIGGLLSLRRLWIVKSIHQTQRLLVIRADGFRSMVEFRLDCGSATQILFEPGALPRAESVVISLGVRVAKEDGNRGFHLGLQEAKDVSLRWDVFVLLYCGGARVGEAKEAEAAVRRALEAHPRHPRIYIDMRPDIQEGAHDDDLCENEDEGEN

SEQ ID NO: 47 - protein, 961 aa, Hordeum vulgare
MDIVTGAISNLIPKLGELLTEEFKLHKGVKKNIEDLGKELESMNAALIKIGEVPREQLDSQDKLWADEVRELSYVIEDVVDKFLVQVDGIKSDDNNNKSKGLMKRTTELLKKVKHKHGIAHAIKDIQEQLQKVADRRDRNKVFVPHPTRTIAIDPCLRALYAEATELVGIYGKRDQGLMRLLSMEGDDASNKRLKKVSIVGFGGLGKTTLARAVYEKIKGDFDCRAFVPVGQNPDMKKVLRDILIDLGNPHSDLAMLDANQLIKKLHEFLENKRYLVIIDDIWDEKLWEGINFAFSNRNNLGSRLITTTRIVSVSNSCCSSDGDSVYQMEPLSVDDSRMLFYKRIFPDENACINEFEQVSRDILKKCGGVPLAIITIASALAGDQKMKPKCEWDILLRSLGSGLTEDNSLEEMRRILSFSYSNLPSNLKTCLLYLCVYPEDSMISRDKLIWKWVAEGFVHHENQGNSLYLLGLNYFNQLINRSMIQPIYNYSGEAYACRVHDMVLDLICNLSREAKFVNLLDGTGNSMSSQSNCRRLSLQKRNEDHQARPLIDIKSMSRVRSITIFPPAIEVMPSLSRFEVLCVLDLSKCNLGEDSSLQLNLKDVGQLIQLRYLGLECTNISKLPTEIGKRQFLEVLDLGNNPNLKELPSTIRNFRRLIYLNLVGCQVIPPVGVLQNLTSIEVLRGILVYLNIIAQELGNLERVRDLEIRFNDGSLDLYEGLVNSLCNLHHIESLNIRCNPGETSSFELMDLLEERWVPPVHLREFKSFMPSQLSALRGWIQRDPSHLSNLSELTLWPVKDVQQDDVEIIGGLLSLRRLWIVKSIHQTQRLLVIRADGFRSMVEFRLDCGSATQILFEPGALPRAESVVISLGVRVAKEDGNRGFHLGLQEAKDVSLRWDVFVLLYCGGARVGEAKEAEAAVRRALEAHPRHPRIYIDMRPDIQEGAHDDDLCENEDEGEN

SEQ ID NO: 48 - protein, 961 aa, Hordeum vulgare
MDIVTGAISNLIPKLGELLTEEFKLHKGVKKNIEDLGKELESMNAALIKIGEVPREQLDSQDKLWADEVRELSYVIEDVVDKFLVQVDGIKSDDNNNKSKGLMKRTTELLKKVKHKHGIAHAIKDIQEQLQKVADRRDRNKVFVPHPTRTIAIDPCLRALYAEATELVGIYGKRDQGLMRLLSMEGDDASNKRLKKVSIVGFGGLGKTTLARAVYEKIKGDFDCRAFVPVGQNPDMKKVLRDILIDLGNPHSDLAMLDANQLIKKLHEFLENKRYLVIIDDIWDEKLWEGINFAFSNRNNLGSRLITTTRIVSVSNSCCSSDGDSVYQMEPLSVDDSRMLFYKRIFPDENACINEFEQVSRDILKKCGGVPLAIITIASALAGDQKMKPKCEWDILLRSLGSGLTEDNSLEEMRRILSFSYSNLPSNLKTCLLYLCVYPEDSMISRDKLIWKWVAEGFVHHENQGNSLYLLGLNYFNQLINRSMIQPIYNYSGEAYACRVHDMVLDLICNLSREAKFVNLLDGTGNSMSSQSNCRRLSLQKRNEDHQARPLIDIKSMSRVRSITIFPPAIEVMPSLSRFEVLCVLDLSKCNLGEDSSLQLNLKDVGQLIQLRYLGLECTNISKLPTEIGKLQFLEVLDLGNNPNLKELPSTIRNFRRLIYLNLVGCQVIPPVGVLQNLTSIEVLRGILVYLNIIAQELGNLERVRDLEIRFNDGSLDLYEGLVNSLCNLHHIESLNIRCNPGETSSFELMDLLEERWVPPVHLREFKSFMPSQLSALRGWIQRDPSHLSNLSELTLWPVKDVQQDDVEIIGGLLSLRRLWIVKSIHQTQRLLVIRADGFRSMVEFRLDCGSATQILFEPGALPRAESVVISLGVRVAKEDGNRGFHLGLQEAKDVSLRWDVFVLLYCGGARVGEAMEAEAAVRRALEAHPRHPRIYIDMRPDIQEGAHDDDLCENEDEGEN

SEQ ID NO: 49 - DNA, 7900 bp, Hordeum vulgare
gcgtcgcgtggctctggcgactcaagtgcaaaacctagcgtcacgactagccctctccccagcacctcaccgtctcgccgtcggcagaggttcccgccgtcaaagctcgcgtagataccagggacgacggcggagggccgtctctctccgggtggcgatgcgtggcgcagggtgggcgacgcatcttgccatggtcggcggcggtctgctccgtgcgtgctgatgtgtggtggtggatgcgaggcggcggtgggcgcccgtccagtcgcgccgcggtggctagaggactgggtggtccggatcttgtcctgtgagccagtctggcgtggtggcgggtctgcaggtggaggaaggaagaagtggtggcgcggtgcagaggtggtgggcggcgtggtgtgggtgcacgggctacagtggcacctcggcggccggagtacctgatggtctggatctagtccagcgtgccggtccggcgcggcgacgggcctgcaggtggcgcgggaaagaagtggcggcgtggagcggaggtggtgggcggcatggtgtgggtacacaggcctccgtcaaggtgtgcaaccgacatgggttctggcgggtgctcgtcgcccggcaggggcgttcgaggggcggaggagctatcgcggatttggtgatgagctcttgggcggcggcttctgccgcgaggggcacagccggttggtgtgtgtgcggtatggcatcgcgtggacgcccgtggggatgcatagatcgaaggcccatggcagcgaggatgtgcactccagcggcgtatgatgcggatctggtttccggacgtcggtggtctccacgaggtgcaggtgtatgcgatggatagtggtgcttcttcgtcaatgggcagcttcgatggtggtgatgtttgttgggtttgccgacgccaatgcgggatgatgtgtggctaaaggtggtgtggagcttggtgaaaaccatgtcttggcctcgttccatgaccatgtatggaagtttgctccaatctgatgtgccacttgataataactagaagatttgaaagatgaagacacctcttaagaataaaatatttgcatggtatctttatcgcggagcaattcttactaaagataaccttattaagaggaattgacatgtagatacacaatgtgttttttgtcaacatgatgagacaataaaagatttgttcttccaatgcaaattcgctcgttctatatggccagtcatctaaatagcttttggcttgtatcctccttgtagtgttgcttatacatttggcaactggttatatgggattgaacataggtttagatgtcttctcagggtcgaagcgcttgccgttatttggtcgctttggctacgtagaaatgataaaaaaattaatggtaaaggtactttattatgcaggttatctacaaatgtaccgggacgctttgcttataatcgtctctacaacatctgcagaatcgggacctatttataaaggtatgtacatgattggaggctacgacgagggatatttttatccaatatggatgacagtatgatcttaggattggcctccctatggcttaggcgttatacagactttacatgtttctttgtattttgcatttttttatatgagaggacttcgtttggctgtgtgcacacacagttatgcagaggccgggtataatgcttaaatcttttaagtaataaagctgacttatcaaaaaacaaacacgcgcaaccaaaaaacatgcacccagtccggccttgatgtaatgcaggcaaccaatcgacatggagatgtcaatttttagcgtgtatatgcttagccagatccaactagatcaaatatgcaaggtgcctgaaaacgatgcaggcaatcccaaatcacgtcgttaaccggagtaatgctagacttacgtaaagatttacgtatgtttacgggctgggctgatttggctatgtttgattggattaggtggaggattaggcccacccatactgaaaatcaggaaggggtcagtattagattagattaatgaaaagggagagttagtacgtaagattttgtagacttttatgtaagtctagcattattgttaaccaccacagtccatgtctctgcgtccgctcatatcaccttgctcgatcgtctcctccacaaacttttctttccggccgtgtgtggatgatatatagtgtgtactctgtagcagttgattgaaggattggaccgagtcgacgctagtgacctaaggggacgaagatgcgaggaaggccggtcctgtactctctcgtccatgcatgtcgcgagctgcgtcgtccccatcaccgccaccaccaccgccatggtaggtctccaccttggccgacctcctccacagacttttcgcacaaattaattccggccagtcggagacgaccacttcccgtggtgcttggtgaatgaatttatgcgtgtgcgtgctgtgcttgtcattccagagatatgccagttgcgttctcacggctgagtcatcggcaccttgcatagtagttcgctctgccagagacctttgacacccgtggatctaaaaagatattagttttcatgcttaagtatctgatcaatttgcggtggtagtggcatctttcttggtattctgctctaatgaaatctttcgcgtccacacgttcttgttatagatctgctgatttgcttagactataagttcttcttattcttccagatcgattgaagcgaccctcacgcctctggtgcgccgtcgctgtgttctgctccgccgtgaagaatcaaggtgggcttggtccagatctagcaaagctttaatttggcagcttgttcaaggcttcacacaatttggattgcgttacagctccctttattcatcaatttacaggcttccagctgattgatacggagatctcgtcctcctgctctcatggatattgtcaccggtgccatttccaacctgattcccaagttgggggagctgctcacggaggagttcaagctgcacaagggtgtcaagaaaaatattgaggacctcgggaaggagcttgagagcatgaacgctgccctcatcaagattggtgaggtgccgagggagcagctcgacagccaagacaagctctgggccgatgaagtcagagagctctcctacgtcattgaggatgtcgtcgacaagttcctcgtacaggttgatggcattcagtttgatgataacaacaacaaatttaaggggttcatgaagaggacgaccgagttgttgaagaaagtcaagcataagcatgggatagctcacgcgatcaaggacatccaagagcaactccaaaaggtggctgataggcgtgacaggaacaaggtatttgttcctcatcctacgagaacaattgctattgacccttgccttcgagctttgtatgctgaagcgacagagctagttggcatatatggaaagagggatcaagacctcatgaggttgctttccatggagggcgatgatgcctctaataagagactgaagaaggtctccattgttggatttggagggttgggcaagaccactcttgctagagcggtatacgagaagattaaaggtgatttcgattgtcgggcttttgttccggtcggtcagaaccctcacatgaagaaggttttaagggatatcctcattgatctcggaaatcctcactcagatcttgcgatgctggatgccaatcagcttattaaaaaacttcgtgaatttctagagaacaaaaggtatgcatcaatttagaaaaaagtacactattatgtgatgtttgtttcctatgctagtggaacggattagaatttttttttcattaaggtcacctttactggcataagcagttcacactaaacggtaaaccttataggtgaaaattttcaggcatatatatatatatatatatatatatatgtttgattctttccggcttaacaaaataattagcaagtacttcttgttgcatttgttccaacggctgaatttattggcatcggtccaagaaatccatctaaatgttttacatttcaccaaagtgtgtgtcatgacagatgtaacaaataataaaccaaaaggagaggaaggaaagaggaagataaatgttacaaaaatttaaatcaaacttatttctacctttctccttacctacccagtttaaaaacacatattatattttaaagagaggcaacatgcgccaaaggctacccttgaaaattcctaaaatattgtacatttgactgatgaccaaacaaaaagttaaattgtctcttccttatcacattatatttccatgcatgcctttttctggaaacttactatcagcaaaatttagatgaaaggataatgccacataatttcagtctccaagagatttgttagttgtcatatattaaattggtgggccaatctattcctgggtctttttatgtatctacttgaccatttgaacttctgtagttaattgtattctatgaatgatcactcatccaaaaacttgttatttgtgttttactctgttgaatcttgaatatttattcattttgttcatcatacgattggaggcccataatagatgcttaatgagagtaagattatcgatctccaaacacatgcttcttactagtgttgaatatatacccttttagatgtatagttcaacccatagattcatatgaccctcagctttctgatgtgtatgtatgaccttacactgacactctgaactaatgtaggtatcttgtcataattgatgatatatgggatgaaaaattatgggaaggcatcaactttgctttctccaataggaataatctaggcagtcggctaatcaccacaacccgcattgtcagtgtctctaattcatgttgctcatcacatggtgattcggtttatcaaatggaaccactttctgttgatgactccagaatactcttctggaaaagaatatttccagatgagaatggatgtctaaatgaatttgaacaagtgtcgagagatattctaaagaaatgtggtggggtaccactagccataattaccatagctagtgctttggccggtgaccagaagatgaaaccaaagtgtgagtgggatattctccttcagtcccttggctctggactaacagaagataacagtttagaggagatgcggagaatactctctttcagctattctaatctaccttctcatctgaaaacttgtctactgtatctatgtatatatccagaagatagcaagattcatagagatgaactgatatggaagtgggtggccgaaggatttgtccaccatgaaaaccaaggaaatagcttgtatttgctcggattaaattacttcaaccagctcattaatagaagtatgatccagcccatatatggttttaatgacgaggtatatgtatgtcgtgtacatgatatggttctggaccttatctgcaacttgtcacgtgaagcaaaatttgtgaatctattggatggcagtgggaatagcatgtcttcacagggtaattgtcgccgtctgtcccttcaaaaaagaaatgaagatcatcaagccaaacctatcacagatatcaagagtatgtcacgagtgaggtcaattactatctttccacctgctattgaagtcatgccatctctttcaaggtttgacgttttacgtgtacttgatctgtcacgatgtaatcttggggagaatagcagcctgcagcttaacctgaaggatgttggacatttaactcacctaaggtaccttggtctagaaggtaccaacatcagtaagctccctgctgagataggaaaactgcagtttttggaggtgttggatcttggaaacaatcataatctaaaggaattgccgtccactgtttgtaatttcagaagattaatctacctaaatttatttgggtgtccggtggttcctccagttggtgtgttgcaaaatctgacatccatagaagtgttgagggggatcttggtctctgtgaacattattgcacaagagcttggcaacctggaaaggctgagggtgcttgatatttgcttcagggatggtagtttggatttgtataaagatttcgtgaagtctctgtgcaacctacatcacatcgaaagtctacgtattgagtgcaattccagagaaacatcatcttttgaactggtggatctcttgggagaacgctgggtgcctcctgtacatttccgtgaatttgtgtcatccatgcctagccaactctctgcactgcgagggtggataaagagagacccctcccatctctcgaacctctccgagttaatcctctcgtcagtgaaggacgtgcagcaggatgacgtggaaatcattggggggttgttgtgccttcgtcgtctctttataataacgagcaccgaccaaacgcaacggctgctagtcatccgtgcagatgggttccgctgtacggttgactttcgattggattgtggatctgccacgcagatattgtttgaaccaggagctttgccaagggcggtaagagtttggttcagccttggcgtgcgggtgacgaaagaggatggtaaccgtggcttcgacttgggcctgcaggggaacctgttctcccttcgagagtttgtctctgtttatatgtattgtggtggagcgagggttggggaggcaaaggaagcggaggctgcggtgaggcgtgccctggaagctcatcccagccatccccggatttatattcagatgaggccgcatatagcaaaaggtacgcatcctgcacctaactaattacttgtgcacttacacatgtgtttttttctcaatgacggactgaccttattactttctgcatggattttgatctctaaatctcccaaggtgctcatgatgacgatttgtgtgaggacgaggaggagaactgatttctgatccagagcgactcacattgcatcagatgtgctctgaggtatgtagcagatattgacgacgacatcggttttagatatcgggagtaagagcgctccatattatcgaatttgtaggtgcgagtggtggcttcgggtggattgatgtatgttcttatcatacctttgtggaataattaataaaaataaccgcaggcatcgattgatgcggaggccgcgcgtttaaccttcttttctaaaaaagaaaattctctttgatagtgtgctgtgaccaagatattcaaagaaaggacggagacggaccacatacagttgctttggactctatatcttgtagatgttaatcatgttgctcatgtaggattaggaaagaaagccaaaccgagtcctagtagtattagaattcctagtcctagtctatttccatagtcccttgtggacgtgtataaaagacaccctaggggtgttgattgtaacaccagaaaaacgaaaagcaatacaaagcaaaggctcgacacgggcctttagccatcaaattcattgatcagttttattcgtgtcgctagttacgcaagttccgtcaagtagccggccgaagcaagaatcgcgtaggcacgcctgtacggctgcatacgcacgttcaaaagccaacaattggtatctagagcctcgacgatctacgatctacgatgacggacagcgacgctgagtcggtcaagtccggcagcggcggtgccaagacgaacaagaacggcgacaaggtgaagaagggcgtcaagtcagcaaccagtggtggaggaacgagcggcgctaacgtccaggcgcatcgcaacatccccatccagtacccgatgctcaccgacgccaactacggcgtgtgggcggtgaagatgaagattattcttcgatcccttcgagtgtgggaggccatcacggacgacgacgtcgacgaggagcgcgacgaaggtg

SEQ ID NO: 50 - DNA, 7800 bp, Hordeum vulgare
gagttaggtgggctctctctctctctctcgtgggctcatattttttctccttttctctatggatggatggagggatgtgggggtggagatctagatggatagatggatggatatgcgccacgtcatcgatccgtggaacaaatgtttccaccaataggaatcaagctcatgcaattgtgttcttctttttatttttcttctaattttttctaccctcttgaccgtcgaaggagggaaagtactaactgcatccacatcggtgaaggattcggcggcctctggcctcgtctccatcgaagaagcactgaggtgaaggccaccgaaaccctacccacaatgtttccacgaatgagaagtaacctattgtgtggctgtttccaccctcaagtgaagacgatggtgtcgttcatgaagggggtcacactccggagccacgtggcgggcgtctaaagctattttgccatttaaagagttttctgccgaatcaaaaggaaatcagatggaatcgggaggaaatcggtcaaacaattttgcacaagggtgcatcttggaatggcaaacaatgttgctaaagggagttttcattttctttggacgtaaaattcattttccatttgtggccttgtcgacacctacacaagacacataggcgaaattgttgagttttcttcccagccacctctatttaagcatatgaccttcttaattttgtcatgtatgacctacaatgcgggcataaatagagctacacatgctagtgaccattttggacaatgggattatgaccttcttgatatatagagatggtcaaaaatttatacggtttggacccttttagacacctcatgacctttttgtagattttagtcatagatctatgaccaataaaaccgccatcaatattttttgtcataaacgagcttatatcttgtagtggtaatcgaaaccagcgttcggggcttgatcgatgtcgatgacgttggcggccaacaagttcacaaaatcggcagcggcattgttcgaaccaccgctacattgagtgacaacacatctcgaactaccacaatggagaaaaatcgaagcaaaaataagtgagcgaagatgcataccttccggccctttcatcgaacacctcgcttgcggtggaagcgcgacgtagaaaggcatcgtcaaggaacgtctcgcccgggggatggagtggacgaatgttttttcgcaggaactttcttccacttcacgctcaaaaccttgctcatcttccccgacgacggccgggcagatcgcgtagcagcggcggtgcccggcgtgccttcccttctagcggggccagccggaatgccgccctgcatgacaacatttccttggcgcttgcgggcgggcgcgttggtgactagggtgacggcggggggcgacatggccagcaacgagattcgcccgacaggattgcgcgcgcgcgacctccacatagacgagatccgccggctggaaagcttccatggtggccaagaatggcggggaagaaggtccggtgcacaccgtgggcggggcaatggctgccatggaggggggagcagggaacgaccgcgcggaaattcccctcgtaccaaatctcattgcggataaggggcgagcttgggtcggcctcccaccccgtgaatctgaaggttgaggacgaacttttgccgcgtcccgtaaaaaaaaaattacgagtcgggcgcgtttgcggggtctagtgtgacagcattttctgtcccgacccatattttagaggttattttacgggccgggcattattacggaagagtgagaaaccatatacagtagtctgtaattattttctctgaatccctgagtgtctatgacccaccgaatgaacacatgagcccaaagcactaccgtggggcttctttccacgcccacccaccgaaagcattactccctttcttctttctttacgttctcccacattcaacgcatcgacccactgaacaaaacacatgagccaagagcattgcgtactattacgaggagcatcctatacttctgaaaggccagcattatgctgattaccccagtatagtgctttgatttagcaactctagttgattactagtctctgacctacagaagactatatacatcaacttaagttctatttttagtaatagtagtactcaaaggcaattaatagtagtactcaattcttttaccagttgcaaagaggaaggctaatgagtagagttactagtgtcattaactgataaatgcatgaaggagagagaggtggtaaatgcctggcaggtgcatgagggagaaatgagaaatatgaccatgtacagtactttctaattattgtctctgtatgacccactgaataattttataatgagccaaaagctagtattcagaggcatcgtgggggctctgtatctcttgagtcttgacctgctgttgttggtcttcctctgctcttcacaaccgagtgctccctttctctcatctcagtcctcctctccattgcgctgtcaacctccgagctcgcccctgccaactgtggaggtatatttctttccagccgaagtacccttggccttggatctaaaaacttgcgtctcatgcttcactgcttgagctagaggcctggtaattctatctcatctttcttgctaccgaaatctttcgcattcttgttaatcttctgaccctcccttccagatcgagagagagaccgccgccgctgtgtctgcctctggtgctgtgttctgctctgccgtgaagcgccaaaggtgggcttggtccagatctagccaagtttaagtttgtaactcagttctggggcttggactgcattacagctcatcatattcatcgatctgcaggtttctagctgattggttgcaaagagagctgctcatggaggtcgtcacggggggcatgggcagcctgctccccaagctgggcctgctgctcatggatgagtacaacttgcacaagcgcgtcaagaaagatgtcgagttcctcagcaaggagcttgagagcatgcacgctgccctcatcaaggttggcgacgtgccgcgggaccagctcgacaggcaggtcaagctctgggccggcgaggtcagagagctctcctacgacatggaggatgtcgtcgacaagttcctcgtacgtgttgacggcattcatcctcacgacgatgcaaacagattcaaggggctcatgaagaggatgatcggcgtgttcaagaaaggcaagaatcaccatcgaatagctgacgcgatcaaggacatcaaggaggaactcaaggaggtggctgctaggcgggacaggaacaaggtcgatggtagtgctcctaatcctataaaagcaatacctatcgatcctcgtcttcgtgctctctacatagaggcgacagagcttgttggcatctatgggaagagggaccaagacctcttgaggttgctctccattgagggcgacgacgcatgtaacaagagactgaagaagatctccattgttggatttggtgggttgggaaagaccactcttgctagagcagtatatgagaagattaaaggtgtttttgattgtcgggcatttgtccccgttggtcagagccctgacatcaagaaggtttttaaggatatcctcattgatctcggaaaatcccactcagatgttgccatgttggataataggcagcttatcaacaagcttcatgaattcctcgagaacaagaggtatgcatcacttgcagcaaaaatgacactattgtgatatgtttgtttcctatgccaattgtagcctcgcatgaaagggtctggggtaaaagaaatcattagggtcacctctgctgatatatgtagtatgcactaaaccgtgcaaaaaaatatcagtagggtcaccttcgccgatatatgtacttgtatagatcagattagtttttcttatctagtaaggtaattcggttaacttacaactgcagctttttttagcatcctgaaaacagagtccaaatcagagaacttgtgctcttgctactaatagtaaacatcataggtgctaatttcaagactttataaatatacatctttgaccctcatctgttttatctaagtagatggtgagaacttctagttgcaatcgttagggtggctgaaatttattgccattggtttaagaaatccatctcgacataccaaagataccaaataatgagcataatgaaaattgtagcaaataatacaccagaaagtaaaggaaagggaaggagagataaactttacaatatttaacttcaagtttctttctttctcctaacccacctcattctaaaataaatatgttaaagtttgcatctcgcaacaaaggctataccctagaattttcctaagatactgtatgctggattgatgaccgaacaggaagtttaatttgctcttatcacattctattttctctttatgctcctttcggaaacttactaccaacaatatgcatgaaacgataatgacatgcattctgagtctcaaggatgttttttagcggatgtatataaattggtggtccaatcagtactcgggtctttccaatagatctacctaaaccattgaaatatttgaaatgttgtaactaattaaaagttcttctatatacggccatctatccaagagtttgctctttgtgtcttactcctagaatcttaaatttgtattcattttgttcaacatctgattgtcagttcataagattgtcgagattgagactcgtatttcttattagtttgtttctgaacatacacattctgcatacatggttccgctcacatattcataacgcccatatatagcttttccagttcaatagatcttacactaatactccgaactggtgtaggtacctcatcataattgatgatatatgggatcaaaacttgtggaaagacatcaacattgctttctctatcatgaacaatttaggcagtcggctaatcaccacaacccgcattctcaatgtatctgaatcatgttgctcgtcgtccaacgattcaatctatcaaatggaaccgctttctacagatgactcccgaaggcttttctgtaaaagaatatttcctagcgatactggatgtccaaatgaatttgaacaagtgtctagggatattctgaagaaatgtggtggggtgccactagccataattactatagctagtgctttggctggtggcgagaaggtgaaacctaaacatgagtgggatattctgctgcagtcccttggttctggactaacagaagataacagcttagatgagatgcggagaatactatcttttagctattatgatctaccttctcatctgagaacttgtctattgtatctaagtatataccccgaagatagagagattgatagagatagtctgatatggaagtgggtagccgaaggatttgtccatcatggaaatcaagggaccagcttatttttgctcgggttagattacttcaaccagctcattaatagaagtatgatccagccgatatatgatactattggcgaggtatatgcttgccgtgtacatgatatggttttggaccttatctgcaacttgtcatataaagcaaagtttgtgaatctattggatggcactgggaatagcatgtcttcacagagtaattgtcgccgtttgtcccttcagaaaagaaatgaagatcatcaagccaggcctctcacagatatcaagagtatgtcacggatgaggtcaattactatctttccacctgctattaaactcatgccatctctttcaaggtttgaggttttacgtgtacttgatttgttgggatgtaatcttgggaagaataccaacctgcagcttaatctcaaggatgttgggcatttaattcacctaaggtaccttggtctcgaagataccaaaatcagtaagctcccggctgagataggaaaactgcagtttttgaaggtgttggatcttggaagaaattataatctaaatgaattgtcgtccactgtttgtaatttcagaagattaatctacctaaatttagttggctgtcaggtggttcctccagttggtgtgttgcaaaatctgagagccacagaagtgttgaggggtatcttggtctctctgaacattattgcacaagagcttggcaacttgaaaaggctgagggagcttgagatttgtttcacggatggtagtttggatttgtatgagggtttcgtgaagtctctgtgcaacctacatcacattgaaagtctatgtattcatgactattccgaagaaacatcatcttttgaactgatggttctcttgggagaacgctgggtgcctcctgtacatttccgtgaatttctgtcatccatgcctagccaactctctgcactgcgagggtggataaagagagacccctcccatctctcgaacctcttcgagttaatcctctggcaagtgaaggaagtgcagcaggaggacgtggaaatcattgggaggttgcggtcccttcgttgtctctggataaaagagagcacccaccaaacgcaacggctgctagtcatccgtgcagatgggttccgttgtatggttaactttgaattggattgtggatctgccacgcagatattgtttgagcctggagctttgctgagggcggaagcggtttcgttcagtcttggcgtgcgggtggcgaaagaggatggcaactgtggtttcgatttgggcctgcaggggaacctgctatcccttcggcaacctgctatccattgtggtggagtgagggttggggaggcaaaggaagcggaggctgctgtgaggcacgccctcgacgcccatcccaaccatcccgcgattgcaatttccatgttcccgtatatagcagaaggtacgtactcatgctgcacccaactaattacttatgcatttacgagtgtgttctctcatgagcgtgtgaccttattactttctgcattgatttgatctctgaatctcccaaggtgctcaagatgacgatttaatgtgattacctggtttgaggatcgacttctgatgcagattgactgacattgcttcaggtgtgatctcaatctgtacatatttactcatcatattattctcgaccctcctgttttccatcgctagacctcagcttgtaattgtattgttcaattgtgcttctctaatacctactgaaattatgaacaggcaaaggtaatgtcatagaagtttctttggcataagaatctgagaaaaatgcactttggcgcaacttacctttttgtagtagaattgctcgtcatactatgtggaattgtgtgattgggtcatgtatttcagtgtggttctaatgaggaagtggcagttatttggtctatttgaaaaatgagaaatgatgattgcttaagaaatatttgccatacatacctcagtcgtgtagttgctaaagttgcgttttggatgttctgttggtctagcttacacagcaaagccttgcactgtgcaggtaccagcggcgtctgagatgtttcgcaaccgttgggtgggcaccagtcatgcaaaaatttgaaggatgaaaaggttccgagttttgctggaagttgtgtgatccgctcctactgatgggatggctggtttgtcgtttgcataaagaaatgttgcttgttttttcttctaacggactaagtaaaagtgttgtagtaagttgctctgttgtttatgagagccatttaaaagtgttgtagtgttgatgcttttttttatctcaaccggacaaacctaagggtgta

SEQ ID NO: 51 - DNA, 2853 bp, Hordeum vulgare
atggagctggtggctggtgccatggttagcctgatctccaagcttggcaaattgcttacggaggagtacaacctgcgcaagagtgtcaaaaaaaatgtcgagttcctccggagggagcttgagatcatgcacactgtcctcatcaaggttgacgaggtgccgcgggagcagctcgacagtcaagttaagatctgggccgacgaggtcagagagctctcctacaacatggaggatgtcatcgacaagtgcctcgtacgggttgacaacattcagtctcacgacaatgcaaacggtttggagaggctcatgaagaggatgattgtcgtgttcaagaaaggcaagaatcaccatcgaatagctaatgcgattacggaaatcacggagcaattccatgagttggctgctaggcctgaaaggaacaaggttgatggtattactcctaatcctacataagcaattgttctcgatcctcgtctacgtgctctgtacacagaagtgacggagctcgttggcatctctgggaagagggatgaagacatcatgagattgctttccatggagaccgaagacgatgcctctaacaagagactcaaaaaggtctctattgttggatttggagggttgggcaagaccactcttgctaaggcagtatacgagaagattaaaggtgatttcgattgtcgggcatttgtccccatcggtcgcaaccctgacatcaagaaggttttcagggatatcctcattgaactcggcaactctcactcagatcttacgatacttgatgcgaagcagcttatggtcaagcttcgtgaattcctcgagaacaagaggtatctcgtcataattgatgatatatgggatgaaagcttgtgggaaatcatcaagtttgctttctccaacaggaataatctaggcagtcggctaatcaccacaacccgcattgtcagtgtctccaattcatgttgctcgtcagctgatgattcagtttatcaaatgaaacctctttctcttgatgactccagaaagctcttccataaaagaatattttccagcgagactgaatgtccaaatgaatttgaacaagtgtctagagatattctaaagaaatgtggtggggtgccactagccataattactatagctagtgctttggctggtggccagaaggtgaaaccaaagcatgagtggtatattctgctgcagtcccttggctctggactaacagaagataacagtttagatgagatgcggaggatactatctttcagctattatgatctaccttatgatctgagaacttgtctattgtatctatctatatacccagaaggtagtgagattggtagagatagactgatatggaagtgggtggccgaaggatttgtccaccctggaaatcaagggacaagcctgtttttgctcggattaaattacttcaaccagctcattaatagaagtatgatccagccaatatatgatcatttaggccagatatctacttgccgtatacatgatatggttctggaccttatctgcaacttgtcacatgaagcaaagtttgttaatctattggacggcaccaggaatagcatgtcttcacaaagtaatgttcgtcgtttgtcccttcagaatataaatgaagatcatccagctaaatctctcacaaatatcatgagtatgtcacgagtgaggtcaattactatctttccaactgctattgatatcatgccagctctttcaaggtttaaggctttacgtgtacttgatctgatgggatgtaatcttggggaaaatagcaacctgcaacttcacctcaaggatgtaggacatttaattcacctaaggtacctaggtctatcacgtaccaaaattagggaactcccgcccaagataggaaatctgcagtttttggaggtcttggatcttggaaacaattatatagatgaattgccgcccactgtttgtaatttgagaagattaatctacctaaacatttatccctgtaaggtggttccaactggtgtgttgcacaatttgacatccatggaagtgctgagggagatcttggtccctctgaacattattgcacaagagcttggcaacctggcaaggctgagggagcttaggattcacttcaaggatggtcattatgatttgtatgaaggatttgtgaagtctctgtgcaacctacatcgcatggaaagtctaagtattgattgcaattatggagaaacatcttttgaactcatggatctcttggcagaacgctgggtgcctcctgtacatctccgcgaatttgtgtcacggatgcccagcaaactctctgcactgcgagggtggataaagaaagacccctcgcatctctcaaacctctcagtgttattcctctggccagtgaaggaagtgcagcaggaggacgtggaaatcattggggggttgcagtcccttcgccgtctatggatgaagagcacccaacaaatacaacggctgctaatcatccgtgcagatgtgttcctctgtatggtagactttgggttgtattgtggatcagcagcgcagataatgtttgaaccaggagctttaccgagggctgaatatgttaggttcagccttggcgtgcgggtggccaaagaggatggtaactatggtttcgacttgggcctgcaggggaacctgctctcccttcggcagcgtgtctgggttaatctgtattgtggtggagcgagggttggggaggcaaaggaagcggaggctgctgtgaggcacgccctcgacgcccatcccaaccattccgcggttgaaatttacatgttcccgcatatagcagaaggtactcacgctgcacccaactaa

SEQ ID NO: 52 - DNA, 2853 bp, Hordeum vulgare
atggagctggtggctggtgccatggttagcctgatctccaagcttggcaaattgcttacggaggagtacaacctgcgcaagagtgtcaaaaaaaatgtcgagttcctccggagggagcttgagatcatgcacactgtcctcatcaaggttgacgaggtgccgcgggagcagctcgacagtcaagttaagatctgggccgacgaggtcagagagctctcctacaacatggaggatgtcatcgacaagtgcctcgtacgggttgacaacattcagtctcacgacaatgcaaacggtttggagaggctcatgaagaggatgattgtcgtgttcaagaaaggcaagaatcaccatcgaatagctaatgcgattacggaaatcacggagcaattccatgagttggctgctaggcctgaaaggaacaaggttgatggtattactcctaatcctacataagcaattgttctcgatcctcgtctacgtgctctgtacacagaagtgacggagctcgttggcatctctgggaagagggatgaagacatcatgagattgctttccatggagaccgaagacgatgcctctaacaagagactcaaaaaggtctctattgttggatttggagggttgggcaagaccactcttgctaaggcagtatacgagaagattaaaggtgatttcgattgtcgggcatttgtccccatcggtcgcaaccctgacatcaagaaggttttcagggatatcctcattgaactcggcaactctcactcagatcttacgatacttgatgcgaagcagcttatggtcaagcttcgtgaattcctcgagaacaagaggtatctcgtcataattgatgatatatgggatgaaagcttgtgggaaatcatcaagtttgctttctccaacaggaacaatctaggcagtcggctaatcaccacaacccgcattgtcagtgtctccaattcatgttgctcgtcagctgatgattcagtttatcaaatgaaacctctttctcttgatgactccagaaagctcttccataaaagaatattttccagcgagactgaatgtccaaatgaatttgaacaagtgtctagagatattctaaagaaatgtggtggggtgccactagccataattactatagctagtgctttggctggtggccagaaggtgaaaccaaagcatgagtggtatattctgctgcagtcccttggctctggactaacagaagataacagtttagatgagatgcggaggatactatctttcagctattatgatctaccttatgatctgagaacttgtctattgtatctatctatatacccagaaggtagtgagattggtagagatagactgatatggaagtgggtggccgaaggatttgtccaccctggaaatcaagggacaagcctgtttttgctcggattaaattacttcaaccagctcattaatagaagtatgatccagccaatatatgatcatttaggccagatatctacttgccgtatacatgatatggttctggaccttatctgcaacttgtcacatgaagcaaagtttgttaatctattggacggcaccaggaatagcatgtcttcacaaagtaatgttcgtcgtttgtcccttcagaatataaatgaagatcatccagctaaatctctcacaaatatcatgagtatgtcacgagtgaggtcaattactatctttccaactgctattgatatcatgccagctctttcaaggtttaaggctttacgtgtacttgatctgatgggatgtaatcttggggaaaatagcaacctgcaacttcacctcaaggatgtaggacatttaattcacctaaggtacctaggtctatcacgtaccaaaattagggaactcccgcccaagataggaaatctgcagtttttggaggtcttggatcttggaaacaattatatagatgaattgccgcccactgtttgtaatttgagaagattaatctacctaaacatttatccctgtaaggtggttccaactggtgtgttgcacaatttgacatccatggaagtgctgagggagatcttggtccctctgaacattattgcacaagagcttggcaacctggcaaggctgagggagcttaggattcacttcaaggatggtcattatgatttgtatgaaggatttgtgaagtctctgtgcaacctacatcgcatggaaagtctaagtattgattgcaattatggagaaacatcttttgaactcatggatctcttggcagaacgctgggtgcctcctgtacatctccgcgaatttgtgtcacggatgcccagcaaactctctgcactgcgagggtggataaagaaagacccctcgcatctctcaaacctctcagtgttattcctctggccagtgaaggaagtgcagcaggaggacgtggaaatcattggggggttgcagtcccttcgccgtctatggatgaagagcacccaacaaatacaacggctgctaatcatccgtgcagatgtgttcctctgtatggtagactttgggttgtattgtggatcagcagcgcagataatgtttgaaccaggagctttaccgagggctgaatatgttaggttcagccttggcgtgcgggtggccaaagaggatggtaactatggtttcgacttgggcctgcaggggaacctgctctcccttcggcagcgtgtctgggttaatctgtattgtggtggagcgagggttggggaggcaaaggaagcggaggctgctgtgaggcacgccctcgacgcccatcccaaccattccgcggttgaaatttacatgttcccgcatatagcagaaggtactcacgctgcacccaactaa

SEQ ID NO: 53 - DNA, 2877 bp, Hordeum vulgare
atggatattgtcaccggtgccatttccaacctgattcccaagttgggggagctgctcacggaggagttcaagctgcacaagggtgtcaagaaaaatattgaggacctcgggaaggagcttgagagcatgaacgctgccctcatcaagattggtgaggtgccgagggagcagctcgacagccaagacaagctctgggccgatgaagtcagagagctctcctacgtcattgaggatgtcgtcgacaagttcctcgtacaggttgatggcattcagtttgatgataacaacaacaaatttaaggggttcatgaagaggacgaccgagttgttgaagaaagtcaagcataagcatgggatagctcacgcgatcaaggacatccaagagcaactccaaaaggtggctgataggcgtgacaggaacaaggtatttgttcctcatcctacgagaacaattgctattgacccttgccttcgagctttgtatgctgaagcgacagagctagttggcatatatggaaagagggatcaagacctcatgaggttgctttccatggagggcgatgatgcctctaataagagactgaagaaggtctccattgttggatttggagggttgggcaagaccactcttgctagagcggtatacgagaagattaaaggtgatttcgattgtcgggcttttgttccggtcggtcagaaccctcacatgaagaaggttttaagggatatcctcattgatctcggaaatcctcactcagatcttgcgatgctggatgccaatcagcttattaaaaaacttcgtgaatttctagagaacaaaaggtatcttgtcataattgatgatatatgggatgaaaaattatgggaaggcatcaactttgctttctccaataggaataatctaggcagtcggctaatcaccacaacccgcattgtcagtgtctctaattcatgttgctcatcacatggtgattcggtttatcaaatggaaccactttctgttgatgactccagaatactcttctggaaaagaatatttccagatgagaatggatgtctaaatgaatttgaacaagtgtcgagagatattctaaagaaatgtggtggggtaccactagccataattaccatagctagtgctttggccggtgaccagaagatgaaaccaaagtgtgagtgggatattctccttcagtcccttggctctggactaacagaagataacagtttagaggagatgcggagaatactctctttcagctattctaatctaccttctcatctgaaaacttgtctactgtatctatgtatatatccagaagatagcaagattcatagagatgaactgatatggaagtgggtggccgaaggatttgtccaccatgaaaaccaaggaaatagcttgtatttgctcggattaaattacttcaaccagctcattaatagaagtatgatccagcccatatatggttttaatgacgaggtatatgtatgtcgtgtacatgatatggttctggaccttatctgcaacttgtcacgtgaagcaaaatttgtgaatctattggatggcagtgggaatagcatgtcttcacagggtaattgtcgccgtctgtcccttcaaaaaagaaatgaagatcatcaagccaaacctatcacagatatcaagagtatgtcacgagtgaggtcaattactatctttccacctgctattgaagtcatgccatctctttcaaggtttgacgttttacgtgtacttgatctgtcacgatgtaatcttggggagaatagcagcctgcagcttaacctgaaggatgttggacatttaactcacctaaggtaccttggtctagaaggtaccaacatcagtaagctccctgctgagataggaaaactgcagtttttggaggtgttggatcttggaaacaatcataatctaaaggaattgccgtccactgtttgtaatttcagaagattaatctacctaaatttatttgggtgtccggtggttcctccagttggtgtgttgcaaaatctgacatccatagaagtgttgagggggatcttggtctctgtgaacattattgcacaagagcttggcaacctggaaaggctgagggtgcttgatatttgcttcagggatggtagtttggatttgtataaagatttcgtgaagtctctgtgcaacctacatcacatcgaaagtctacgtattgagtgcaattccagagaaacatcatcttttgaactggtggatctcttgggagaacgctgggtgcctcctgtacatttccgtgaatttgtgtcatccatgcctagccaactctctgcactgcgagggtggataaagagagacccctcccatctctcgaacctctccgagttaatcctctcgtcagtgaaggacgtgcagcaggatgacgtggaaatcattggggggttgttgtgccttcgtcgtctctttataataacgagcaccgaccaaacgcaacggctgctagtcatccgtgcagatgggttccgctgtacggttgactttcgattggattgtggatctgccacgcagatattgtttgaaccaggagctttgccaagggcggtaagagtttggttcagccttggcgtgcgggtgacgaaagaggatggtaaccgtggcttcgacttgggcctgcaggggaacctgttctcccttcgagagtttgtctctgtttatatgtattgtggtggagcgagggttggggaggcaaaggaagcggaggctgcggtgaggcgtgccctggaagctcatcccagccatccccggatttatattcagatgaggccgcatatagcaaaaggtgctcatgatgacgatttgtgtgaggacgaggaggagaactga

SEQ ID NO: 54 - DNA, 2871 bp, Hordeum vulgare; identical to SEQ ID NO: 41

SEQ ID NO: 55 - DNA, 2851 bp, Hordeum vulgare
atggaggtcgtcacggggggcatgggcagcctgctccccaagctgggcctgctgctcatggatgagtacaacttgcacaagcgcgtcaagaaagatgtcgagttcctcagcaaggagcttgagagcatgcacgctgccctcatcaaggttggcgacgtgccgcgggaccagctcgacaggcaggtcaagctctgggccggcgaggtcagagagctctcctacgacatggaggatgtcgtcgacaagttcctcgtacgtgttgacggcattcatcctcacgacgatgcaaacagattcaaggggctcatgaagaggatgatcggcgtgttcaagaaaggcaagaatcaccatcgaatagctgacgcgatcaaggacatcaaggaggaactcaaggaggtggctgctaggcgggacaggaacaaggtcgatggtagtgctcctaatcctataaaagcaatacctatcgatcctcgtcttcgtgctctctacatagaggcgacagagcttgttggcatctatgggaagagggaccaagacctcttgaggttgctctccattgagggcgacgacgcatgtaacaagagactgaagaagatctccattgttggatttggtgggttgggaaagaccactcttgctagagcagtatatgagaagattaaaggtgtttttgattgtcgggcatttgtccccgttggtcagagccctgacatcaagaaggtttttaaggatatcctcattgatctcggaaaatcccactcagatgttgccatgttggataataggcagcttatcaacaagcttcatgaattcctcgagaacaagaggtacctcatcataattgatgatatatgggatcaaaacttgtggaaagacatcaacattgctttctctatcatgaacaatttaggcagtcggctaatcaccacaacccgcattctcaatgtatctgaatcatgttgctcgtcgtccaacgattcaatctatcaaatggaaccgctttctacagatgactcccgaaggcttttctgtaaaagaatatttcctagcgatactggatgtccaaatgaatttgaacaagtgtctagggatattctgaagaaatgtggtggggtgccactagccataattactatagctagtgctttggctggtggcgagaaggtgaaacctaaacatgagtgggatattctgctgcagtcccttggttctggactaacagaagataacagcttagatgagatgcggagaatactatcttttagctattatgatctaccttctcatctgagaacttgtctattgtatctaagtatataccccgaagatagagagattgatagagatagtctgatatggaagtgggtagccgaaggatttgtccatcatggaaatcaagggaccagcttatttttgctcgggttagattacttcaaccagctcattaatagaagtatgatccagccgatatatgatactattggcgaggtatatgcttgccgtgtacatgatatggttttggaccttatctgcaacttgtcatataaagcaaagtttgtgaatctattggatggcactgggaatagcatgtcttcacagagtaattgtcgccgtttgtcccttcagaaaagaaatgaagatcatcaagccaggcctctcacagatatcaagagtatgtcacggatgaggtcaattactatctttccacctgctattaaactcatgccatctctttcaaggtttgaggttttacgtgtacttgatttgttgggatgtaatcttgggaagaataccaacctgcagcttaatctcaaggatgttgggcatttaattcacctaaggtaccttggtctcgaagataccaaaatcagtaagctcccggctgagataggaaaactgcagtttttgaaggtgttggatcttggaagaaattataatctaaatgaattgtcgtccactgtttgtaatttcagaagattaatctacctaaatttagttggctgtcaggtggttcctccagttggtgtgttgcaaaatctgagagccacagaagtgttgaggggtatcttggtctctctgaacattattgcacaagagcttggcaacttgaaaaggctgagggagcttgagatttgtttcacggatggtagtttggatttgtatgagggtttcgtgaagtctctgtgcaacctacatcacattgaaagtctatgtattcatgactattccgaagaaacatcatcttttgaactgatggttctcttgggagaacgctgggtgcctcctgtacatttccgtgaatttctgtcatccatgcctagccaactctctgcactgcgagggtggataaagagagacccctcccatctctcgaacctcttcgagttaatcctctggcaagtgaaggaagtgcagcaggaggacgtggaaatcattgggaggttgcggtcccttcgttgtctctggataaaagagagcacccaccaaacgcaacggctgctagtcatccgtgcagatgggttccgttgtatggttaactttgaattggattgtggatctgccacgcagatattgtttgagcctggagctttgctgagggcggaagcggtttcgttcagtcttggcgtgcgggtggcgaaagaggatggcaactgtggtttcgatttgggcctgcaggggaacctgctatcccttcggcaacctgctatccattgtggtggagtgagggttggggaggcaaaggaagcggaggctgctgtgaggcacgccctcgacgcccatcccaaccatcccgcgattgcaatttccatgttcccgtatatagcagaaggtgctcaagatgacgatttaa

SEQ ID NO: 56 - DNA, 2833 bp, Hordeum vulgare
atggaggtcgtcacgggtgccatgggcagcctgctccccaagcttggccagctgctcatggatgagtacaacctgcacaagcgcgtcaagaaagacgtccgcttcctctccagggagcttgagagcatgcacgctgccctcgtcaaggttggcgacgtgccgcgggatcagctcgacacacaagttaagctctgggccgacgaggtcagagaactctcctacgacatggaggatgtcgtcgacaagttcctcgtacgtgttgacggcattcatcctcacgacgatgcaaacagattcaaggggctcatgaagaggatggtcggcgtgttcaagaaaggcaagaatcaccatcgaatagctgacgcgatcaaggacatcaaggagcaactccaggaggtggctgctaggcgggacaggaacaaggtcgatggtagtgctcctaatcctataaaagcaatacctatcgatcctcgtcttcgtgctctctacatagaggcgacagagcttgttggcatctatgggaagagggatcaagacctcttgaggttgctctccattgagggcgacgacgcatgtaacaagagactgaagaagatctccattgttggatttggtgggttgggaaagaccactcttgttagagcagtatacgagaagattaaaggtgtttttgattgtcgggtatttgtccccgttggtcagaaccctgacatcaagaagatttttaaggatatcctcattgatctcggaaaatcccactcagatgttgccatgttggatgaaaggcagcttatcaacaagctgcatgaattcctcgagaacaagaggtatctcatcataattgatgatatatgggatcaaaacttgtggaaagacatcaacattgctttctccaacaggaacaatttaggcagtcggctaattaccacaacccgcattctcaatctatctgagtcatgttgctcgtcgtccgacgattcaatttatcaaatggaaccgctttctacagatgactcccgaaggcttttctgtaaaagaatatttcctagcgagactgtatgtccaaatgaatttgaacaagtgtctagggatattctaaagaaatgtggtgaggtgccactagccataattactatagctagtgctttggctggtggcgagaaggtgaaaccaaagcatgagtgggatatactgttgcagtcccttggctctggactaacagaagataacagcttagatgagttgcggagaatactatcttttagctattataatctaccttttcatctgagaacttgtctattgtatctaagtatataccccgaagatagcatgattgatagagatagtctgatatggaagtgggtggccgaaggatttgtccaccatggaaatcaagggactagcctgtttttgctcggattaaatttcttcaaccagctcattaatagaagtctgatccagccaatatatagttttagtggtgatgtacatgcttgtcgtgtacatgatatggttctggaccttatctgcaacttgtcacatgaagcaaaatttgtgaatctattggatggcactgggaatagcatgttttcacagagtaattgtcgccgtttgtcccttcaaaatagaaatgaagatcatcaagccaagcctctcacagatatcaagagtatgtcacgagtgaggtcaattactatctttccacctgctattgaagtcatgccatctctttcaaggtttgacgttttacgtgtacttgatctatcgaaatgtattcttggggagaatagcagccttcagcttaacctcgaggatgttggacatttaattcacctaaggtaccttggtctagaaggtaccaaaatcagtaagctcccggctgagataggaaaacttcagtttttggaggtgttggatcttgaagacaatcataatctaaatgaattgccgttcactgtttgtaattttagaagattaacctacctaaatttagttggctgtcaggtgtttcctctagttggtgtgttgcaaaatctgacatccatagaagtgttgagggggatctgggtctctctgaaaattattgcacaagagcttggcaacctggaaaggctgagggagcttgagatttactgaattcgtgaattctctgtgcaacctacatcacatcgaaagtctacgtattgagtgcaattccagagaaacatcatcttttgaacttatggatctcttgggggaacgctggctgcctcctgtacatctccgcagatttgtgtcttccatgcccagccaactctctgcactgcgagggtggataaagagagacccctcacatctctcgaacctctccgagttaatcctctcgtcagtgaaggacgtgcagcaggatgacgtggaaatcattggggggttgttgtgccttcgtcgtctctttataataacgagcaccgaccaaacgcaacggctgctagtcatccgtgcagatgggttccgctgtacggttgactttcgattggattgtggatctgccacgcagatattgtttgaaccaggagctttgccaagggcggtaagagtttggttcagccttggcgtgcgggtgacgaaagaggatggtaaccgtggcttcgacttgggcctgcaggggaacctgttctcccttcgagagtttgtctctgtttatatgtattgtggtggagcgagggttggggaggcaaaggaagcggaggctgcggtgaggcgtgccctggaagctcatcccagccatccccggatttatattcagatgaggccgcatatagcaaaaggtacgcatcctgcacctaactaa

SEQ ID NO: 57 - DNA, 2904 bp, Hordeum vulgare
atgcatgtggtgactggtgccatgggaagcctgctccccaagctgggccagttgctcatggaggagtacaagctgcacaaacgcgtcaagaatgatgtcgagttcctgcggaaggagcttgagagcatgcacactgcccttatcaaggttggtgaggtaccgcggcaccagctcgacaagcaagtcaagctctgggccgatgaagttagagacctctcctacaacatggaggatgtggttgacaagttcctcgtacgtgtcgatggcgttgatcctcacgacaacaccgacagattcaaggggatcatgaggaagatgattggcttgttcaagaaaggcaagaatcaccatcagatagctgacgctatcaaggagatcaaggagcaactccaggaggtggccgctaggcgtgacaggaacaaggtcgagggtattgcctctaatcccatggaagcaatacctatcgatccttgtcttagagctttgtatgctgaagcgacagagctagttggtatctacgggaagagggatgaggacatcatgcggttgctatccatggagggcgaggacgatgcctctaacaagagactgaaaaaggtctccatcgttggatttggtgggttaggcaagaccactcttgccaaagcagtatacgaaaatatcaaaggtgattttgattgtcgggcatttgtccccgtcggtcagaaccctgacatgaagaaggtttttagggatatcctcatagatctccgtgtgtctaactcagagcttgcggaattggatgaaaggcagcttatcaacaagcttcatgaatttctcgagaacaagaggtatcttgtcatcattgatgacatatgggatgcaaaattgtgggaaagaatcaactttgctttctctaacaggaacaatttaggcagtcgtctaataatcacaacccgcattttcagtgtctccaaatcaagttgcatgttgcctgatgatgcagtttatgaaatgaaacctctttctgatgatgactccagaagtctcttctataaaagaatatttcctagcgagagtggatgtccaaatgaatttgaacaagtgtctgaagacattttgaagaagtgtggtggggtaccactagctatcattactatagctagttctttggctagtggccaaaaggtaaagccaaagagtgaatgggacatcctcctccagtcccttggctctggactaacaaaagataacagtttagaggagatgcggagaatactctctttcagctattatgatctacccgatcatctgaaaacttgtttgttgtacctatgtatatatccagaagatagcatgattgatagagatagacttatatggaagtgggtggccgaaggatttatccaccagggaaatcaagggactagcttgtttttgctcggattaaattacttcaaccaacttattaatagaagtatgatccagccaatatatgatggtctaggcgaggtatctgcttgtcgtgttcatgatatggttctggaccttatctgcaacttgtcacatgaagcaaaatttgtcaatgtattgaatggcacacgagatagcatgtcttctcagagcaatgttcgtcgtttgtcccttcaagatggaagtaaagatcaccaaggcagacctctcaggaatttcacgggtatatcacgagtgaggtcaattactatcttcccacctgctattaatatcatgccagccctgtcaaggcttgaagttttacgtgtacttgatctatatcactgtaatcttgggaaaaatagcagcctgcagcataggcttagggatgttggacatctgattcacctaaggtacctaggcctagcaggtactaaaattagtgaactcccggctgagattggaaacctacagtttctagaggtgttggatcttgaagacaattctgaactacgtaacttgtcgtcgactatttgtaagttgagaagattaatctgcctacatgttcatagggatgaggtggctccgggtgtgttgcagaatctcacatccattgaagtgttgaggaggctcgttgtgtctctgaatactgttgcacaagagcttggcaacctagtaaggctgagggagcttctagtttgcttcatcaatgttggtttggatttgtatgaaggttttgtgaagtctgtgtgcaacctacatcacatcgaaagtcttcgtatctacagtgtaagagcatcttcagaactcatggatctcttgggagaacgatgggttcctcctggacatctccgcagatttgaggcacacatgcctagccaactttcggcactacgagggtggataatgagagaccccttgcatctctcgaacctctccgatttagtcctcacgtcagtaaaggaagtgcaacaagaggacatggaaataattgggggcttgttgtcccttcgaggtctccagataaagagcacccagcaaacgcaacggctgctagtcatccgtgcagatgtgttccgttgtatgatatgctttgacttggattgtggatcaggagcgcaaatagtgtttgaaccaggagctttgccgaggacggaaggacttaggttcagcctcggcgtgcgggtgacgaaagaggatggtaaccatggcttcgacttgggcctgcaggggaacctgctctcccttcggacgtttgtgtgggttcaaatctattgtggtggagcgagggtgggggaggcaaaggaagcggatgctgtggtgaggcacacactcaggtcccatcccaaccatcccgggattatatcccttatgtttaatatgatcccgaatatagcagaaggtactcaccagtcaccccgcaccctactaattaattactcgtgcacttatgtatgtgctttttctcaatga

SEQ ID NO: 58 - protein, 956 aa, Hordeum vulgare
MDIVTGAISNLIPKLGELLTEEFKLHKGVKKNIEDLGKELESMNAALIKIGEVPREQLDSQDKLWADEVRELSYVIEDVVDKFLVQVDGIQSDDNNNKFKGLMKRTTELLKKVKHKHGIAHAIKDIQEQLQKVADRRDRNKVFVPHPTRPIAIDPCLRALYAEATELVGIYGKRDQDLMRLLSMEGDDASNKRLKKVSIVGFGGLGKTTLARAVYEKIKGDFDCRAFVPVGQNPDMKKVLRDILIDLGNPHSDLAMLDANQLIKKLHEFLENKRYLVIIDDIWDEKLWEGINFAFSNRNNLGSRLITTTRIVSVSNSCCSSDGDSVYQMEPLSVDDSRMLFSKRIFPDENGCINEFEQVSRDILKKCGGVPLAIITIASALAGDQKMKPKCEWDILLRSLGSGLTEDNSLEEMRRILSFSYSNLPSHLKTCLLYLCVYPEDSMISRDKLIWKWVAEGFVHHENQGNSLYLLGLNYFNQLINRSMIQPIYNYSGEAYACRVHDMVLDLICNLSYEAKFVNLLDGTGNSMSSQSNCRRLSLQKRNEDHQVRPFTDIKSMSRVRSITIFPSAIEVMPSLSRFDVLRVLDLSRCNLGENSSLQLNLKDVGHLTHLRYLGLEGTNISKLPAEIGKLQFLEVLDLGNNRNIKELPSTVCNFRRLIYLNLVGCQVVPPVGLLQNLTAIEVLRGILVSLNIIAQELGKLKSMRELEIRFNDGSLDLYEGFVKSLCNLHHIESLIIGCNSRETSSFEVMDLLGERWVPPVHLREFESSMPSQLSALRGWIKRDPSHLSNLSDLVLPVKEVQQDDVEIIGGLLALRRLWIKSNHQTQRLLVIPVDGFHCIVDFQLDCGSATQILFEPGALPRAESVVISLGVRVAKEDGNRGFDLGLQGNLLSLRRHVFVLIYCGGARVGEAKEAKAALRRAQEAHPDHLRIYIDMRPCIAEGAHDDDLCEGEEEN

SEQ ID NO: 59 - protein, 958 aa, Hordeum vulgare
MDIVTGAISNLIPKLGELLTEEFKLHKGVKKNIEDLGKELESMNAALIKIGEVPREQLDSQDKLWADEVRELSYVIEDVVDKFLVQVDGIQFDDNNNKFKGFMKRTTELLKKVKHKHGIAHAIKDIQEQLQKVADRRDRNKVFVPHPTRTIAIDPCLRALYAEATELVGIYGKRDQDLMRLLSMEGDDASNKRLKKVSIVGFGGLGKTTLARAVYEKIKGDFDCRAFVPVGQNPHMKKVLRDILIDLGNPHSDLAMLDANQLIKKLREFLENKRYLVIIDDIWDEKLWEGINFAFSNRNNLGSRLITTTRIVSVSNSCCSSHGDSVYQMEPLSVDDSRILFWKRIFPDENGCLNEFEQVSRDILKKCGGVPLAIITIASALAGDQKMKPKCEWDILLQSLGSGLTEDNSLEEMRRILSFSYSNLPSHLKTCLLYLCIYPEDSKIHRDELIWKWVAEGFVHHENQGNSLYLLGLNYFNQLINRSMIQPIYGFNDEVYVCRVHDMVLDLICNLSREAKFVNLLDGSGNSMSSQGNCRRLSLQKRNEDHQAKPITDIKSMSRVRSITIFPPAIEVMPSLSRFDVLRVLDLSRCNLGENSSLQLNLKDVGHLTHLRYLGLEGTNISKLPAEIGKLQFLEVLDLGNNHNLKELPSTVCNFRRLIYLNLFGCPVVPPVGVLQNLTSIEVLRGILVSVNIIAQELGNLERLRVLDICFRDGSLDLYKDFVKSLCNLHHIESLRIECNSRETSSFELVDLLGERWVPPVHFREFVSSMPSQLSALRGWIKRDPSHLSNLSELILSSVKDVQQDDVEIIGGLLCLRRLFIITSTDQTQRLLVIRADGFRCTVDFRLDCGSATQILFEPGALPRAVRVWFSLGVRVTKEDGNRGFDLGLQGNLFSLREFVSVYMYCGGARVGEAKEAEAAVRRALEAHPSHPRIYIQMRPHIAKGAHDDDLCEDEEEN

SEQ ID NO: 60 - protein, 949 aa, Hordeum vulgare
MEVVTGGMGSLLPKLGLLLMDEYNLHKRVKKDVEFLSKELESMHAALIKVGDVPRDQLDRQVKLWAGEVRELSYDMEDVVDKFLVRVDGIHPHDDANRFKGLMKRMIGVFKKGKNHHRIADAIKDIKEELKEVAARRDRNKVDGSAPNPIKAIPIDPRLRALYIEATELVGIYGKRDQDLLRLLSIEGDDACNKRLKKISIVGFGGLGKTTLARAVYEKIKGVFDCRAFVPVGQSPDIKKVFKDILIDLGKSHSDVAMLDNRQLINKLHEFLENKRYLIIIDDIWDQNLWKDINIAFSIMNNLGSRLITTTRILNVSESCCSSSNDSIYQMEPLSTDDSRRLFCKRIFPSDTGCPNEFEQVSRDILKKCGGVPLAIITIASALAGGEKVKPKHEWDILLQSLGSGLTEDNSLDEMRRILSFSYYDLPSHLRTCLLYLSIYPEDREIDRDSLIWKWVAEGFVHHGNQGTSLFLLGLDYFNQLINRSMIQPIYDTIGEVYACRVHDMVLDLICNLSYKAKFVNLLDGTGNSMSSQSNCRRLSLQKRNEDHQARPLTDIKSMSRMRSITIFPPAIKLMPSLSRFEVLRVLDLLGCNLGKNTNLQLNLKDVGHLIHLRYLGLEDTKISKLPAEIGKLQFLKVLDLGRNYNLNELSSTVCNFRRLIYLNLVGCQVVPPVGVLQNLRATEVLRGILVSLNIIAQELGNLKRLRELEICFTDGSLDLYEGFVKSLCNLHHIESLCIHDYSEETSSFELMVLLGERWVPPVHFREFLSSMPSQLSALRGWIKRDPSHLSNLFELILWQVKEVQQEDVEIIGRLRSLRCLWIKESTHQTQRLLVIRADGFRCMVNFELDCGSATQILFEPGALLRAEAVSFSLGVRVAKEDGNCGFDLGLQGNLLSLRQPAIHCGGVRVGEAKEAEAAVRHALDAHPNHPAIAISMFPYIAEGAQDDD

SEQ ID NO: 61 - protein, 943 aa, Hordeum vulgare
MEVVTGAMGSLLPKLGQLLMDEYNLHKRVKKDVRFLSRELESMHAALVKVGDVPRDQLDTQVKLWADEVRELSYDMEDVVDKFLVRVDGIHPHDDANRFKGLMKRMVGVFKKGKNHHRIADAIKDIKEQLQEVAARRDRNKVDGSAPNPIKAIPIDPRLRALYIEATELVGIYGKRDQDLLRLLSIEGDDACNKRLKKISIVGFGGLGKTTLVRAVYEKIKGVFDCRVFVPVGQNPDIKKIFKDILIDLGKSHSDVAMLDERQLINKLHEFLENKRYLIIIDDIWDQNLWKDINIAFSNRNNLGSRLITTTRILNLSESCCSSSDDSIYQMEPLSTDDSRRLFCKRIFPSETVCPNEFEQVSRDILKKCGEVPLAIITIASALAGGEKVKPKHEWDILLQSLGSGLTEDNSLDELRRILSFSYYNLPFHLRTCLLYLSIYPEDSMIDRDSLIWKWVAEGFVHHGNQGTSLFLLGLNFFNQLINRSLIQPIYSFSGDVHACRVHDMVLDLICNLSHEAKFVNLLDGTGNSMFSQSNCRRLSLQNRNEDHQAKPLTDIKSMSRVRSITIFPPAIEVMPSLSRFDVLRVLDLSKCILGENSSLQLNLEDVGHLIHLRYLGLEGTKISKLPAEIGKLQFLEVLDLEDNHNLNELPFTVCNFRRLTYLNLVGCQVFPLVGVLQNLTSIEVLRGIWVSLKIIAQELGNLERLRELEIYEFVNSLCNLHHIESLRIECNSRETSSFELMDLLGERWLPPVHLRRFVSSMPSQLSALRGWIKRDPSHLSNLSELILSSVKDVQQDDVEIIGGLLCLRRLEIITSTDQTQRLLVIRADGFRCTVDFRLDCGSATQILFEPGALPRAVRVWFSLGVRVTKEDGNRGFDLGLQGNLFSLREFVSVYMYCGGARVGEAKEAEAAVRRALEAHPSHPRIYIQMRPHIAKGTHPAPN

SEQ ID NO: 62 - protein, 967 aa, Hordeum vulgare
MHVVTGAMGSLLPKLGQLLMEEYKLHKRVKNDVEFLRKELESMHTALIKVGEVPRHQLDKQVKLWADEVRDLSYNMEDVVDKFLVRVDGVDPHDNTDRFKGIMRKMIGLFKKGKNHHQIADAIKEIKEQLQEVAARRDRNKVEGIASNPMEAIPIDPCLRALYAEATELVGIYGKRDEDIMRLLSMEGEDDASNKRLKKVSIVGFGGLGKTTLAKAVYENIKGDFDCRAFVPVGQNPDMKKVFRDILIDLRVSNSELAELDERQLINKLHEFLENKRYLVIIDDIWDAKLWERINFAFSNRNNLGSRLIITTRIFSVSKSSCMLPDDAVYEMKPLSDDDSRSLFYKRIFPSESGCPNEFEQVSEDILKKCGGVPLAIITIASSLASGQKVKPKSEWDILLQSLGSGLTKDNSLEEMRRILSFSYYDLPDHLKTCLLYLCIYPEDSMIDRDRLIWKWVAEGFIHQGNQGTSLFLLGLNYFNQLINRSMIQPIYDGLGEVSACRVHDMVLDLICNLSHEAKFVNVLNGTRDSMSSQSNVRRLSLQDGSKDHQGRPLRNFTGISRVRSITIFPPAINIMPALSRLEVLRVLDLYHCNLGKNSSLQHRLRDVGHLIHLRYLGLAGTKISELPAEIGNLQFLEVLDLEDNSELRNLSSTICKLRRLICLHVHRDEVAPGVLQNLTSIEVLRRLVVSLNTVAQELGNLVRLRELLVCFINVGLDLYEGFVKSVCNLHHIESLRIYSVRASSELMDLLGERWVPPGHLRRFEAHMPSQLSALRGWIMRDPLHLSNLSDLVLTSVKEVQQEDMEIIGGLLSLRGLQIKSTQQTQRLLVIRADVFRCMICFDLDCGSGAQIVFEPGALPRTEGLRFSLGVRVTKEDGNHGFDLGLQGNLLSLRTFVWVQIYCGGARVGEAKEADAVVRHTLRSHPNHPGIISLMFNMIPNIAEGTHQSPRTLLINYSCTYVCAFSQ

SEQ ID NO: 63 - protein, 939 aa, Hordeum vulgare
MELVAGAMVSLISKLGKLLTEEYNLRKSVKKNVEFLRRELEIMHTVLIKVDEVPREQLDSQVKIWADEVRELSYNMEDVIDKCLVRVDNIQSHDNANGLERLMKRMIVVFKKGKNHHRIANAITEITEQFHELAARPERNKVDGITPNPTAIVLDPRLRALYTEVTELVGISGKRDEDIMRLLSMETEDDASNKRLKKVSIVGFGGLGKTTLAKAVYEKIKGDFDCRAFVPIGRNPDIKKVFRDILIELGNSHSDLTILDAKQLMVKLREFLENKRYLVIIDDIWDESLWEIIKFAFSNRNNLGSRLITTTRIVSVSNSCCSSADDSVYQMKPLSLDDSRKLFHKRIFSSETECPNEFEQVSRDILKKCGGVPLAIITIASALAGGQKVKPKHEWYILLQSLGSGLTEDNSLDEMRRILSFSYYDLPYDLRTCLLYLSIYPEGSEIGRDRLIWKWVAEGFVHPGNQGTSLFLLGLNYFNQLINRSMIQPIYDHLGQISTCRIHDMVLDLICNLSHEAKFVNLLDGTRNSMSSQSNVRRLSLQNINEDHPAKSLTNIMSMSRVRSITIFPTAIDIMPALSRFKALRVLDLMGCNLGENSNLQLHLKDVGHLIHLRYLGLSRTKIRELPPKIGNLQFLEVLDLGNNYIDELPPTVCNLRRLIYLNIYPCKVVPTGVLHNLTSMEVLREILVPLNIIAQELGNLARLRELRIHFKDGHYDLYEGFVKSLCNLHRMESLSIDCNYGETSFELMDLLAERWVPPVHLREFVSRMPSKLSALRGWIKKDPSHLSNLSVLFLWPVKEVQQEDVEIIGGLQSLRRLWMKSTQQIQRLLIIRADVFLCMVDFGLYCGSAAQIMFEPGALPRAEYVRFSLGVRVAKEDGNYGFDLGLQGNLLSLRQRVWVNLYCGGAFYPKPSLKVQKLNRLIATCKEAKIPNFSFLCK

SEQ ID NO: 64 - DNA, 3989 bp, Hordeum vulgare
atggatattgtcaccggtgccatttccaacctgattcccaagttgggggagctgctcacggaggagttcaagctgcacaagggtgtcaagaaaaatattgaggacctcgggaaggagcttgagagcatgaacgctgccctcatcaagattggtgaggtgccgagggagcagctcgacagccaagacaagctctgggccgatgaagtcagagagctctcctacgtcattgaggatgtcgtcgacaagttcctcgtacaggttgatggcattcagtttgatgataacaacaacaaatttaaggggttcatgaagaggacgaccgagttgttgaagaaagtcaagcataagcatgggatagctcacgcgatcaaggacatccaagagcaactccaaaaggtggctgataggcgtgacaggaacaaggtatttgttcctcatcctacgagaacaattgctattgacccttgccttcgagctttgtatgctgaagcgacagagctagttggcatatatggaaagagggatcaagacctcatgaggttgctttccatggagggcgatgatgcctctaataagagactgaagaaggtctccattgttggatttggagggttgggcaagaccactcttgctagagcggtatacgagaagattaaaggtgatttcgattgtcgggcttttgttccggtcggtcagaaccctcacatgaagaaggttttaagggatatcctcattgatctcggaaatcctcactcagatcttgcgatgctggatgccaatcagcttattaaaaaacttcgtgaatttctagagaacaaaaggtatgcatcaatttagaaaaaagtacactattatgtgatgtttgtttcctatgctagtggaacggattagaatttttttttcattaaggtcacctttactggcataagcagttcacactaaacggtaaaccttataggtgaaaattttcaggcatatatatatatatatatatatatatatgtttgattctttccggcttaacaaaataattagcaagtacttcttgttgcatttgttccaacggctgaatttattggcatcggtccaagaaatccatctaaatgttttacatttcaccaaagtgtgtgtcatgacagatgtaacaaataataaaccaaaaggagaggaaggaaagaggaagataaatgttacaaaaatttaaatcaaacttatttctacctttctccttacctacccagtttaaaaacacatattatattttaaagagaggcaacatgcgccaaaggctacccttgaaaattcctaaaatattgtacatttgactgatgaccaaacaaaaagttaaattgtctcttccttatcacattatatttccatgcatgcctttttctggaaacttactatcagcaaaatttagatgaaaggataatgccacataatttcagtctccaagagatttgttagttgtcatatattaaattggtgggccaatctattcctgggtctttttatgtatctacttgaccatttgaacttctgtagttaattgtattctatgaatgatcactcatccaaaaacttgttatttgtgttttactctgttgaatcttgaatatttattcattttgttcatcatacgattggaggcccataatagatgcttaatgagagtaagattatcgatctccaaacacatgcttcttactagtgttgaatatatacccttttagatgtatagttcaacccatagattcatatgaccctcagctttctgatgtgtatgtatgaccttacactgacactctgaactaatgtaggtatcttgtcataattgatgatatatgggatgaaaaattatgggaaggcatcaactttgctttctccaataggaataatctaggcagtcggctaatcaccacaacccgcattgtcagtgtctctaattcatgttgctcatcacatggtgattcggtttatcaaatggaaccactttctgttgatgactccagaatactcttctggaaaagaatatttccagatgagaatggatgtctaaatgaatttgaacaagtgtcgagagatattctaaagaaatgtggtggggtaccactagccataattaccatagctagtgctttggccggtgaccagaagatgaaaccaaagtgtgagtgggatattctccttcagtcccttggctctggactaacagaagataacagtttagaggagatgcggagaatactctctttcagctattctaatctaccttctcatctgaaaacttgtctactgtatctatgtatatatccagaagatagcaagattcatagagatgaactgatatggaagtgggtggccgaaggatttgtccaccatgaaaaccaaggaaatagcttgtatttgctcggattaaattacttcaaccagctcattaatagaagtatgatccagcccatatatggttttaatgacgaggtatatgtatgtcgtgtacatgatatggttctggaccttatctgcaacttgtcacgtgaagcaaaatttgtgaatctattggatggcagtgggaatagcatgtcttcacagggtaattgtcgccgtctgtcccttcaaaaaagaaatgaagatcatcaagccaaacctatcacagatatcaagagtatgtcacgagtgaggtcaattactatctttccacctgctattgaagtcatgccatctctttcaaggtttgacgttttacgtgtacttgatctgtcacgatgtaatcttggggagaatagcagcctgcagcttaacctgaaggatgttggacatttaactcacctaaggtaccttggtctagaaggtaccaacatcagtaagctccctgctgagataggaaaactgcagtttttggaggtgttggatcttggaaacaatcataatctaaaggaattgccgtccactgtttgtaatttcagaagattaatctacctaaatttatttgggtgtccggtggttcctccagttggtgtgttgcaaaatctgacatccatagaagtgttgagggggatcttggtctctgtgaacattattgcacaagagcttggcaacctggaaaggctgagggtgcttgatatttgcttcagggatggtagtttggatttgtataaagatttcgtgaagtctctgtgcaacctacatcacatcgaaagtctacgtattgagtgcaattccagagaaacatcatcttttgaactggtggatctcttgggagaacgctgggtgcctcctgtacatttccgtgaatttgtgtcatccatgcctagccaactctctgcactgcgagggtggataaagagagacccctcccatctctcgaacctctccgagttaatcctctcgtcagtgaaggacgtgcagcaggatgacgtggaaatcattggggggttgttgtgccttcgtcgtctctttataataacgagcaccgaccaaacgcaacggctgctagtcatccgtgcagatgggttccgctgtacggttgactttcgattggattgtggatctgccacgcagatattgtttgaaccaggagctttgccaagggcggtaagagtttggttcagccttggcgtgcgggtgacgaaagaggatggtaaccgtggcttcgacttgggcctgcaggggaacctgttctcccttcgagagtttgtctctgtttatatgtattgtggtggagcgagggttggggaggcaaaggaagcggaggctgcggtgaggcgtgccctggaagctcatcccagccatccccggatttatattcagatgaggccgcatatagcaaaaggtacgcatcctgcacctaactaattacttgtgcacttacacatgtgtttttttctcaatgacggactgaccttattactttctgcatggattttgatctctaaatctcccaaggtgctcatgatgacgatttgtgtgaggacgaggaggagaactga

SEQ ID NO: 65 - DNA, 3976 bp, Hordeum vulgare
atggatattgtcaccggtgccatttccaacctgattcccaagttgggggagctgctcacggaggagttcaagctgcacaagggtgtcaagaaaaatattgaggacctcgggaaggagcttgagagcatgaacgctgccctcatcaagattggtgaggtgccgagggagcagctcgacagccaagacaagctctgggccgatgaggtcagagagctctcctacgtcattgaggatgtcgtcgacaaattcctcgtacaggttgatggcattcagtctgatgataacaacaacaaatttaaggggctcatgaagaggacgaccgagttgttgaagaaagtcaagcataagcatgggatagctcacgcgatcaaggacatccaagagcaactccaaaaggtggctgataggcgtgacaggaacaaggtatttgttcctcatcctacgagaccaattgctattgacccttgccttcgagctttgtatgctgaagcgacagagctagttggcatatatggaaagagggatcaagacctcatgaggttgctttccatggagggcgatgatgcctctaataagagactgaagaaggtctccattgttggatttggagggttgggcaagaccactcttgctagagcggtatacgagaagattaaaggtgattttgattgtcgggcatttgttccggtcggtcagaaccctgacatgaagaaggttttaagggatatcctcattgatctcggaaatcctcactcagatcttgcgatgctggatgccaatcagcttattaaaaagcttcatgaatttctagagaacaaaaggtatgcatcaatttagaaaaaagtacactattatgtgatgtttgtttcctatgctagtggaacggattagaatatttttttcatcaaggtcacctttactggcataagcagttcacactaaacagtaaaccttataggtgaaaaatttcaggcatgtatatatatatatatatgtttgattctttccggcttaacaaaataattagcaagtacttcttgttgcatttgttccaacggctgaatttattggcaccagtccaagaaatccatctaaatgttttacatttcaccaaagtgtgtgtcatgacagatgtaacaaataataaaccaaaaggagaggaaggaaagaggaagataaatgttacaaaaatttaaatcaaacttatttctacctttctccttacctacccagttgtaaaacacatattatattttaaagagaggcaacatgcgccaaaggctgcccttgaaaattcctaaaatattgtacatttgactcatgaccaaacaaaaagttaaattgtctcttccttatcgcattatatttccatgcatgcctttttctggaaacttactattagcaaaatttagacgaaaggatgatgccacataatttcagtctccagagatttgttagttgccatatattaaattggkgtgccaatctatacctgggccttttttatgtatctacttgatcatttgaacttctgtagttaattgtattctatgaatgatcactcatccaaaaacttgctatttgtgtttcactttgttgagtcttgaatatttattcattttgttcatcatacgattggaggcccataatggatgcttaatgagagtaagattatcgagctccaaacacatgcttcttactagtgtttgaatatatagccttatagatgtatagttcaacccatagattcatatgaccctcagctttctgatgtgtatatataaccttacactgacactgtgaattaatgtaggtatcttgtcataattgatgatatatgggatgaaaaattgtgggaaggcatcaactttgctttctccaataggaataatctaggcagtcgactaatcaccacaacccgcattgtcagtgtctctaattcatgttgctcatcagatggtgattcagtttatcaaatggaaccgctttctgttgatgactctagaatgctcttctccaaaagaatatttcctgatgagaatggatgtataaatgaatttgaacaagtatccagagatattctaaagaaatgtggtggggtaccactagccataattactatagctagtgctttggctggtgaccagaagatgaaaccaaaatgtgagtgggatattctccttcggtcccttggctctggactaacagaagataacagtttagaggagatgcggagaatactctctttcagctattctaatctaccttcgcatctgaaaacttgtctactgtatctatgtgtatatccagaagatagtatgatttctagagataaactgatatggaagtgggtggctgaaggatttgtccaccatgaaaatcaaggaaatagcctgtatttgctcggattaaattacttcaaccagctcattaatagaagtatgatccagccaatatataattatagcggcgaggcatatgcttgccgtgtacatgatatggttctggaccttatctgcaacttgtcatatgaagcaaagtttgtgaatctattggatggcactgggaatagcatgtcttcacagagtaattgtcgccgtttgtcccttcaaaaaagaaatgaagatcatcaagtcaggcctttcacagatatcaagagtatgtcacgagtgaggtcaattactatctttccatctgctattgaagtcatgccatctctttcaaggtttgacgttttacgtgtacttgatctgtcacgatgtaatcttggggagaatagcagcctgcagcttaacctcaaggatgttggacatttaactcacctaaggtaccttggtctagaaggtaccaacatcagtaagctccctgctgagataggaaaactgcagtttttggaggtgttggatcttggaaacaatcgtaatataaaggaattgccgtccacagtttgtaatttcagaagattaatctacctaaatttagttggctgtcaggtggttcctccagttggtttgttgcaaaatctaacagccatagaagtgttgaggggtatcttggtctctctgaacattattgcacaagagcttggcaagttgaaaagtatgagggagcttgagattcgcttcaatgatggtagtttggatttgtatgaaggtttcgtgaagtctctttgcaacttacatcacatagaaagcctaatcattggttgcaattctagagaaacatcatcttttgaagtgatggatctcttgggagaacggtgggtgcctcctgtacatctccgtgaatttgagtcgtccatgcctagccaactctctgcactgcgagggtggataaagagagacccctcccatctctcaaacctctccgacttagtcctgccagtgaaggaagtgcaacaggatgacgtggaaatcattggggggttgctggcccttcgccgtctctggataaagagcaaccaccaaacacaacggctgctagtcatccctgtagatgggttccactgtattgttgactttcagttggactgtggatctgccacgcagatattgtttgagcctggagctttgccgagggcagaatcagttgtgatcagtctgggcgtgcgggtggcgaaagaggatggtaaccgtggcttcgacttgggcctgcaagggaacttgctatcccttcggcggcatgtctttgttcttatctattgtggtggagcgagggttggggaggcaaaggaagcgaaggctgcgctgaggcgtgcccaggaagctcatcccgaccatctccggatttatattgacatgaggccgtgtatagcagaaggtatcgcatgttgcacctaactaattacttgtgcacttacgcatgtgttttttttctcaatgaccgactaaccttattactttctgtgttggttttgatctctaaatctcccaaggctcatgatgacgatttgtgtgagggcgaggaggagaactaa

SEQ ID NO: 66 - DNA, 4049 bp, Hordeum vulgare
atggatattgtcaccggtgccatttccaacctgattcccaagttgggggagctactcacggaggagttcaagctgcacaagggtgtcaagaaaaatattgaggacctcgggaaggagcttgagagcatgaacgctgccctcatcaagattggtgaggtgccgagggagcagctcgacagccaagacaagctctgggccgatgaggtcagagagctctcctacgtcattgaggatgtcgtcgacaagttcctcgtacaggttgatggcattaagtctgatgataacaacaacaaatttaaggggctcatgaagaggactaccgagttgttgaagaaagtcaagcataagcatgggatagctcacgcgatcaaggacatccaagagcaactccaaaaggtggctgataggcgtgacaggaacaaggtatttgttcctcatcctacgagaacaattgctattgacccttgccttcgagctttgtatgctgaagcgacagagctagttggcatatatggaaagagggatcaaggcctcatgaggttgctttccatggagggcgatgatgcctctaataagagactgaagaaggtctccattgttggatttggagggttgggcaagaccactcttgctagagcggtatacgagaagattaaaggtgatttcgattgtcgggcatttgttccggtcggtcagaaccctgacatgaagaaggttttaagggatatcctcattgatctcggaaatcctcactcagatcttgcgatgctggatgccaatcagcttattaaaaagcttcatgcatttctagagaacaaaaggtatgcatcaatttagaaaaaagtacactattatgtgatgtttgtttcctatgctagtggaacggattagaatatttgttgtcattaacgtcacctttactggcataagcagttcacactaaacagtaaaccttataggtgaaaattttcaggcatatatatatatatatatatatatatatatatatatatatatatatatatatatatatatatatatatatatgtttgattctttccggcttaacaaaataattagcaagtacttcttgttgcatttgttccaacggctgaatttattggcatcagtccaagaaatccatctaaacgttttacatttcaccaaagtgtgtgtcatgacagatgtaacaaataataaaccaaaaggagaggaaggaaagagggagataaatgttacaaaaatttaaatcaaacttatttctacctttctccttacctacccagttgtaaaacacatattatattttaaagagaggcaacatgcgccaaaggctgcccttgaaaattcctaaaatattgtacatttgactcatgaccaaacaaaaagttaaattgtctcttccttatcgcattatatttccatgcatgcctttttctggaaacttactatcagcaaaatttagacgaaaggatgatgccacataatttcagtctccaagagatttgttagttgccatatattaaattggtgtgccaacctatacctgggccttttttatgtatctacttgatcatttgaacttctgtagttaattgtattctatgaatgatcactcatccaaaaacttgctatttgtgtttcactttcttgagtcttgaatatttattcattttgttcatcatacgattggaggcccataatggatgcttaatgagagtaagattatcgagctccaaacacatgcttcttactagtgtttgaatatatagccttatagatgtatagttcaacccatagattcatatgaccctcagctttctgatgtgtatatataaccttacactgacactccgaaataatgtaggtatcttgtcataattgatgatatatgggatgaaaaattgtgggaaggcatcaactttgctttctccaataggaataatctaggcagtcggctaatcaccacaacccgcattgtcagtgtctctaattcatgttgctcatcagatggtgattcagtttatcaaatggaaccgctttctgttgatgactccagaatgctcttctacaaaagaatatttcctgatgagaatgcatgtataaatgaatttgaacaagtatccagagatattctaaagaaatgtggtggggtaccactagccataattactatagctagtgctttggctggtgaccagaagatgaaaccaaaatgtgagtgggatattctccttcggtcccttggctctggactaacagaagataacagtttagaggagatgcggagaatactctctttcagctattctaatctaccttcgaatctgaaaacttgtctactgtatctatgtgtatatccagaagatagtatgatttctagagataaactgatatggaagtgggtggccgaaggatttgtccaccatgaaaatcaaggaaatagcctgtatttgctcggattaaattacttcaaccagctcattaatagaagtatgatccagccaatatataattatagcggcgaggcatatgcttgccgtgtacatgatatggttctggaccttatctgcaacttgtcacgtgaagcaaagtttgtgaatctattggatggcactgggaatagcatgtcttcacagagtaattgtcgtcgtttgtcccttcagaaaagaaatgaagatcatcaagccaggcctctcatagatatcaagagtatgtcacgagtgaggtcaattactatctttccacctgctattgaagtcatgccatctctttcaaggtttgaggttttatgtgtacttgatttgtcgaaatgtaatcttggggaggatagcagcctgcaacttaacctcaaggatgttggacaattaattcagctaaggtaccttggtctagaatgtaccaatataagtaagctcccgactgagataggaaaactgcagtttttggaggtgttggatcttggaaacaatcctaatctaaaagaattgccgtccactattcgtaatttcagaagattaatctacctaaatttagttggctgtcaggtgattcctccagtgggtgtgttgcaaaatctgacatccatagaagtattgaggggtatcttggtctatctgaacattattgcacaagagcttggcaacctggaaagggtgagagatcttgagattcgcttcaatgatggtagtttggatttgtatgaaggtttggtgaattctctgtgcaacctacatcacatcgaaagtctaaatattcgttgcaatcccggagaaacatcatcttttgaactgatggatctcttggaagaacgttgggtgccgcctgtacatctccgtgaatttaagtcattcatgcccagccaactctctgcactgcgagggtggatacagagagacccctcccatctctcgaacctctccgagttaaccctctggccagtgaaggacgtgcagcaggatgacgtggaaatcattggggggttgttgtcccttcgtcgtctctggatagtaaagagcatccaccaaacgcaacggctgctagtcatccgtgcagatgggttccgctctatggttgaatttcgtttggattgtggatctgccacgcagatattgtttgagccaggagctttgccgagggcggaatcagttgtgatcagtctgggcgtgcgggtggcgaaagaggatggtaaccgtggcttcgacttgggcctgcaggaagcaaaggatgtctcccttcggtgggatgtctttgttcttctctattgtggtggagcgagggttggggaggcaaaggaagcggaggctgcggtgaggcgtgccctggaagctcatcccagacatcctcggatttatattgacatgaggccggatatacaggaaggtacacacactgcatctaactaactacttctgcatttacgtctctgctttttcaatgttctcaatgacggaccgatcttattacttcctgcatggatttgatttctgaactcccaaggtgctcatgatgaccatttgtgtgagaacgaggacgagggtgagaactga

SEQ ID NO: 67 - DNA, 4052 bp, Artificial Sequence
  Consensus
atggatattgtcaccggtgccatttccaacctgattcccaagttgggggagctgctcacggaggagttcaagctgcacaagggtgtcaagaaaaatattgaggacctcgggaaggagcttgagagcatgaacgctgccctcatcaagattggtgaggtgccgagggagcagctcgacagccaagacaagctctgggccgatgaggtcagagagctctcctacgtcattgaggatgtcgtcgacaagttcctcgtacaggttgatggcattcagtctgatgataacaacaacaaatttaaggggctcatgaagaggacgaccgagttgttgaagaaagtcaagcataagcatgggatagctcacgcgatcaaggacatccaagagcaactccaaaaggtggctgataggcgtgacaggaacaaggtatttgttcctcatcctacgagaacaattgctattgacccttgccttcgagctttgtatgctgaagcgacagagctagttggcatatatggaaagagggatcaagacctcatgaggttgctttccatggagggcgatgatgcctctaataagagactgaagaaggtctccattgttggatttggagggttgggcaagaccactcttgctagagcggtatacgagaagattaaaggtgatttcgattgtcgggcatttgttccggtcggtcagaaccctgacatgaagaaggttttaagggatatcctcattgatctcggaaatcctcactcagatcttgcgatgctggatgccaatcagcttattaaaaagcttcatgaatttctagagaacaaaaggtatgcatcaatttagaaaaaagtacactattatgtgatgtttgtttcctatgctagtggaacggattagaatattttttntcattaaggtcacctttactggcataagcagttcacactaaacagtaaaccttataggtgaaaattttcaggcatatatatatatatatatatatatatatnnnnnnnnnnnnnnnnnnnnnnnnnnnnnnnnnnnnnnnnnnnngtttgattctttccggcttaacaaaataattagcaagtacttcttgttgcatttgttccaacggctgaatttattggcatcagtccaagaaatccatctaaatgttttacatttcaccaaagtgtgtgtcatgacagatgtaacaaataataaaccaaaaggagaggaaggaaagaggaagataaatgttacaaaaatttaaatcaaacttatttctacctttctccttacctacccagttgtaaaacacatattatattttaaagagaggcaacatgcgccaaaggctgcccttgaaaattcctaaaatattgtacatttgactcatgaccaaacaaaaagttaaattgtctcttccttatcgcattatatttccatgcatgcctttttctggaaacttactatcagcaaaatttagacgaaaggatgatgccacataatttcagtctccaagagatttgttagttgccatatattaaattggtgtgccaatctatacctgggccttttttatgtatctacttgatcatttgaacttctgtagttaattgtattctatgaatgatcactcatccaaaaacttgctatttgtgtttcactttgttgagtcttgaatatttattcattttgttcatcatacgattggaggcccataatggatgcttaatgagagtaagattatcgagctccaaacacatgcttcttactagtgtttgaatatatagccttatagatgtatagttcaacccatagattcatatgaccctcagctttctgatgtgtatatataaccttacactgacactctgaantaatgtaggtatcttgtcataattgatgatatatgggatgaaaaattgtgggaaggcatcaactttgctttctccaataggaataatctaggcagtcggctaatcaccacaacccgcattgtcagtgtctctaattcatgttgctcatcagatggtgattcagtttatcaaatggaaccgctttctgttgatgactccagaatgctcttctncaaaagaatatttcctgatgagaatggatgtataaatgaatttgaacaagtatccagagatattctaaagaaatgtggtggggtaccactagccataattactatagctagtgctttggctggtgaccagaagatgaaaccaaaatgtgagtgggatattctccttcggtcccttggctctggactaacagaagataacagtttagaggagatgcggagaatactctctttcagctattctaatctaccttcgcatctgaaaacttgtctactgtatctatgtgtatatccagaagatagtatgatttctagagataaactgatatggaagtgggtggccgaaggatttgtccaccatgaaaatcaaggaaatagcctgtatttgctcggattaaattacttcaaccagctcattaatagaagtatgatccagccaatatataattatagcggcgaggcatatgcttgccgtgtacatgatatggttctggaccttatctgcaacttgtcacgtgaagcaaagtttgtgaatctattggatggcactgggaatagcatgtcttcacagagtaattgtcgccgtttgtcccttcaaaaaagaaatgaagatcatcaagccaggcctntcacagatatcaagagtatgtcacgagtgaggtcaattactatctttccacctgctattgaagtcatgccatctctttcaaggtttgacgttttacgtgtacttgatctgtcacgatgtaatcttggggagaatagcagcctgcagcttaacctcaaggatgttggacatttaactcacctaaggtaccttggtctagaaggtaccaacatcagtaagctccctgctgagataggaaaactgcagtttttggaggtgttggatcttggaaacaatcntaatctaaaggaattgccgtccactgtttgtaatttcagaagattaatctacctaaatttagttggctgtcaggtggttcctccagttggtgtgttgcaaaatctgacatccatagaagtgttgaggggtatcttggtctctctgaacattattgcacaagagcttggcaacctggaaaggntgagggagcttgagattcgcttcaatgatggtagtttggatttgtatgaaggtttcgtgaagtctctgtgcaacctacatcacatcgaaagtctaantattggttgcaattccagagaaacatcatcttttgaactgatggatctcttgggagaacgntgggtgcctcctgtacatctccgtgaatttgagtcatccatgcctagccaactctctgcactgcgagggtggataaagagagacccctcccatctctcgaacctctccgagttaatcctctngccagtgaaggacgtgcagcaggatgacgtggaaatcattggggggttgttgtcccttcgtcgtctctggatantaaagagcanccaccaaacgcaacggctgctagtcatccgtgcagatgggttccgctgtatggttgactttcgnttggattgtggatctgccacgcagatattgtttgagccaggagctttgccgagggcggaatcagttgtgatcagtctgggcgtgcgggtggcgaaagaggatggtaaccgtggcttcgacttgggcctgcaggggaacntgnnnntctcccttcggnggnatgtctttgttcttatctattgtggtggagcgagggttggggaggcaaaggaagcggaggctgcggtgaggcgtgccctggaagctcatcccagccatccccggatttatattgacatgaggccgnatatagcagaaggtancgcatnctgcacctaactaattacttgtgcacttacgcatgtgttttttnnnnnttctcaatgacggactgaccttattactttctgcatnggttttgatctctaaatctcccaaggtgctcatgatgacgatttgtgtgagnnnnnngacgaggaggagaactga

SEQ ID NO: 68 - protein, 18 aa, Artificial Sequence
  Consensus motif
LXXLXXLXXLXLXXXXXL